(19) United States
(12) Patent Application Publication
(10) Pub. No.: US 2026/0176659 A1
(43) Pub. Date: Jun. 25, 2026

(54) IMPROVED METHOD FOR THE PRODUCTION OF NATURAL VANILLIN

(71) Applicant: BASE SE, Ludwigshafen am Rhein (DE)

(72) Inventors: Elisa LAFRANCHI, Graz (AT); Wolfgang KROUTIL, Graz (AT); Christian WILLRODT, Ludwigshafen am Rhein (DE); Valerio FERRARIO, Ludwigshafen am Rhein (DE)

(21) Appl. No.: 19/126,281

(22) PCT Filed: Oct. 31, 2023
(86) PCT No.: PCT/EP2023/080329

§ 371 Date: May 1, 2025

(30) Foreign Application Priority Data
Nov. 2, 2022 (EP) 22205082.5

(51) Int. Cl. C12P 7/24; C12N 9/04; C12N 9/42; C12N 9/88; C12N 15/81; C12P 7/42

(52) U.S. Cl. CPC C12P 7/24; C12N 9/0006; C12N 9/2437; C12N 9/88; C12N 15/81; C12P 7/42

(57) ABSTRACT

The present invention is in the field of biocatalysis, bio-conversion and fermentation and is directed to a cascadic method for the enzymatic production of vanillin from eugenol.

### FIELD OF THE INVENTION

The present invention is in the field of biocatalysis, bio-conversion and fermentation and is directed to a cascadic method for the enzymatic production of vanillin from eugenol.

### INTRODUCTION

Vanillin (4-Hydroxy-3-methoxybenzaldehyde, FEMA 3107) is a worldwide known flavouring agent, which is used in a plethora of products i.e. food, beverages as well as cosmetics, pharmaceuticals and many others. Traditionally, natural vanillin is obtained from the beans or pods of vanilla, a tropical climbing vine from the orchid family. The cost of this highly demanded flavour—second after saffron—historically encouraged the development of chemical methods for the production of its synthetic equivalent. Less than 20 years after vanillin isolation and the elucidation of its chemical structure (Gobley 1858), its first synthetic route from eugenol was established and industrialised in France and United States until 1920s (M. B. Hocking, J. Chem. Educ. 1997, 74, N. J. Gallage, B. L. Møller, Mol. Plant 2015, 8). Nowadays synthetic vanillin is mostly produced from lignin or petrochemicals (guaiacol) (N. J. Gallage, B. L. Møller, Mol. Plant 2015, 8, 40-57). However, since the late 1970s, authorities and consumers have raised concerns for the extensively use of the artificial flavour versus its natural equivalent, such as the environmental impact of the processes, food safety and customer desires.

Natural vanillin may be also obtained by taking a precursor from natural sources and transform it to vanillin by biotechnological means, including enzymatic synthesis (N. J. Gallage, B. L. Møller, Mol. Plant 2015, 8, M. García-Bofill, et al., Appl. Catal. A Gen. 2019, 582). To date, the fermentation of a number of microorganisms has been extensively explored. One of the most studied approach is the biotransformation of ferulic acid, which can be retrieved from agrowaste since it is part of the plant cell walls P. Barghini, et al, Microb. Cell Fact. 2007, 6, 1-11, N. Graf, J. Altenbuchner, Appl. Microbiol. Biotechnol. 2014, 98, 137-149. A. I. Galadima, et al, Biomass Convers. Biorefinery 2020, 10, 589-609). Additional efforts focus on the development of eugenol degrading strains, thus this starting material is cheap and it can be easily obtained from clove oil in large quantities. For instance, wild type microorganisms such as Pseudomonas sp. HR199 are capable to oxidise eugenol to ferulic acid in 3 enzymatic steps, which is then converted to vanillin by ferulic acid CoA synthase and enoyl-CoA hydratase aldolase (Priefert et al. Arch. Microbiol. 1999, 172, 354-363). Unfortunately, the desired flavour is toxic for the cells and it is further metabolised via oxidation (vanillic acid) and demethylation (protocatechuic acid; Galadima, et al, Biomass Convers. Biorefinery 2020, 10, 589-609). To overcome these problems strains were engineered by—for example—disrupting the vanillin dehydrogenase gene, achieving a molar yield of 44.6% (equal to 2.9 mM of vanillin).

Alternatively, part of the Pseudomonas pathway described above was introduced in E. coli together with the vanillyl alcohol oxidase (VAO) from Penicillium simplicissimum, and 0.3 g/L of vanillin and 0.1 g/L of vanillyl alcohol were obtained from a procedure involving two microbial strains, whereas longer incubation time led to a complete loss of the vanillin (Overhage, et al, Appl. Environ. Microbiol. 2003, 69, 6569-6576). Another method known in the prior art involves transforming ferulic acid to 4-vinylguaiacol via a decarboxylase (Fdc) and then the formation of vanillin by a carotenoid cleavage oxygenase (Cso2) (Furuya, et al. Chembiochem 2014, 15, 2248-2254). With this 2-step cascade, 62% conversion of ferulic acid to vanillin was obtained employing mutated variants of Cso2. Also one step enzymatic transformations have been considered: for example, vanillic acid can be reduced to the desired aldehyde by a carboxylic acid reductase with up to 95% conversion (Horvat, G. Fiume, S. Fritsche, M. Winkler, J. Biotechnol. 2019, 304, 44-51). On the other hand, eugenol oxidase efficiently transforms vanillyl alcohol to vanillin giving 85% isolated yield; on top of this, the oxidative process was successfully run with 330 mM substrate loading (García-Bofill, et al., Appl. Catal. A Gen. 2019, 582; García-Bofill, et al., Appl. Catal. A Gen. 2021, 610, 117934).

In view of the prior art, the problem of the present invention relies in the provision of an efficient method for the production of natural vanillin, turning the process cleaner (milder conditions, enzymes instead of polluting reagents) and giving access to the more valuable natural vanillin as product.

To solve this problem, the inventors of the present invention designed an unprecedented cascade for the enzymatic synthesis of vanillin, simpler than the aforementioned microbial pathways. In this regard, the inventors of the present invention utilized the analogy between the speculated mechanism for positional isomerisation of eugenol to isoeugenol and the reaction catalysed by vanillin alcohol oxidases (VAO-type oxidases), where eugenol is converted to coniferyl alcohol via formation of a p-quinone methide intermediate (Martin, et al., in Flavin-Dependent Enzym. Mech. Struct. Appl. (Eds.: P. Chaiyen, F.B.T.-T.E. Tamanoi), Academic Press, 2020, pp. 63-86; Ewing, et al., in Flavin-Dependent Enzym. Mech. Struct. Appl. (Eds.: P. Chaiyen, F.B.T.-T.E. Tamanoi), Academic Press, 2020, pp. 87-116). Coniferyl alcohol differentiates from isoeugenol only by a hydroxy group at the C-γ atom, thereby its oxidative alkene cleavage leads to vanillin. Whilst VAO-type oxidases are very well described and applied in biocatalysis, an enzymatic 1-step reaction to convert coniferyl alcohol into vanillin has never been disclosed. Thus, the present invention relates to a method for synthesizing natural vanillin from eugenol via a 2-step cascade of novel enzymatic alkene cleaving reactions and their application in a de novo cascade.

### DESCRIPTION OF THE DRAWING

FIG. 1 shows the oxidative cleavage of coniferyl alcohol 2a to vanillin 3a at varied amount of isoeugenol cleavage oxygenase (hereinafter referred to as IECO, alkene cleavage oxygenaseor CO) and catalase after 24 hours. Each reaction (0.5 mL) was set up in 1.5 mL glass vials with the following conditions: Glycine-NaOH buffer (50 mM, pH 9.0), FeSO₄ (1 mM), sodium ascorbate (1 mM), 5% v/v ethanol, IECO (fresh CFE 1-4 mg/mL), bovine liver catalase (Sigma C40; 0-10 mg/mL=0 to ≥100 kU/mL) and coniferyl alcohol (5 mM). Each reaction was incubated at 30° C. and 120 rpm (rotary shaker). To improve oxygenation, the vials were placed horizontally. Error bars indicates the standard deviation from three independent experiments

FIG. 2 shows the extract of the HPLC chromatograms of the enzymatic conversion of eugenol 1a to vanillin 3a, after 24 hours. Black line: 280 nm. Grey line: 340 nm. Reaction conditions: Glycine-NaOH buffer (50 mM, pH 9.0), sodium ascorbate (1 mM), FeSO₄ (1 mM), ethanol 5 vol %, bovine liver catalase (Sigma C40, 10 mg/mL=≥100 kU/mL), eugenol (5 mM), eugenol oxidase (hereinafter referred to as EUGO) (fresh CFE 1 mg/mL), IECO (fresh CFE 4 mg/mL). Each reaction mixture (0.5 mL) was set up in 1.5 mL glass vials and incubated at 30° C. and 120 rpm (rotary shaker). Control 1 was performed in Tris-HCl (50 mM, pH 8.0) and did not contain EUGO. To improve oxygenation, the vials were placed horizontally. Peak were identified by comparison with reference material. The reaction composition at 24 hours is reported in table S3.

FIG. 3 shows the Two-step biocatalytic cascade for the synthesis of vanillin (3a) from eugenol (1a) employing the eugenol oxidase from Rhodococcus jostii (EUGO) and the alkene cleavage oxygenase from Sphingomonadales bacterium (CO-03; SEQ ID NO:30). The side reaction is indicated with a dashed arrow.

FIG. 4 shows SOS-PAGE of E. coli BL21(DE3) cell free extract containing CO-25 (55.4 KDa). Lyophilized cell free extract was reconstituted in buffer to a concentration of 10 mg/mL (buffer salt content was neglected). Then 20, 15 and 10 μg of CFE were loaded on a gel after protein denaturation in Laemmli buffer (Sigma p #53401) at 90° C. M: unstained protein ladder (ThermoFisher 26614).

### LEGEND TO THE SEQUENCE LISTING

| | | Name/Identifier | |
|---|---|---|---|
| Seq-ID | Seq-ID | in Figures or | |
| DNA | PRT | Examples | Organism |
| 1 | 2 | EUGO | Rhodococcus Jostii |
| 3 | 4 | EUGO homolog | Rhodococcus wratislaviensis |
| 5 | 6 | EUGO homolog | Rhodococcus sp. |
| 7 | 8 | EUGO homolog | Rhodococcus opacus |
| 9 | 10 | EUGO homolog | Rhodococcus opacus |
| 11 | 12 | EUGO homolog | Rhodococcus sp. |
| 13 | 14 | EUGO homolog | Rhodococcus koreensis |
| 15 | 16 | EUGO homolog | Gordonia insulae |
| 17 | 18 | EUGO homolog | Rhodococcus sp. |
| 19 | 20 | EUGO homolog | Rhodococcus erythropolis |
| 21 | 22 | EUGO homolog | unknown |
| 23 | 24 | IECO | Pseudomonas nitroreducens |
| 25 | 26 | CO-01 | Thermothelomyces thermophilus |
| 27 | 28 | CO-02 | Pseudonocardia hierapolitana |
| 29 | 30 | CO-03 | Sphingomonadales bacterium |
| 31 | 32 | CO-04 | Rhodococcus sp. |
| 33 | 34 | — | Amycolatopsis alkalitolerans |
| 35 | 36 | CO-05 | Pseudonocardiaceae bacterium |
| 37 | 38 | CO-06 | Novosphingobium sp. |
| 39 | 40 | CO-07 | Thermopolyspora flexuosa |
| 41 | 42 | CO-08 | Amycolatopsis acidiphila |
| 43 | 44 | — | Rhodococcus sp. |
| 45 | 46 | — | Amycolatopsis sacchari |
| 47 | 48 | CO-09 | Streptomyces sp. |
| 49 | 50 | — | Pseudonocardia cypriaca |
| 51 | 52 | CO-10 | Salinibacterium hongtaonis |
| 53 | 54 | — | Streptomyces scabiei |
| 55 | 56 | CO-11 | Trebonia kvetii |
| 57 | 58 | CO-12 | Rhodococcus jostii |
| 59 | 60 | — | Streptomyces sp. |
| 61 | 62 | — | Streptomyces griseorubiginosus |
| 63 | 64 | CO-13 | Actinobacteria bacterium |
| 65 | 66 | CO-14 | Caulobacter segnis |
| 67 | 68 | — | Streptomyces griseorubiginosus |
| 69 | 70 | — | Salinibacterium sp. |
| 71 | 72 | — | Streptomyces sp. |
| 73 | 74 | CO-15 | Herbaspirillum sp. |
| 75 | 76 | CO-16 | Valsa mali |
| 77 | 78 | — | Pseudomonas sp. |
| 79 | 80 | — | Pseudomonas brassicacearum |
| 81 | 82 | CO-17 | Coniochaeta pulveracea |
| 83 | 84 | — | Pseudomonas ogarae |
| 85 | 86 | — | Pseudomonas fluorescens |
| 87 | 88 | — | Pseudomonas syringae |
| 89 | 90 | — | Pseudomonas sp. |
| 91 | 92 | — | Pseudomonas fluorescens |
| 93 | 94 | — | Pseudomonas fluorescens |
| 95 | 96 | — | Pseudomonas fluorescens |
| 97 | 98 | PbLSD | Pseudomonas brassicacearum |
| 99 | 100 | CO-18 | Diaporthe helianthi |
| 101 | 102 | — | Pseudomonas sp. |
| 103 | 104 | — | Pseudomonas sp. |
| 105 | 106 | — | Pseudomonas marginalis |
| 107 | 108 | — | Pseudomonas sp. |
| 109 | 110 | — | Pseudomonas sp. |
| 111 | 112 | — | Pseudomonas brassicacearum |
| 113 | 114 | CO-19 | Friedmanniomyces simplex |
| 115 | 116 | — | Pseudomonas brassicacearum |
| 117 | 118 | — | Pseudomonas sp. |
| 119 | 120 | CO-20 | Pseudomonas frederiksbergensis |
| 121 | 122 | — | Pseudomonas sp. |
| 123 | 124 | — | Pseudomonas kilonensis |
| 125 | 126 | CO-21 | Pseudomonas syringae |
| 127 | 128 | CO-22 | Pseudomonas kilonensis |
| 129 | 130 | CO-23 | Pseudomonas viciae |
| 131 | 132 | CO-24 | Pseudomonas ogarae |
| 133 | 134 | CO-25 (NOV1) | Novosphingobium aromaticivorans |
| | 135 | CO-25 (mutant | Artificial (derived from |
| | | S283F) | SEQ ID NO: 134) |

### DETAILED DESCRIPTION OF THE INVENTION

A first embodiment of the invention comprises an isolated eugenol oxidase capable of catalysing the reaction from eugenol (2-Methoxy-4-(prop-2-en-1-yl)phenol) to coniferyl alcohol (4-[(1E)-3-Hydroxyprop-1-en-1-yl]-2-methoxyphenol) in an aqueous medium comprising a solvent, eugenol oxidase and eugenol, and optionally an antioxidant agent, wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the eugenol have been converted to coniferyl alcohol.

The incubation time of the aqueous medium may be at least 0.5 h, at least 1 h, at least 1.5 h, at least 2 h, at least 2.5 h, at least 5 h, at least 10 h at least 12 h, or at least 24 h.

In one embodiment the incubation is performed at 10° C. to 50° C., preferably at 15° C. to 40° C., more preferably at 20° C. to 40° C., even more preferably at 24° C. to 37° C., most preferably at 28° C. to 32° C.

In a preferred embodiment after incubation for 10 hours at 30° C. at least 20% of the eugenol have been converted to coniferyl alcohol. In a more preferred embodiment after incubation for 7 hours at 30° C. at least 20% of the eugenol have been converted to coniferyl alcohol. In an even more preferred embodiment after incubation for 7 hours at 30° C. at least 25% of the eugenol have been converted to coniferyl alcohol. In an even more preferred embodiment after incubation for 18 h at 30° C. at least 40% of the eugenol have been converted to coniferyl alcohol. In a most preferred embodiment after incubation for 5 hours at 30° C. at least 25% of the eugenol have been converted to coniferyl alcohol.

The inventors of the present invention have also found out that the addition of an antioxidant agent leads to an improved catalytic reaction. The antioxidant agent can be a hydrophilic or lipophilic compound or an antioxidant enzyme. Examples of bioactive antioxidant compounds are ascorbic acid, glutathione, lipoic acid, uric acid, dithiothreitol, or carotenes. Examples of antioxidant enzymes are catalases, peroxidases, superoxide dismutases or enzymes of the thioredoxin or glutathione system.

In a preferred embodiment, the antioxidant agent is an enzyme. Preferably, said enzyme is a catalase.

In one embodiment, the isolated eugenol oxidase is comprising a sequence selected from the group consisting of
a. The amino acid molecule of SEQ ID NO: 2, 4, 6, 8, 10, 12, 14, 16, 18, 20, or 22 and
b. An amino acid molecule having at least 50% identity to the amino acid molecule of SEQ ID NO: 2, 4, 6, 8, 10, 12, 14, 16, 18, 20, or 22, or a functional fragment thereof, and
c. An amino acid molecule encoded by a nucleic acid molecule of SEQ ID NO: 1, 3, 5, 7, 9, 11, 13, 15, 17, 19, or 21, or a functional fragment thereof, and
d. An amino acid molecule encoded by a nucleic acid molecule having at least 50% identity to SEQ ID NO: 1, 3, 5, 7, 9, 11, 13, 15, 17, 19, or 21, or a functional fragment thereof, and
e. An amino acid molecule encoded by a nucleic acid molecule hybridizing under stringent conditions to a fragment of at least 250 bases complementary to SEQ ID NO: 1, 3, 5, 7, 9, 11, 13, 15, 17, 19, or 21, or a functional fragment thereof,
   wherein the amino acid molecule as defined in b., c., d. and e. is catalysing the reaction from eugenol to coniferyl alcohol in an aqueous medium, and,
   wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the eugenol have been converted to coniferyl alcohol.

The incubation time of the aqueous medium may be at least 0.5 h, at least 1 h, at least 1.5 h, at least 2 h, at least 2.5 h, at least 5 h, at least 10 h at least 12 h, or at least 24 h.

In one embodiment the incubation is performed at 10° C. to 50° C., preferably at 15° C. to 40° C., more preferably at 20° C. to 40° C., even more preferably at 24° C. to 37° C., most preferably at 28° C. to 32° C.

In a preferred embodiment after incubation for 10 hours at 30° C. at least 20% of the eugenol have been converted to coniferyl alcohol. In a more preferred embodiment after incubation for 7 hours at 30° C. at least 20% of the eugenol have been converted to coniferyl alcohol. In an even more preferred embodiment after incubation for 7 hours at 30° C. at least 25% of the eugenol have been converted to coniferyl alcohol. In an even more preferred embodiment after incubation for 18 h at 30° C. at least 40% of the eugenol have been converted to coniferyl alcohol. In a most preferred embodiment after incubation for 5 hours at 30° C. at least 25% of the eugenol have been converted to coniferyl alcohol.

A further embodiment of the invention is an isolated eugenol oxidase comprising a sequence selected from the group consisting of
a. The amino acid molecule of SEQ ID NO: 2, 4, 6, 8, 10, 12, 14, 16, 18, 20, or 22 and
b. An amino acid molecule having at least 50% identity to the amino acid molecule of SEQ ID NO: 2, 4, 6, 8, 10, 12, 14, 16, 18, 20, or 22, or a functional fragment thereof, and
c. An amino acid molecule encoded by a nucleic acid molecule of SEQ ID NO: 1, 3, 5, 7, 9, 11, 13, 15, 17, 19, or 21 or a functional fragment thereof, and
d. An amino acid molecule encoded by a nucleic acid molecule having at least 50% identity to SEQ ID NO: 1, 3, 5, 7, 9, 11, 13, 15, 17, 19, or 21 or a functional fragment thereof, and
e. An amino acid molecule encoded by a nucleic acid molecule hybridizing under stringent conditions to a fragment of at least 250 bases complementary to SEQ ID NO: 1, 3, 5, 7, 9, 11, 13, 15, 17, 19, or 21 or a functional fragment thereof,
   wherein the amino acid molecule as defined in b., c., d. and e. is catalysing the reaction from eugenol to coniferyl alcohol in an aqueous medium.

A further embodiment of the invention is a process for producing coniferyl alcohol comprising the steps of
i. Providing an aqueous medium comprising a solvent, one or more eugenol oxidase and eugenol and optionally an antioxidant agent and
ii. Incubating the aqueous medium and
iii. Optionally isolating the coniferyl alcohol from the reaction mixture,
   wherein the one or more eugenol oxidase is capable of catalysing the reaction from eugenol to coniferyl alcohol in an aqueous medium comprising a solvent, eugenol oxidase and eugenol and optionally an antioxidant agent, wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the eugenol have been converted to coniferyl alcohol.

The incubation time of the aqueous medium may be at least 0.5 h, at least 1 h, at least 1.5 h, at least 2 h, at least 2.5 h, at least 5 h, at least 10 h at least 12 h, or at least 24 h.

In one embodiment the incubation is performed at 10° C. to 50° C., preferably at 15° C. to 40° C., more preferably at 20° C. to 40° C., even more preferably at 24° C. to 37° C., most preferably at 28° C. to 32° C.

In a preferred embodiment after incubation for 10 hours at 30° C. at least 20% of the eugenol have been converted to coniferyl alcohol. In a more preferred embodiment after incubation for 7 hours at 30° C. at least 20% of the eugenol have been converted to coniferyl alcohol. In an even more preferred embodiment after incubation for 7 hours at 30° C. at least 25% of the eugenol have been converted to coniferyl alcohol. In an even more preferred embodiment after incubation for 18 h at 30° C. at least 40% of the eugenol have been converted to coniferyl alcohol. In a most preferred embodiment after incubation for 5 hours at 30° C. at least 25% of the eugenol have been converted to coniferyl alcohol.

The aqueous medium may be a solution or a suspension or a solution and a suspension, wherein any of the substances comprised in said aqueous medium may be fully or partially dissolved and/or partially or fully suspended.

In one embodiment of the process of the invention the eugenol oxidase is comprising a sequence selected from the group consisting of
a. The amino acid molecule of SEQ ID NO: 2, 4, 6, 8, 10, 12, 14, 16, 18, 20, or 22 and
b. An amino acid molecule having at least 50% identity to the amino acid molecule of SEQ ID NO: 2, 4, 6, 8, 10, 12, 14, 16, 18, 20, or 22, or a functional fragment thereof, and
c. An amino acid molecule encoded by a nucleic acid molecule of SEQ ID NO: 1, 3, 5, 7, 9, 11, 13, 15, 17, 19, or 21 or a functional fragment thereof, and
d. An amino acid molecule encoded by a nucleic acid molecule having at least 50% identity to SEQ ID NO: 1, 3, 5, 7, 9, 11, 13, 15, 17, 19, or 21 or a functional fragment thereof, and
e. An amino acid molecule encoded by a nucleic acid molecule hybridizing under stringent conditions to a fragment of at least 250 bases complementary to SEQ ID NO: 1, 3, 5, 7, 9, 11, 13, 15, 17, 19, or 21 or a functional fragment thereof,
   wherein the amino acid molecule as defined in b., c., d. and e. is catalysing the reaction from eugenol to coniferyl alcohol in an aqueous medium and wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the eugenol have been converted to coniferyl alcohol.

The incubation time of the aqueous medium may be at least 0.5 h, at least 1 h, at least 1.5 h, at least 2 h, at least 2.5 h, at least 5 h, at least 10 h at least 12 h, or at least 24 h.

In one embodiment the incubation is performed at 10° C. to 50° C., preferably at 15° C. to 40° C., more preferably at 20° C. to 40° C., even more preferably at 24° C. to 37° C., most preferably at 28° C. to 32° C.

In a preferred embodiment after incubation for 10 hours at 30° C. at least 20% of the eugenol have been converted to coniferyl alcohol. In a more preferred embodiment after incubation for 7 hours at 30° C. at least 20% of the eugenol have been converted to coniferyl alcohol. In an even more preferred embodiment after incubation for 7 hours at 30° C. at least 25% of the eugenol have been converted to coniferyl alcohol. In an even more preferred embodiment after incubation for 18 h at 30° C. at least 40% of the eugenol have been converted to coniferyl alcohol.

In a most preferred embodiment after incubation for 5 hours at 30° C. at least 25% of the eugenol have been converted to coniferyl alcohol.

A further embodiment of the invention is a process for producing coniferyl alcohol comprising the steps of
i. Providing an aqueous medium comprising a solvent, one or more eugenol oxidase and eugenol and optionally an antioxidant agent and,
ii. Incubating the aqueous medium and
iii. Optionally isolating the coniferyl alcohol from the reaction mixture,
   wherein the one or more eugenol oxidase is comprising a sequence selected from the group consisting of
a. The amino acid molecule of SEQ ID NO: 2, 4, 6, 8, 10, 12, 14, 16, 18, 20, or 22 and
b. An amino acid molecule having at least 50% identity to the amino acid molecule of SEQ ID NO: 2, 4, 6, 8, 10, 12, 14, 16, 18, 20, or 22 or a functional fragment thereof, and
c. An amino acid molecule encoded by a nucleic acid molecule of SEQ ID NO: 1, 3, 5, 7, 9, 11, 13, 15, 17, 19, or 21 or a functional fragment thereof, and
d. An amino acid molecule encoded by a nucleic acid molecule having at least 50% identity to SEQ ID NO: 1, 3, 5, 7, 9, 11, 13, 15, 17, 19, or 21 or a functional fragment thereof, and
e. An amino acid molecule encoded by a nucleic acid molecule hybridizing under stringent conditions to a fragment of at least 250 bases complementary to SEQ ID NO: 1, 3, 5, 7, 9, 11, 13, 15, 17, 19, or 21 or a functional fragment thereof,
   wherein the amino acid molecule as defined in b., c., d. and e. is catalysing the reaction from eugenol to coniferyl alcohol in an aqueous medium.

The aqueous medium may be a solution or a suspension or emulsion/dispersion, wherein any of the substances comprised in said aqueous medium may be fully or partially dissolved and/or partially or fully suspended.

A further embodiment of the invention is a recombinant construct comprising a nucleic acid molecule encoding a eugenol oxidase wherein the eugenol oxidase is comprising a sequence encoding an amino acid molecule selected from the group consisting of
a. The amino acid molecule of SEQ ID NO: 2, 4, 6, 8, 10, 12, 14, 16, 18, 20, or 22 and
b. An amino acid molecule having at least 50% identity to the amino acid molecule of SEQ ID NO: 2, 4, 6, 8, 10, 12, 14, 16, 18, 20, or 22 or a functional fragment thereof, and
c. An amino acid molecule encoded by a nucleic acid molecule of SEQ ID NO: 1, 3, 5, 7, 9, 11, 13, 15, 17, 19, or 21 or a functional fragment thereof, and
d. An amino acid molecule encoded by a nucleic acid molecule having at least 50% identity to SEQ ID NO: 1, 3, 5, 7, 9, 11, 13, 15, 17, 19, or 21 or a functional fragment thereof, and
e. An amino acid molecule encoded by a nucleic acid molecule hybridizing under stringent conditions to a fragment of at least 250 bases complementary to SEQ ID NO: 1, 3, 5, 7, 9, 11, 13, 15, 17, 19, or 21 or a functional fragment thereof,
   wherein the amino acid molecule as defined in b., c., d. and e. is catalysing the reaction from eugenol to coniferyl alcohol in an aqueous medium.

Said recombinant construct for expressing the nucleic acid encoding a eugenol oxidase may be integrated into the genome of an organism or the recombinant construct for expressing the nucleic acid encoding a eugenol oxidase may be comprised on a vector such as a plasmid or viral vector that is introduced into an organism.

The nucleic acid encoding a eugenol oxidase in the recombinant construct may be functionally linked to a heterologous promoter, a heterologous terminator and/or any other heterologous genetic element.

A further embodiment of the invention is a recombinant vector, such a s an expression vector or a viral vector comprising said recombinant construct.

A further embodiment of the invention is a recombinant microorganism comprising said recombinant construct or said recombinant vector.

In some embodiments, the recombinant microorganism is a prokaryotic cell. Suitable prokaryotic cells include Gram-positive, Gram negative and Gram-variable bacterial cells, preferably Gram-negative.

Thus, prokaryotic microorganisms that can be used in the present invention include, but are not limited to, Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium fiavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzer, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., Leptolyngbya sp.

Eukaryotic microorganisms that can be used in the present invention include, but are not limited to Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Klebsiella spec, such as Klebsiella pneumonia, Aspergillus spec. such as Aspergillus niger or Myceliophthora thermophila.

Preferred microorganisms of the invention comprise Rhodococcus rhodochrous, Aerococcus sp., Aspergillus sp., Bacillus pumilus, Bacillus subtilis, Bacteroides thetaiotaomicron, Clostridium algidicamis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica.

Especially preferred microorganisms are Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica.

A further embodiment of the invention is a composition comprising a solvent, a eugenol oxidase, eugenol, and optionally an antioxidant agent, wherein the eugenol oxidase is comprising a sequence selected from the group consisting of
a. The amino acid molecule of SEQ ID NO: 2, 4, 6, 8, 10, 12, 14, 16, 18, 20, or 22 and
b. An amino acid molecule having at least 50% identity to the amino acid molecule of SEQ ID NO: 2, 4, 6, 8, 10, 12, 14, 16, 18, 20, or 22 or a functional fragment thereof, and
c. An amino acid molecule encoded by a nucleic acid molecule of SEQ ID NO: 1, 3, 5, 7, 9, 11, 13, 15, 17, 19, or 21 or a functional fragment thereof, and
d. An amino acid molecule encoded by a nucleic acid molecule having at least 50% identity to SEQ ID NO: 1, 3, 5, 7, 9, 11, 13, 15, 17, 19, or 21 or a functional fragment thereof, and
e. An amino acid molecule encoded by a nucleic acid molecule hybridizing under stringent conditions to a fragment of at least 250 bases complementary to SEQ ID NO: 1, 3, 5, 7, 9, 11, 13, 15, 17, 19, or 21 or a functional fragment thereof,
   wherein the amino acid molecule as defined in b., c., d. and e. is catalysing the reaction from eugenol to coniferyl alcohol in an aqueous medium.

Another embodiment of the invention comprises an isolated alkene cleavage oxygenase (also known to the persons skilled in the art as isoeugenol monooxygenase, aromatic dioxygenase) capable of catalyzing the reaction from coniferyl alcohol (or isoeugenol; 4-[(1E)-3-Hydroxyprop-1-en-1-yl]-2-methoxyphenol) to vanillin (4-Hydroxy-3-methoxybenzaldehyde) in an aqueous medium comprising a solvent, alkene cleavage oxygenase and coniferyl alcohol, and optionally an iron(II) salt, and/or an antioxidant agent, wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the coniferyl alcohol have been converted to vanillin.

The incubation time of the aqueous medium may be at least 0.5 h, at least 1 h, at least 1.5 h, at least 2 h, at least 2.5 h, at least 5 h, at least 10 h, at least 12 h, or at least 24 h.

In one embodiment the incubation is performed at 10° C. to 50° C., preferably at 15° C. to 40° C., more preferably at 20° C. to 40° C., even more preferably at 24° C. to 37° C., most preferably at 28° C. to 32° C.

In a preferred embodiment after incubation for 10 hours at 30° C. at least 20% of the coniferyl alcohol/isoeugenol have been converted to vanillin. In a more preferred embodiment after incubation for 7 hours at 30° C. at least 20% of the coniferyl alcohol/isoeugenol have been converted to vanillin. In an even more preferred embodiment after incubation for 7 hours at 30° C. at least 25% of the coniferyl alcohol/isoeugenol have been converted to vanillin. In an even more preferred embodiment after incubation for 18 h at 30° C. at least 40% of the coniferyl alcohol/isoeugenol have been converted to vanillin. In a most preferred embodiment after incubation for 5 hours at 30° C. at least 25% of the coniferyl alcohol/isoeugenol have been converted to vanillin.

In one embodiment, the isolated alkene cleavage oxygenase is comprising a sequence selected from the group consisting of
a. The amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, and
b. An amino acid molecule having at least 50% identity to the amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, or a functional fragment thereof, and
c. An amino acid molecule encoded by a nucleic acid molecule of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
d. An amino acid molecule encoded by a nucleic acid molecule having at least 50% identity to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
e. An amino acid molecule encoded by a nucleic acid molecule hybridizing under stringent conditions to a fragment of at least 250 bases complementary to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof,
   wherein the amino acid molecule as defined in b., c., d. and e. is catalysing the reaction from coniferyl alcohol to vanillin in an aqueous medium, and,
   wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the coniferyl alcohol have been converted to vanillin.

The incubation time of the aqueous medium may be at least 0.5 h, at least 1 h, at least 1.5 h, at least 2 h, at least 2.5 h, at least 5 h, at least 10 h, at least 12 h, or at least 24 h.

In one embodiment the incubation is performed at 10° C. to 50° C., preferably at 15° C. to 40° C., more preferably at 20° C. to 40° C., even more preferably at 24° C. to 37° C., most preferably at 28° C. to 32° C.

In a preferred embodiment after incubation for 10 hours at 30° C. at least 20% of the coniferyl alcohol/isoeugenol have been converted to vanillin. In a more preferred embodiment after incubation for 7 hours at 30° C. at least 20% of the coniferyl alcohol/isoeugenol have been converted to vanillin. In an even more preferred embodiment after incubation for 7 hours at 30° C. at least 25% of the coniferyl alcohol/isoeugenol have been converted to vanillin. In an even more preferred embodiment after incubation for 18 h at 30° C. at least 40% of the coniferyl alcohol/isoeugenol have been converted to vanillin. In a most preferred embodiment after incubation for 5 hours at 30° C. at least 25% of the coniferyl alcohol/isoeugenol have been converted to vanillin.

A further embodiment of the invention is an isolated alkene cleavage oxygenase comprising a sequence selected from the group consisting of
a. The amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, and
b. An amino acid molecule having at least 50% identity to the amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, or a functional fragment thereof, and
c. An amino acid molecule encoded by a nucleic acid molecule of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof, and
d. An amino acid molecule encoded by a nucleic acid molecule having at least 50% identity to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof, and
e. An amino acid molecule encoded by a nucleic acid molecule hybridizing under stringent conditions to a fragment of at least 250 bases complementary to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof,
   wherein the amino acid molecule as defined in b., c., d. and e. is catalysing the reaction from coniferyl alcohol to vanillin in an aqueous medium.

A further embodiment of the invention is a process for producing vanillin comprising the steps of
i. Providing an aqueous medium comprising a solvent, one or more alkene cleavage oxygenase and coniferyl alcohol/isoeugenol and optionally an iron(II) salt, and/or an antioxidant agent and
ii. Incubating the aqueous medium and
iii. Optionally isolating the vanillin from the reaction mixture,
   wherein the one or more alkene cleavage oxygenase is capable of catalysing the reaction from coniferyl alcohol to vanillin in an aqueous medium comprising a solvent, alkene cleavage oxygenase and coniferyl alcohol and optionally an iron(II) salt, and/or an antioxidant agent, wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the coniferyl alcohol have been converted to vanillin.

The incubation time of the aqueous medium may be at least 0.5 h, at least 1 h, at least 1.5 h, at least 2 h, at least 2.5 h, at least 5 h, at least 10 h, at least 12 h, or at least 24 h.

In one embodiment the incubation is performed at 10° C. to 50° C., preferably at 15° C. to 40° C., more preferably at 20° C. to 40° C., even more preferably at 24° C. to 37° C., most preferably at 28° C. to 32° C.

In a preferred embodiment after incubation for 10 hours at 30° C. at least 20% of the coniferyl alcohol/isoeugenol have been converted to vanillin. In a more preferred embodiment after incubation for 7 hours at 30° C. at least 20% of the coniferyl alcohol/isoeugenol have been converted to vanillin. In an even more preferred embodiment after incubation for 7 hours at 30° C. at least 25% of the coniferyl alcohol/isoeugenol have been converted to vanillin. In an even more preferred embodiment after incubation for 18 h at 30° C. at least 40% of the coniferyl alcohol/isoeugenol have been converted to vanillin. In a most preferred embodiment after incubation for 5 hours at 30° C. at least 25% of the coniferyl alcohol/isoeugenol have been converted to vanillin.

The aqueous medium may be a solution or a suspension or a solution and a suspension, wherein any of the substances comprised in said aqueous medium may be fully or partially dissolved and/or partially or fully suspended.

In one embodiment of the process of the invention the alkene cleavage oxygenase is comprising a sequence selected from the group consisting of
a. The amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, and
b. An amino acid molecule having at least 50% identity to the amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, or a functional fragment thereof, and
c. An amino acid molecule encoded by a nucleic acid molecule of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof, and
d. An amino acid molecule encoded by a nucleic acid molecule having at least 50% identity to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof, and
e. An amino acid molecule encoded by a nucleic acid molecule hybridizing under stringent conditions to a fragment of at least 250 bases complementary to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof,
   wherein the amino acid molecule as defined in b., c., d. and e. is catalysing the reaction from coniferyl alcohol to vanillin in an aqueous medium and wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the coniferyl alcohol have been converted to vanillin.

The incubation time of the aqueous medium may be at least 0.5 h, at least 1 h, at least 1.5 h, at least 2 h, at least 2.5 h, at least 5 h, at least 10 h, at least 12 h, or at least 24 h.

In one embodiment the incubation is performed at 10° C. to 50° C., preferably at 15° C. to 40° C., more preferably at 20° C. to 40° C., even more preferably at 24° C. to 37° C., most preferably at 28° C. to 32° C.

In a preferred embodiment after incubation for 10 hours at 30° C. at least 20% of the coniferyl alcohol/isoeugenol have been converted to vanillin. In a more preferred embodiment after incubation for 7 hours at 30° C. at least 20% of the coniferyl alcohol/isoeugenol have been converted to vanillin. In an even more preferred embodiment after incubation for 7 hours at 30° C. at least 25% of the coniferyl alcohol/isoeugenol have been converted to vanillin. In an even more preferred embodiment after incubation for 18 h at 30° C. at least 40% of the coniferyl alcohol/isoeugenol have been converted to vanillin. In a most preferred embodiment after incubation for 5 hours at 30° C. at least 25% of the coniferyl alcohol/isoeugenol have been converted to vanillin.

A further embodiment of the invention is a process for producing vanillin comprising the steps of
i. Providing an aqueous medium comprising a solvent, one or more alkene cleavage oxygenase and coniferyl alcohol optionally an iron(II) salt, and/or an antioxidant agent, and,
ii. Incubating the aqueous medium and
iii. Optionally isolating the vanillin from the reaction mixture,
   wherein the one or more alkene cleavage oxygenase is comprising a sequence selected from the group consisting of
a. The amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, and
b. An amino acid molecule having at least 50% identity to the amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, or a functional fragment thereof, and
c. An amino acid molecule encoded by a nucleic acid molecule of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof, and
d. An amino acid molecule encoded by a nucleic acid molecule having at least 50% identity to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof, and
e. An amino acid molecule encoded by a nucleic acid molecule hybridizing under stringent conditions to a fragment of at least 250 bases complementary to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof,
   wherein the amino acid molecule as defined in b., c., d. and e. is catalysing the reaction from coniferyl alcohol to vanillin in an aqueous medium.

The aqueous medium may be a solution or a suspension or a solution and a suspension, wherein any of the substances comprised in said aqueous medium may be fully or partially dissolved and/or partially or fully suspended.

A further embodiment of the invention is a recombinant construct comprising a nucleic acid molecule encoding an alkene cleavage oxygenase wherein the alkene cleavage oxygenase is comprising a sequence encoding an amino acid molecule selected from the group consisting of
a. The amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, and
b. An amino acid molecule having at least 50% identity to the amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, or a functional fragment thereof, and
c. An amino acid molecule encoded by a nucleic acid molecule of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof, and
d. An amino acid molecule encoded by a nucleic acid molecule having at least 50% identity to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof, and
e. An amino acid molecule encoded by a nucleic acid molecule hybridizing under stringent conditions to a fragment of at least 250 bases complementary to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof,
   wherein the amino acid molecule as defined in b., c., d. and e. is catalysing the reaction from coniferyl alcohol to vanillin in an aqueous medium.

Said recombinant construct for expressing the nucleic acid encoding a alkene cleavage oxygenase may be integrated into the genome of an organism or the recombinant construct for expressing the nucleic acid encoding a alkene cleavage oxygenase may be comprised on a vector such as a plasmid or viral vector that is introduced into an organism.

The nucleic acid encoding the alkene cleavage oxygenase in the recombinant construct may be functionally linked to a heterologous promoter, a heterologous terminator and/or any other heterologous genetic element.

A further embodiment of the invention is a recombinant vector, such a s an expression vector or a viral vector comprising said recombinant construct.

A further embodiment of the invention is a recombinant microorganism comprising said recombinant construct or said recombinant vector.

In some embodiments, the recombinant microorganism is a prokaryotic cell. Suitable prokaryotic cells include Gram-positive, Gram negative and Gram-variable bacterial cells, preferably Gram-negative.

Thus, prokaryotic microorganisms that can be used in the present invention include, but are not limited to, Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., Leptolyngbya sp.

Eukaryotic microorganisms that can be used in the present invention include, but are not limited to Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Klebsiella spec, such as Klebsiella pneumonia, Aspergillus spec. such as Aspergillus niger or Myceliophthora thermophila.

Preferred microorganisms of the invention comprise Rhodococcus rhodochrous, Aerococcus sp., Aspergillus sp., Bacillus pumilus, Bacillus subtilis, Bacteroides thetaiotaomicron, Clostridium algidicamis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica.

Especially preferred microorganisms are Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica.

A further embodiment of the invention is a composition comprising a solvent, a alkene cleavage oxygenase, coniferyl alcohol, and optionally an iron(II) salt, and/or an antioxidant agent wherein the alkene cleavage oxygenase is comprising a sequence selected from the group consisting of
a. The amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, and
b. An amino acid molecule having at least 50% identity to the amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, or a functional fragment thereof, and
c. An amino acid molecule encoded by a nucleic acid molecule of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof, and
d. An amino acid molecule encoded by a nucleic acid molecule having at least 50% identity to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof, and
e. An amino acid molecule encoded by a nucleic acid molecule hybridizing under stringent conditions to a fragment of at least 250 bases complementary to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133 or a functional fragment thereof,
   wherein the amino acid molecule as defined in b., c., d. and e. is catalysing the reaction from coniferyl alcohol to vanillin in an aqueous medium.

A further embodiment of the invention is a process for producing vanillin comprising the steps of
i. Providing an aqueous medium comprising a solvent, one or more eugenol oxidase, one or more alkene cleavage oxygenase and eugenol and optionally an iron(II) salt, and/or an antioxidant agent and,
ii. Incubating the aqueous medium and
iii. Optionally isolating the vanillin from the reaction mixture,
   wherein the one or more eugenol oxidase is capable of catalysing the reaction from eugenol to coniferyl alcohol in an aqueous medium comprising a solvent, eugenol oxidase and eugenol, and
   wherein the one or more alkene cleavage oxygenase is capable of catalysing the reaction from coniferyl alcohol to vanillin in an aqueous medium comprising a solvent, alkene cleavage oxygenase and coniferyl alcohol, and optionally an iron(II) salt, and/or an antioxidant agent+, and
   wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the eugenol have been converted to vanillin.

The incubation time of the aqueous medium may be at least 0.5 h, at least 1 h, at least 1.5 h, at least 2 h, at least 2.5 h, at least 5 h, at least 10 h, at least 12 h, or at least 24 h.

In one embodiment the incubation is performed at 10° C. to 50° C., preferably at 15° C. to 40° C., more preferably at 20° C. to 40° C., even more preferably at 24° C. to 37° C., most preferably at 28° C. to 32° C.

In a preferred embodiment after incubation for 10 hours at 30° C. at least 20% of the eugenol have been converted to vanillin. In a more preferred embodiment after incubation for 7 hours at 30° C. at least 20% of the eugenol have been converted to vanillin. In an even more preferred embodiment after incubation for 7 hours at 30° C. at least 25% of the eugenol have been converted to vanillin. In an even more preferred embodiment after incubation for 18 h at 30° C. at least 40% of the eugenol have been converted to vanillin. In a most preferred embodiment after incubation for 5 hours at 30° C. at least 25% of the eugenol have been converted to vanillin.

The aqueous medium may be a solution or a suspension or a solution and a suspension, wherein any of the substances comprised in said aqueous medium may be fully or partially dissolved and/or partially or fully suspended.

In one embodiment of the process of the invention for producing vanillin, the eugenol oxidase is comprising a sequence selected from the group consisting of
a. The amino acid molecule of SEQ ID NO: 2, 4, 6, 8, 10, 12, 14, 16, 18, 20, or 22 and
b. An amino acid molecule having at least 50% identity to the amino acid molecule of SEQ ID NO: 2, 4, 6, 8, 10, 12, 14, 16, 18, 20, or 22 or a functional fragment thereof, and
c. An amino acid molecule encoded by a nucleic acid molecule of SEQ ID NO: 1, 3, 5, 7, 9, 11, 13, 15, 17, 19, or 21 or a functional fragment thereof, and
d. An amino acid molecule encoded by a nucleic acid molecule having at least 50% identity to SEQ ID NO: 1, 3, 5, 7, 9, 11, 13, 15, 17, 19, or 21 or a functional fragment thereof, and
e. An amino acid molecule encoded by a nucleic acid molecule hybridizing under stringent conditions to a fragment of at least 250 bases complementary to SEQ ID NO: 1, 3, 5, 7, 9, 11, 13, 15, 17, 19, or 21 or a functional fragment thereof,
   wherein the amino acid molecule as defined in b., c., d. and e. is catalysing the reaction from eugenol to coniferyl alcohol in an aqueous medium, wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the eugenol have been converted to vanillin.

The incubation time of the aqueous medium may be at least 0.5 h, at least 1 h, at least 1.5 h, at least 2 h, at least 2.5 h, at least 5 h, at least 10 h, at least 12 h, or at least 24 h.

In one embodiment the incubation is performed at 10° C. to 50° C., preferably at 15° C. to 40° C., more preferably at 20° C. to 40° C., even more preferably at 24° C. to 37° C., most preferably at 28° C. to 32° C.

In a preferred embodiment after incubation for 24 hours at 30° C. at least 30% of the eugenol have been converted to vanillin.

In a more preferred embodiment after incubation for 7 hours at 30° C. at least 20% of the eugenol have been converted to vanillin. In an even more preferred embodiment after incubation for 7 hours at 30° C. at least 25% of the eugenol have been converted to vanillin. In an even more preferred embodiment after incubation for 18 h at 30° C. at least 40% of the eugenol have been converted to vanillin. In a most preferred embodiment after incubation for 5 hours at 30° C. at least 25% of the eugenol have been converted to vanillin.

In a further process of the invention for producing vanillin the alkene cleavage oxygenase is comprising a sequence selected from the group consisting of
f. The amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, and
g. An amino acid molecule having at least 50% identity to the amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, or a functional fragment thereof, and
h. An amino acid molecule encoded by a nucleic acid molecule of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
i. An amino acid molecule encoded by a nucleic acid molecule having at least 50% identity to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
j. An amino acid molecule encoded by a nucleic acid molecule hybridizing under stringent conditions to a fragment of at least 250 bases complementary to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof,
   wherein the amino acid molecule as defined in g., h., i. and j. is catalysing the reaction from coniferyl alcohol to vanillin in an aqueous medium, wherein after incubation at least 10%, preferably at least 15%, more preferably at least 20%, even more preferably at least 25%, even more preferably at least 30%, even more preferably at least 35%, even more preferably at least 40%, even more preferably at least 45%, even more preferably at least 50%, even more preferably at least 55%, even more preferably at least 60%, even more preferably at least 65%, even more preferably at least 70%, even more preferably at least 75%, even more preferably at least 80%, even more preferably at least 85%, even more preferably at least 90%, even more preferably at least 95%, even more preferably at least 96%, 97%, 98%, 99% or 100% of the eugenol have been converted to vanillin.

The incubation time of the aqueous medium may be at least 0.5 h, at least 1 h, at least 1.5 h, at least 2 h, at least 2.5 h, at least 5 h, at least 10 h at least 12 h, or at least 24 h.

In one embodiment the incubation is performed at 10° C. to 50° C., preferably at 15° C. to 40° C., more preferably at 20° C. to 40° C., even more preferably at 24° C. to 30° C., most preferably at 28° C. to 32° C.

In a preferred embodiment after incubation for 10 hours at 30° C., at least 20% of the eugenol have been converted to vanillin. In a more preferred embodiment after incubation for 7 hours at 30° C. at least 20% of the eugenol have been converted to vanillin. In an even more preferred embodiment after incubation for 7 hours at 30° C. at least 25% of the eugenol have been converted to vanillin. In an even more preferred embodiment after incubation for 18 h at 30° C. at least 40% of the eugenol have been converted to vanillin. In a most preferred embodiment after incubation for 5 hours at 30° C. at least 25% of the eugenol have been converted to vanillin.

An additional embodiment of the invention is a process for producing vanillin comprising the steps of
i. Providing an aqueous medium comprising a solvent, one or more eugenol oxidase, one or more alkene cleavage oxygenase and eugenol and optionally an iron(II) salt, and/or an antioxidant agent and,
ii. Incubating the aqueous medium and
iii. Optionally isolating the vanillin from the reaction mixture,
   wherein the one or more eugenol oxidase is selected from the group consisting of
a. The amino acid molecule of SEQ ID NO: 2, 4, 6, 8, 10, 12, 14, 16, 18, 20, or 22 and
b. An amino acid molecule having at least 50% identity to the amino acid molecule of SEQ ID NO: 2, 4, 6, 8, 10, 12, 14, 16, 18, 20, or 22, or a functional fragment thereof, and
c. An amino acid molecule encoded by a nucleic acid molecule of SEQ ID NO: 1, 3, 5, 7, 9, 11, 13, 15, 17, 19, or 21 or a functional fragment thereof, and
d. An amino acid molecule encoded by a nucleic acid molecule having at least 50% identity to SEQ ID NO: 1, 3, 5, 7, 9, 11, 13, 15, 17, 19, or 21 or a functional fragment thereof, and
e. An amino acid molecule encoded by a nucleic acid molecule hybridizing under stringent conditions to a fragment of at least 250 bases complementary to SEQ ID NO: 1, 3, 5, 7, 9, 11, 13, 15, 17, 19, or 21 or a functional fragment thereof,
   wherein the amino acid molecule as defined in b., c., d. and e. is catalysing the reaction from eugenol to coniferyl alcohol in an aqueous medium.

In a further process of the invention for producing vanillin and the alkene cleavage oxygenase is comprising a sequence selected from the group consisting of
f. The amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, and
g. An amino acid molecule having at least 50% identity to the amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, or a functional fragment thereof, and
h. An amino acid molecule encoded by a nucleic acid molecule of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
i. An amino acid molecule encoded by a nucleic acid molecule having at least 50% identity to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
j. An amino acid molecule encoded by a nucleic acid molecule hybridizing under stringent conditions to a fragment of at least 250 bases complementary to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof,
   wherein the amino acid molecule as defined in g., h., i. and j. is catalysing the reaction from coniferyl alcohol to vanillin in an aqueous medium.

In a preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 30, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 24, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 26, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 38, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 40, or a functional variant thereof

The aqueous medium may be a solution or a suspension or a solution and a suspension, wherein any of the substances comprised in said aqueous medium may be fully or partially dissolved and/or partially or fully suspended.

A further embodiment of the invention is a recombinant construct comprising a nucleic acid encoding a eugenol oxidase and a nucleic acid encoding a alkene cleavage oxygenase wherein the eugenol oxidase is comprising a sequence encoding an amino acid molecule selected from the group consisting of
a. The amino acid molecule of SEQ ID NO: 2, 4, 6, 8, 10, 12, 14, 16, 18, 20, or 22 and
b. An amino acid molecule having at least 50% identity to the amino acid molecule of SEQ ID NO: 2, 4, 6, 8, 10, 12, 14, 16, 18, 20, or 22, or a functional fragment thereof, and
c. An amino acid molecule encoded by a nucleic acid molecule of SEQ ID NO: 1, 3, 5, 7, 9, 11, 13, 15, 17, 19, or 21 or a functional fragment thereof, and
d. An amino acid molecule encoded by a nucleic acid molecule having at least 50% identity to SEQ ID NO: 1, 3, 5, 7, 9, 11, 13, 15, 17, 19, or 21 or a functional fragment thereof, and
e. An amino acid molecule encoded by a nucleic acid molecule hybridizing under stringent conditions to a fragment of at least 250 bases complementary to SEQ ID NO: 1, 3, 5, 7, 9, 11, 13, 15, 17, 19, or 21 or a functional fragment thereof,
   wherein the amino acid molecule as defined in b., c., d. and e. is catalysing the reaction from eugenol to coniferyl alcohol in an aqueous medium.

In one embodiment of the recombinant construct comprising a nucleic acid encoding a eugenol oxidase and a nucleic acid encoding a alkene cleavage oxygenase, the alkene cleavage oxygenase is comprising a sequence encoding an amino acid molecule selected from the group consisting of
f. The amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, and
g. An amino acid molecule having at least 50% identity to the amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, or a functional fragment thereof, and
h. An amino acid molecule encoded by a nucleic acid molecule of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
i. An amino acid molecule encoded by a nucleic acid molecule having at least 50% identity to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
j. An amino acid molecule encoded by a nucleic acid molecule hybridizing under stringent conditions to a fragment of at least 250 bases complementary to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof,
   wherein the amino acid molecule as defined in g., h., i. and j. is catalysing the reaction from coniferyl alcohol to vanillin in an aqueous medium.

In a preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 30, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 24, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 26, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 38, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 40, or a functional variant thereof.

In a further embodiment of the recombinant construct comprising a nucleic acid encoding a eugenol oxidase and a nucleic acid encoding a alkene cleavage oxygenase each the nucleic acid encoding a eugenol oxidase and the nucleic acid encoding a alkene cleavage oxygenase are functionally linked to a heterologous regulatory element, for example a promoter, a terminator, an enhancer or any other heterologous element.

Another embodiment of the invention is a recombinant vector comprising the recombinant construct comprising a nucleic acid encoding a eugenol oxidase and a nucleic acid encoding a alkene cleavage oxygenase wherein each the nucleic acid encoding a eugenol oxidase and the nucleic acid encoding a alkene cleavage oxygenase are functionally linked to a heterologous for example a promoter, a terminator, an enhancer or any other heterologous element.

Another embodiment of the invention is a recombinant microorganism comprising a recombinant construct comprising a nucleic acid encoding a eugenol oxidase and a nucleic acid encoding a alkene cleavage oxygenase wherein each the nucleic acid encoding a eugenol oxidase and the nucleic acid encoding a alkene cleavage oxygenase are functionally linked to a heterologous regulatory element or comprising the recombinant vector comprising said recombinant construct.

The recombinant microorganism comprising a recombinant construct comprising a eugenol oxidase and a alkene cleavage oxygenase wherein each the eugenol oxidase and the alkene cleavage oxygenase are functionally linked to a heterologous regulatory element or comprising the recombinant vector comprising said recombinant construct is preferably selected from the list comprising, Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., Leptolyngbya sp., Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Klebsiella spec, such as Klebsiella pneumonia, Aspergillus spec. such as Aspergillus niger or Myceliophthora thermophila.

More preferably the recombinant microorganism is Rhodococcus rhodochrous, Aerococcus sp., Aspergillus sp., Bacillus pumilus, Bacillus subtilis, Bacteroides thetaiotaomicron, Clostridium algidicamis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica.

Especially preferred microorganisms are Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica.

Another embodiment of the invention is a composition comprising a solvent, one or more eugenol oxidase, one or more alkene cleavage oxygenase, eugenol and optionally an iron(II) salt, and/or an antioxidant agent wherein the eugenol oxidase is selected from the group consisting of
a. The amino acid molecule of SEQ ID NO: 2, 4, 6, 8, 10, 12, 14, 16, 18, 20, or 22 and
b. An amino acid molecule having at least 50% identity to the amino acid molecule of SEQ ID NO: 2, 4, 6, 8, 10, 12, 14, 16, 18, 20, or 22, or a functional fragment thereof, and
c. An amino acid molecule encoded by a nucleic acid molecule of SEQ ID NO: 1, 3, 5, 7, 9, 11, 13, 15, 17, 19, or 21 or a functional fragment thereof, and
d. An amino acid molecule encoded by a nucleic acid molecule having at least 50% identity to SEQ ID NO: 1, 3, 5, 7, 9, 11, 13, 15, 17, 19, or 21 or a functional fragment thereof, and
e. An amino acid molecule encoded by a nucleic acid molecule hybridizing under stringent conditions to a fragment of at least 250 bases complementary to SEQ ID NO: 1, 3, 5, 7, 9, 11, 13, 15, 17, 19, or 21 or a functional fragment thereof,
   wherein the amino acid molecule as defined in b., c., d. and e. is catalysing the reaction from eugenol to coniferyl alcohol in an aqueous medium.

In a further embodiment of the composition comprising a solvent, one or more eugenol oxidase, one or more alkene cleavage oxygenase, eugenol and optionally an iron(II) salt, and/or an antioxidant agent the alkene cleavage oxygenase is selected from the group consisting of
f. The amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, and
g. An amino acid molecule having at least 50% identity to the amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, or a functional fragment thereof, and
h. An amino acid molecule encoded by a nucleic acid molecule of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
i. An amino acid molecule encoded by a nucleic acid molecule having at least 50% identity to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
j. An amino acid molecule encoded by a nucleic acid molecule hybridizing under stringent conditions to a fragment of at least 250 bases complementary to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof,
   wherein the amino acid molecule as defined in g., h., i. and j. is catalysing the reaction from coniferyl alcohol to vanillin in an aqueous medium.

In a preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 30, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 24, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 26, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 38, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 40, or a functional variant thereof.

Another embodiment of the invention is a recombinant microorganism comprising an introduced, increased or enhanced activity and/or expression of one or more eugenol oxidase, one or more alkene cleavage oxygenase,
wherein the one or more eugenol oxidase is capable of catalysing the reaction from eugenol to coniferyl alcohol, and
wherein the one or more alkene cleavage oxygenase is capable of catalysing the reaction from coniferyl alcohol to vanillin, and
wherein the eugenol oxidase is comprising a sequence selected from the group consisting of
a. The amino acid molecule of SEQ ID NO: 2, 4, 6, 8, 10, 12, 14, 16, 18, 20, or 22 and
b. An amino acid molecule having at least 50% identity to the amino acid molecule of SEQ ID NO: 2, 4, 6, 8, 10, 12, 14, 16, 18, 20, or 22, or a functional fragment thereof, and
c. An amino acid molecule encoded by a nucleic acid molecule of SEQ ID NO: 1, 3, 5, 7, 9, 11, 13, 15, 17, 19, or 21 or a functional fragment thereof, and
d. An amino acid molecule encoded by a nucleic acid molecule having at least 50% identity to SEQ ID NO: 1, 3, 5, 7, 9, 11, 13, 15, 17, 19, or 21 or a functional fragment thereof, and
e. An amino acid molecule encoded by a nucleic acid molecule hybridizing under stringent conditions to a fragment of at least 250 bases complementary to SEQ ID NO: 1, 3, 5, 7, 9, 11, 13, 15, 17, 19, or 21 or a functional fragment thereof, and
   wherein the amino acid molecule as defined in b., c., d. and e. is catalysing the reaction from eugenol to coniferyl alcohol.

In a further embodiment of the recombinant microorganism comprising an introduced, increased or enhanced activity and/or expression of one or more eugenol oxidase, one or more alkene cleavage oxygenase, the alkene cleavage oxygenase is comprising a sequence selected from the group consisting of
f. The amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, and
g. An amino acid molecule having at least 50% identity to the amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, or a functional fragment thereof, and
h. An amino acid molecule encoded by a nucleic acid molecule of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
i. An amino acid molecule encoded by a nucleic acid molecule having at least 50% identity to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
j. An amino acid molecule encoded by a nucleic acid molecule hybridizing under stringent conditions to a fragment of at least 250 bases complementary to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and wherein the amino acid molecule as defined in g., h., i. and j. is catalysing the reaction from coniferyl alcohol to vanillin.

Another embodiment of the invention is a method for fermentative production of vanillin comprising the steps of
i. Providing a recombinant microorganism comprising an introduced, increased or enhanced activity and/or expression of one or more eugenol oxidase, one or more alkene cleavage oxygenase,
ii. Culturing said microorganism in a medium comprising eugenol under conditions that allow for the production of vanillin and optionally isolating said vanillin from the medium.

A further embodiment of the invention is a composition comprising one or more recombinant microorganisms comprising an introduced, increased or enhanced activity and/or expression of one or more eugenol oxidase, one or more alkene cleavage oxygenase.

In one embodiment, said composition is further comprising eugenol, a medium and a carbon source.

A further embodiment of the invention is a method for producing a recombinant microorganism comprising an introduced, increased or enhanced activity and/or expression of one or more eugenol oxidase, an introduced, increased or enhanced activity and/or expression of one or more alkene cleavage oxygenase, said method comprising the steps of:
(I) introducing, increasing or enhancing the activity and/or expression of a eugenol oxidase gene encoding a eugenol oxidase enzyme having a eugenol oxido-reducing activity in said microorganism; and
(II) introducing, increasing or enhancing the activity and/or expression of a alkene cleavage oxygenase gene encoding a alkene cleavage oxygenase enzyme having an coniferyl alcohol oxido-reducing activity in said microorganism;

The recombinant microorganism produced according to the method as defined above or used in the method for fermentative production of vanillin is preferably selected from the list comprising Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzer, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., Leptolyngbya sp., Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Klebsiella spec, such as Klebsiella pneumonia, Aspergillus spec. such as Aspergillus niger or Myceliophthora thermophila.

More preferably the recombinant microorganism is Rhodococcus rhodochrous, Aerococcus sp., Aspergillus sp., Bacillus pumilus, Bacillus subtilis, Bacteroides thetaiotaomicron, Clostridium algidicamis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica.

Especially preferred recombinant microorganisms are Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica.

A further embodiment of the invention is a recombinant expression construct comprising
i. a promoter functional in a microorganism functionally linked to a nucleic acid molecule encoding a eugenol oxidase and
ii. a promoter functional in a microorganism functionally linked to a nucleic acid molecule encoding alkene cleavage oxygenase,
   wherein at least one of the promoters functionally linked to the nucleic acid molecule encoding the eugenol oxidase or nucleic acid molecule encoding the alkene cleavage oxygenase is heterologous to the nucleic acid molecule encoding the eugenol oxidase or the nucleic acid molecule encoding the alkene cleavage oxygenase,
   wherein the eugenol oxidase is comprising a sequence encoding an amino acid molecule selected from the group consisting of
a. The amino acid molecule of SEQ ID NO: 2, 4, 6, 8, 10, 12, 14, 16, 18, 20, or 22 and
b. An amino acid molecule having at least 50% identity to the amino acid molecule of SEQ ID NO: 2, 4, 6, 8, 10, 12, 14, 16, 18, 20, or 22 or a functional fragment thereof, and
c. An amino acid molecule encoded by a nucleic acid molecule of SEQ ID NO: 1, 3, 5, 7, 9, 11, 13, 15, 17, 19, or 21 or a functional fragment thereof, and
d. An amino acid molecule encoded by a nucleic acid molecule having at least 50% identity to SEQ ID NO: 1, 3, 5, 7, 9, 11, 13, 15, 17, 19, or 21 or a functional fragment thereof, and
e. An amino acid molecule encoded by a nucleic acid molecule hybridizing under stringent conditions to a fragment of at least 250 bases complementary to SEQ ID NO: 1, 3, 5, 7, 9, 11, 13, 15, 17, 19, or 21 or a functional fragment thereof, and
   wherein the amino acid molecule as defined in b., c., d. and e. is catalysing the reaction from eugenol to coniferyl alcohol.

In one embodiment of the recombinant expression construct comprising
i. a promoter functional in a microorganism functionally linked to a nucleic acid molecule encoding a eugenol oxidase and
ii. a promoter functional in a microorganism functionally linked to a nucleic acid molecule encoding alkene cleavage oxygenase,
   wherein at least one of the promoters functionally linked to the nucleic acid molecule encoding the eugenol oxidase or nucleic acid molecule encoding the alkene cleavage oxygenase is heterologous to the nucleic acid molecule encoding the eugenol oxidase or nucleic acid molecule encoding the alkene cleavage oxygenase, the alkene cleavage oxygenase is comprising a sequence selected from the group consisting of
f. The amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, and
g. An amino acid molecule having at least 50% identity to the amino acid molecule of SEQ ID NO: 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, or a functional fragment thereof, and
h. An amino acid molecule encoded by a nucleic acid molecule of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
i. An amino acid molecule encoded by a nucleic acid molecule having at least 50% identity to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and
j. An amino acid molecule encoded by a nucleic acid molecule hybridizing under stringent conditions to a fragment of at least 250 bases complementary to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, or a functional fragment thereof, and wherein the amino acid molecule as defined in g., h., i. and j. is catalysing the reaction from coniferyl alcohol to vanillin.

In a preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 30, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 24, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 26, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 38, or a functional variant thereof

In another preferred embodiment, eugenol oxidase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, or a functional variant thereof, and
alkene cleavage oxygenase comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 40, or a functional variant thereof

The eugenol oxidase and alkene cleavage oxygenase may each be under control of a heterologous promoter or may be arranged in an operon under control of one promoter heterologous to the eugenol oxidase, alkene cleavage oxygenase or both. The operon may comprise further genes necessary for the production of vanillin.

Amino acid molecules having a certain identity to any of the sequences of SEQ ID NO 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135, include amino acid molecules having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to any of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135.

Nucleic acid molecules having a certain identity to any of the sequences of SEQ ID NO 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133, include nucleic acid molecules having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to any of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133.

A functional fragment of an amino acid molecule of the invention comprises at least 50 consecutive amino acids, preferably at least 75 consecutive amino acids, more preferably at least 100 consecutive amino acids, more preferably at least 125 consecutive amino acids, more preferably at least 150 consecutive amino acids, even more preferably at least 175 consecutive amino acids, even more preferably at least 200 consecutive amino acids, even more preferably at least 225 consecutive amino acids, most preferably at least 250 consecutive amino acids of any of the sequences of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 or 135.

A further embodiment of the invention is a recombinant vector comprising the recombinant expression construct comprising
i. a promoter functional in a microorganism functionally linked to a nucleic acid molecule encoding a eugenol oxidase and
ii. a promoter functional in a microorganism functionally linked to a nucleic acid molecule encoding alkene cleavage oxygenase,
   wherein at least one of the promoters functionally linked to the nucleic acid molecule encoding the eugenol oxidase or the alkene cleavage oxygenase is heterologous to the nucleic acid molecule encoding the eugenol oxidase or the alkene cleavage oxygenase. The nucleic acid molecule encoding the eugenol oxidase and the alkene cleavage oxygenase may each be under control of a heterologous promoter or may be arranged in an operon under control of one promoter heterologous to the nucleic acid molecule encoding the eugenol oxidase, alkene cleavage oxygenase or both. The operon may comprise further genes necessary for the production of vanillin.

A further embodiment of the invention is a recombinant microorganism comprising a) the recombinant expression construct comprising a promoter functional in a microorganism functionally linked to a nucleic acid molecule encoding a eugenol oxidase and a promoter functional in a microorganism functionally linked to a nucleic acid molecule encoding alkene cleavage oxygenase, wherein at least one of the promoters functionally linked to the nucleic acid molecule encoding the eugenol oxidase or the alkene cleavage oxygenase is heterologous to the nucleic acid molecule encoding the eugenol oxidase or alkene cleavage oxygenase or b) the recombinant vector comprising said recombinant expression construct.

Preferably the recombinant microorganism is Rhodococcus rhodochrous, Aerococcus sp., Aspergillus sp., Bacillus pumilus, Bacillus subtilis, Bacteroides thetaiotaomicron, Clostridium algidicamis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica.

Especially preferred recombinant microorganisms are Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica.

Another embodiment of the invention is a method of culturing or growing the recombinant microorganisms as defined above comprising inoculating a culture medium with one or more of said recombinant microorganisms and culturing or growing said recombinant microorganism in culture medium comprising eugenol.

Another embodiment of the invention is the use of the recombinant microorganism as defined above or the composition as defined above for the whole cell bio-conversion of eugenol to vanillin.

Another embodiment of the invention is a process for whole cell bio-conversion of eugenol to vanillin comprising the steps of
I) growing the recombinant microorganism as defined above in a fermenter comprising eugenol, a medium suitable for growing said recombinant microorganism and a C-source, and
II) recovering vanillin from the fermentation broth obtained in I).

Another embodiment of the invention is a process for whole cell bio-conversion of eugenol to vanillin comprising the step of
i) growing the recombinant microorganism as defined above in a fermenter comprising a medium suitable for growing said recombinant microorganism and a C-source, and
ii) recovering the recombinant microorganism from the fermenter, and
iii) performing a whole cell bio-conversion in a medium by supplementing eugenol, and
iv) recovering vanillin from the medium obtained in iii).

### Definitions

It is to be understood that this invention is not limited to the particular methodology or protocols.

It is also to be understood that the terminology used herein is for the purpose of describing particular embodiments only and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Antiparallel: “Antiparallel” refers herein to two nucleotide sequences paired through hydrogen bonds between complementary base residues with phosphodiester bonds running in the 5′-3′ direction in one nucleotide sequence and in the 3′-5′ direction in the other nucleotide sequence.

Antisense: The term “antisense” refers to a nucleotide sequence that is inverted relative to its normal orientation for transcription or function and so expresses an RNA transcript that is complementary to a target gene mRNA molecule expressed within the host cell (e.g., it can hybridize to the target gene mRNA molecule or single stranded genomic DNA through Watson-Crick base pairing) or that is complementary to a target DNA molecule such as, for example genomic DNA present in the host cell.

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

Donor DNA molecule: As used herein the terms “donor DNA molecule”, “repair DNA molecule” or “template DNA molecule” all used interchangeably herein mean a DNA molecule having a sequence that is to be introduced into the genome of a cell. It may be flanked at the 5′ and/or 3′ end by sequences homologous or identical to sequences in the target region of the genome of said cell. It may comprise sequences not naturally occurring in the respective cell such as ORFs, non-coding RNAs or regulatory elements that shall be introduced into the target region or it may comprise sequences that are homologous to the target region except for at least one mutation, a gene edit: The sequence of the donor DNA molecule may be added to the genome or it may replace a sequence in the genome of the length of the donor DNA sequence.

Double-stranded RNA: A “double-stranded RNA” molecule or “dsRNA” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule.

Endogenous: An “endogenous” nucleotide sequence refers to a nucleotide sequence, which is present in the genome of the untransformed cell.

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Expression construct: “Expression construct” as used herein means a DNA sequence capable of directing expression of a particular nucleotide sequence in a cell, comprising a promoter functional in said cell into which it will be introduced, operatively linked to the nucleotide sequence of interest which is—optionally—operatively linked to termination signals. If translation is required, it also typically comprises sequences required for proper translation of the nucleotide sequence. The coding region may code for a protein of interest but may also code for a functional RNA of interest, for example RNAa, siRNA, snoRNA, snRNA, microRNA, ta-siRNA or any other noncoding regulatory RNA, in the sense or antisense direction. The expression construct comprising the nucleotide sequence of interest may be chimeric, meaning that one or more of its components is heterologous with respect to one or more of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a constitutive promoter or of an inducible promoter, which initiates transcription only when the host cell is exposed to some particular external stimulus.

Foreign: The term “foreign” refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally-occurring sequence.

Functional linkage: The term “functional linkage” or “functionally linked” is to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfil its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc. and Wiley Interscience). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into a genome, for example by transformation.

Gene: The term “gene” refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

“Gene edit” when used herein means the introduction of a specific mutation at a specific position of the genome of a cell. The gene edit may be introduced by precise editing applying more advanced technologies e.g. using a CRISPR Cas system and a donor DNA, or a CRISPR Cas system linked to mutagenic activity such as a deaminase (WO15133554, WO17070632).

Genome and genomic DNA: The terms “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

Heterologous: The term “heterologous” with respect to a nucleic acid molecule or DNA refers to a nucleic acid molecule which is operably linked to, or is manipulated to become operably linked to, a second nucleic acid molecule, e.g. a promoter to which it is not operably linked in nature, e.g. in the genome of a WT cell, or to which it is operably linked at a different location or position in nature, e.g. in the genome of a WT cell.

Preferably the term “heterologous” with respect to a nucleic acid molecule or DNA refers to a nucleic acid molecule which is operably linked to, or is manipulated to become operably linked to, a second nucleic acid molecule, e.g. a promoter or an open reading frame to which it is not operably linked in nature.

A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example, a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

Hybridization: The term “hybridization” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:
DNA-DNA hybrids (Meinkoth and Wahl, Anal Biochem., 138: 267-284, 1984):

$$Tm = 81.5 °C . + 16.6 x log [ Na + ] a + 0.41 x \% [ G / Cb ] - 500 x [ Lc ] - 1 - 0.61 x \% formamide$$

DNA-RNA or RNA-RNA hybrids:

$$Tm = 79.8 + 18.5 ( log 10 [ Na + ] a ) + 0.58 ( \% G / Cb ) + 11.8 ( \% G / Cb ) 2 - 820 / Lc$$

oligo-DNA or oligo-RNAd hybrids:
For <20 nucleotides: Tm=2 (ln)
For 20-35 nucleotides: Tm=22+1.46 (ln)
a or for other monovalent cation, but only accurate in the 0.01-0.4 M range.
b only accurate for % GC in the 30% to 75% range.
c L=length of duplex in base pairs.
d Oligo, oligonucleotide; In, effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

“Identity”: “Identity” when used in respect to the comparison of two or more nucleic acid or amino acid molecules means that the sequences of said molecules share a certain degree of sequence similarity, the sequences being partially identical.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:
Seq A: AAGATACTG length: 9 bases
Seq B: GATCTGA length: 7 bases
Hence, the shorter sequence is sequence B.

Producing a pairwise global alignment which is showing both sequences over their complete lengths results in

| | |
|---|---|
| Seq A: | AAGATACTG- |
| | \|\|\| \|\|\| |
| Seq B: | --GAT-CTGA |

The “|” symbol in the alignment indicates identical residues (which means bases for DNA or amino acids for proteins). The number of identical residues is 6.

The - symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

| | |
|---|---|
| Seq A: | GATACTG- |
| | \|\|\| \|\|\| |
| Seq B: | GAT-CTGA |

Producing a pairwise alignment which is showing sequence A over its complete length according to the invention consequently results in:

| | |
|---|---|
| Seq A: | AAGATACTG |
| | \|\|\| \|\|\| |
| Seq B: | --GAT-CTG |

Producing a pairwise alignment which is showing sequence B over its complete length according to the invention consequently results in:

| | |
|---|---|
| Seq A: | GATACTG- |
| | \|\|\| \|\|\| |
| Seq B: | GAT-CTGA |

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”. According to the example provided above, %-identity is: for Seq A being the sequence of the invention (6/9)*100=66.7%; for Seq B being the sequence of the invention (6/8)*100=75%.

InDel is a term for the random insertion or deletion of bases in the genome of an organism associated with the repair of a DSB by NHEJ. It is classified among small genetic variations, measuring from 1 to 10 000 base pairs in length. As used herein it refers to random insertion or deletion of bases in or in the close vicinity (e.g. less than 1000 bp, 900 bp, 800 bp, 700 bp, 600 bp, 500 bp, 400 bp, 300 bp, 250 bp, 200 bp, 150 bp, 100 bp, 50 bp, 40 bp, 30 bp, 25 bp, 20 bp, 15 bp, 10 bp or 5 bp up and/or downstream) of the target site.

The term “Introducing”, “introduction” and the like with respect to the introduction of a donor DNA molecule in the target site of a target DNA means any introduction of the sequence of the donor DNA molecule into the target region for example by the physical integration of the donor DNA molecule or a part thereof into the target region or the introduction of the sequence of the donor DNA molecule or a part thereof into the target region wherein the donor DNA is used as template for a polymerase.

Intron: refers to sections of DNA (intervening sequences) within a gene that do not encode part of the protein that the gene produces, and that is spliced out of the mRNA that is transcribed from the gene before it is exported from the cell nucleus. Intron sequence refers to the nucleic acid sequence of an intron. Thus, introns are those regions of DNA sequences that are transcribed along with the coding sequence (exons) but are removed during the formation of mature mRNA. Introns can be positioned within the actual coding region or in either the 5′ or 3′ untranslated leaders of the pre-mRNA (unspliced mRNA). Introns in the primary transcript are excised and the coding sequences are simultaneously and precisely ligated to form the mature mRNA. The junctions of introns and exons form the splice site. The sequence of an intron begins with GU and ends with AG. Furthermore, in plants, two examples of AU-AC introns have been described: the fourteenth intron of the RecA-like protein gene and the seventh intron of the G5 gene from Arabidopsis thaliana are AT-AC introns. Pre-mRNAs containing introns have three short sequences that are—beside other sequences—essential for the intron to be accurately spliced. These sequences are the 5′ splice-site, the 3′ splice-site, and the branchpoint. mRNA splicing is the removal of intervening sequences (introns) present in primary mRNA transcripts and joining or ligation of exon sequences. This is also known as cis-splicing which joins two exons on the same RNA with the removal of the intervening sequence (intron).

The functional elements of an intron is comprising sequences that are recognized and bound by the specific protein components of the spliceosome (e.g. splicing consensus sequences at the ends of introns). The interaction of the functional elements with the spliceosome results in the removal of the intron sequence from the premature mRNA and the rejoining of the exon sequences. Introns have three short sequences that are essential—although not sufficient—for the intron to be accurately spliced. These sequences are the 5′ splice site, the 3′ splice site and the branch point. The branchpoint sequence is important in splicing and splice-site selection.

The branchpoint sequence is usually located 10-60 nucleotides upstream of the 3′ splice site.

Isogenic: organisms, which are genetically identical, except that they may differ by the presence or absence of a heterologous DNA sequence.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living cell is not isolated, but the same polynucleotide or polypeptide, separated from some or all of the coexisting materials in the natural system, is isolated. Such polynucleotides can be part of a vector and/or such polynucleotides or polypeptides could be part of a composition and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighbouring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 2 includes, by way of example, such nucleic acid sequences in cells which ordinarily contain SEQ ID NO:2 where the nucleic acid sequence is in a chromosomal or extrachromosomal location different from that of natural cells or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Minimal Promoter: promoter elements, particularly a TATA element, that are inactive or that have greatly reduced promoter activity in the absence of upstream activation. In the presence of a suitable transcription factor, the minimal promoter functions to permit transcription.

Non-coding: The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions.

Nucleic acids and nucleotides: The terms “Nucleic Acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form.

Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Pre-protein: Protein, which is normally targeted to a cellular organelle, such as a chloroplast, and still comprising its transit peptide.

“Precise” with respect to the introduction of a donor DNA molecule in target region means that the sequence of the donor DNA molecule is introduced into the target region without any InDels, duplications or other mutations as compared to the unaltered DNA sequence of the target region that are not comprised in the donor DNA molecule sequence.

Primary transcript: The term “primary transcript” as used herein refers to a premature RNA transcript of a gene. A “primary transcript” for example still comprises introns and/or is not yet comprising a polyA tail or a cap structure and/or is missing other modifications necessary for its correct function as transcript such as for example trimming or editing.

Promoter: The terms “promoter”, or “promoter sequence” are equivalents and as used herein, refer to a DNA sequence which when ligated to a nucleotide sequence of interest is capable of controlling the transcription of the nucleotide sequence of interest into RNA. A promoter is located 5′ (i.e., upstream), proximal to the transcriptional start site of a nucleotide sequence of interest whose transcription into mRNA it controls and provides a site for specific binding by RNA polymerase and other transcription factors for initiation of transcription. Said promoter comprises for example the at least 10 kb, for example 5 kb or 2 kb proximal to the transcription start site. It may also comprise the at least 1500 bp proximal to the transcriptional start site, preferably the at least 1000 bp, more preferably the at least 500 bp, even more preferably the at least 400 bp, the at least 300 bp, the at least 200 bp or the at least 100 bp. In a further preferred embodiment, the promoter comprises the at least 50 bp proximal to the transcription start site, for example, at least 25 bp. The promoter does not comprise exon and/or intron regions or 5′ untranslated regions. The promoter may for example be heterologous or homologous to the respective cell. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host cells (e.g. viruses). If a promoter is an inducible promoter, then the rate of transcription increases in response to an inducing agent. The term “constitutive” when made in reference to a promoter or the expression derived from a promoter means that the promoter is capable of directing transcription of an operably linked nucleic acid molecule in the absence of a stimulus (e.g., heat shock, chemicals, light, etc.) in cells throughout substantially the entire lifespan of said cell.

Promoter specificity: The term “specificity” when referring to a promoter means the pattern of expression conferred by the respective promoter. The specificity describes the developmental status of a cell, in which the promoter is conferring expression of the nucleic acid molecule under the control of the respective promoter. Specificity of a promoter may also comprise the environmental conditions, under which the promoter may be activated or down-regulated such as induction or repression by biological or environmental stresses such as cold, drought or infection.

Purified: As used herein, the term “purified” refers to molecules, either nucleic or amino acid sequences that are removed from their natural environment, isolated or separated.

“Substantially purified” molecules are at least 60% free, preferably at least 75% free, and more preferably at least 90% free from other components with which they are naturally associated. A purified nucleic acid sequence may be an isolated nucleic acid sequence.

Recombinant: The term “recombinant” with respect to nucleic acid molecules refers to nucleic acid molecules produced by recombinant DNA techniques. Recombinant nucleic acid molecules may also comprise molecules, which as such does not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

Sense: The term “sense” is understood to mean a nucleic acid molecule having a sequence which is complementary or identical to a target sequence, for example a sequence which binds to a protein transcription factor and which is involved in the expression of a given gene.

According to a preferred embodiment, the nucleic acid molecule comprises a gene of interest and elements allowing the expression of the said gene of interest.

Significant increase or decrease: An increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Small nucleic acid molecules: “small nucleic acid molecules” are understood as molecules consisting of nucleic acids or derivatives thereof such as RNA or DNA. They may be double-stranded or single-stranded and are between about 15 and about 30 bp, for example between 15 and 30 bp, more preferred between about 19 and about 26 bp, for example between 19 and 26 bp, even more preferred between about 20 and about 25 bp for example between 20 and 25 bp. In an especially preferred embodiment, the oligonucleotides are between about 21 and about 24 bp, for example between 21 and 24 bp. In a most preferred embodiment, the small nucleic acid molecules are about 21 bp and about 24 bp, for example 21 bp and 24 bp.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the latter being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

“Target region” as used herein means the region close to, for example 10 bases, 20 bases, 30 bases, 40 bases, 50 bases, 60 bases, 70 bases, 80 bases, 90 bases, 100 bases, 125 bases, 150 bases, 200 bases or 500 bases or more away from the target site, or including the target site in which the sequence of the donor DNA molecule is introduced into the genome of a cell.

“Target site” as used herein means the position in the genome at which a double strand break or one or a pair of single strand breaks (nicks) are induced using recombinant technologies such as Zn-finger, TALEN, restriction enzymes, homing endonucleases, RNA-guided nucleases, RNA-guided nickases such as CRISPR/Cas nucleases or nickases and the like.

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with a recombinant DNA molecule that preferably comprises a suitable promoter operatively linked to a DNA sequence of interest.

Vector. As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences recognized by any RNA polymerase, including mitochondrial RNA polymerase, RNA pol l, RNA pol II, and RNA pol Ill. These vectors can be used to transcribe the desired RNA molecule in the cell according to this invention.

Wild-type: The term “wild-type”, “natural” or “natural origin” means with respect to an organism, polypeptide, or nucleic acid sequence, that said organism is naturally occurring or available in at least one naturally occurring organism which is not changed, mutated, or otherwise manipulated by man.

### EXAMPLES

### 1) General

General reagents for microbial growth were obtained from Cari Roth GmbH. Catalase (as an example for an antioxidant agent), coniferyl alcohol, eugenol and other chemicals were purchased from Sigma Aldrich. HPLC-grade solvents were obtained from Honeywell or Chempur. For plasmid isolation, kits from New England Biolab Inc. or Qiagen GmbH were used. Other reagents for molecular biology and protein analysis were purchased from Thermofisher Scientific or GenScript. Synthetic genes were obtained from Biocat GmbH. DNA sequencing was performed at Microsynth AG. Protein quantification was performed with Bradford method (Bio-Rad Protein Assay Dye Reagent Concentrate).

### 2) Expression of Alkene Cleavage Oxygenase Variants and Preparation of Cell Free Extract

The codon optimised sequence of each CO was obtained in pET-51b(+) vector, with a Strep-Tag-II fused at the N-term of the amino acid sequence. Competent cells of E. coli BL21 (DE3) were transformed—via heat shock—with the pET-51b(+)_CO plasmid and finally, colonies were selected on LB-ampicillin (100 mg/L) agar plates (protocol from New England Biolabs Inc.) The day later one single colony was resuspended in LB medium (5 mL) supplemented with ampicillin (100 mg/L of culture) followed by overnight growth at 37° C. or 24 hours at 30° C. Then, 1% v/v of the pre-culture was transferred to a baffled flask containing terrific broth medium (TB) supplemented with ampicillin (100 mg/L), and incubated at 37° C. and 150 rpm. At OD₆₀₀˜0.5, iron salts were added to the culture to a final concentration of 100 mg/L each [Iron(II)sulphate heptahydrate, ammonium iron (II) sulphate hexahydrate and ammonium iron(Ill) citrate], then the cells were cooled at 20° C. for 15-20 min. Protein expression was induced with IPTG (0.5 mM) at OD₆₀₀˜0.8, and the culture was incubated for ˜20 hours at 20° C. and 100 rpm. At the end of the cultivation, the cells were harvested by centrifugation (R10A3 rotor, 4500 rpm, 4° C., 15 min), washed once with Glycine-NaOH buffer, (10 mM, pH 9.0) and stored at −20° C. Next, the cell pellet was resuspended in lysis buffer (50 mM Glycine-NaOH, 1 mM FeSO₄*H₂O, 1 mM ascorbate, pH 9.0; ˜4 mL of buffer per g of wet cell pellet) and disrupted by sonication (3 cycles with short ice incubation in between: 30% amplitude; 1 sec on; 2 sec off; 2 mins total pulse on; 6 min total time). Cell free extract was separated from the insoluble fraction by centrifugation (R20A2 rotor, 15000 rpm, 30 min, 4° C.) and freshly used for biotransformation experiments or freeze-dried and stored at −20° C. An example of the results is reported in Table 1.

TABLE 1

| Enzyme | EUGO | | IECO | | CO-03 | | | |
|---|---|---|---|---|---|---|---|---|
| Culture | 0.08 | L | 0.05 | L | 0.05 | L | 0.15 | L |
| Wet Cell Weight | 1.65 | g | 1.11 | g | 1.77 | g | 6.45 | g |
| Lysis Buffer | 11 | mL | 10 | mL | 13 | mL | 30 | mL |
| Freeze-dried powder | 210 | mg | 230 | mg | 220 | mg | 890 | mg |

| Buffer Content (w/w) | 6% | 22% | | 25% | | 14% | |
|---|---|---|---|---|---|---|---|
| Specific Activity | n.d. | 2.8 | mU/mg | 7-10 | mU/mg | 7-10 | mU/mg |
| (mU/mg of FD-CFE^{a}) | | | | | | | |

^{a}mg of FD-CFE refers to the effective freeze-dried powder, which also contains buffer salts.

### 3) Expression of EUGO and Preparation of Cell Free Extract

The codon optimised sequence of EUGO was obtained in pBAD vector, with a 6×His-Tag fused at the C-term of the amino acid sequence. Competent cells of E. coli NEB 10 Beta were transformed with pBAD_EUGO plasmid and colonies were selected on LB-ampicillin agar plates (Protocol from New England Biolabs Inc.). EUGO was recombinantly expressed according to a previously described protocol.^{[1]} In summary, a preculture was set up in LB-ampicillin (100 mg/mL) by resuspending single colony of E. coli NEB 10 Beta harbouring pBAD_EUGO plasmid. After overnight incubation at 37° C., 1% v/v of preculture was transferred to fresh TB medium supplemented with ampicillin (100 mg/L). Cells were grown at 37° C. and 120 rpm and induced with 0.02% arabinose at OD₆₀₀˜0.6. The protein expression was conducted for c.a. 20 hours at 30° C. Cell were harvested by centrifugation (R10A3 rotor, 4500 rpm, 4° C., 15 min), washed once with Tris-HCl (10 mM, pH 8.0) and stored at −20° C. For the preparation of the cell free extract, the cell pellet was resuspended in Tris-HCl (50 mM, pH 8.0; c.a. 4 mL per g or wet cell weight). Finally, the cells were disrupted by sonication (3 cycles with short ice incubation in between: 30% amplitude; 1 sec on; 2 sec off; 2 mins total pulse on; 6 mins total time). Cell free extract was separated from the insoluble fraction by centrifugation (R20A2 rotor, 15000 rpm, 30 min, 4° C.), freeze-dried and stored at −20° C. An example of the results is reported in Table 1 above.

### 4) Enzymatic Assay

Stock solution 200× of coniferyl alcohol (90 mg) was prepared in ethanol (5 mL) and stored at −20° C. From this, a new stock solution 20× of coniferyl alcohol (10 mM) was freshly prepared by 1:10 dilution in Glycine-NaOH (50 mM, pH 9.0). A cuvette containing 0.750 mL Glycine-NaOH (50 mM, pH 9.0) and 0.05 mL of the substrate 20× stock was pre-warmed at 25° C. for 1.5 mins. Then, the reaction was initiated by the addition of 0.2 mL of the enzyme sample. The cleaving 20 activity was measured spectrophotometrically following the production of vanillin at 349 nm (ε₃₄₉=26.7 mM⁻¹cm⁻¹ experimentally determined). One unit of activity (U) is defined as the amount of enzyme required to produce 1 μmol of vanillin per min. Agilent Cary 60 UV-Vis Spectrophotometer was used for all the measurements.

### 5) Biotransformation of Isoeugenol to Vanillin

### pH Screening

Stock solution of rac-isoeugenol (100 mM) was prepared in DMSO and stored at 4° C. Each reaction mixtures (1 mL) were set up as follows: rac-isoeugenol (50 μL, final concentration 5 mM) was diluted in various buffers (100 mM, pH 7.0-10.5) in 4 mL glass vials, together with FeSO₄ (0.5 mM) and DMSO (150 μL; final concentration including the part from substrate stock: 20% v/v). The reaction was initiated by the addition of IECO (cell free extract 1 mg/mL). Wild type E. coli BL21 (DE3) was used as negative control. The reaction was incubated for 24 hours at 30° C. and 120 rpm (rotary shaker). To improve the oxygenation, the vials were placed horizontally. The reaction was quenched with methanol (2 vol. equiv.), centrifuged at 20238 g for 5-10 mins and analysed by HPLC.

### Solvent Screening

Stock solutions of rac-isoeugenol (100 mM) were prepared in different solvents and stored in the dark at 4° C. Each reaction mixtures (1 mL) were set up as follows: ra-isoeugenol (50 μL, final concentration 5 mM) was diluted in Glycine-NaOH (100 mM, pH 9.5) in 4 mL glass vials, together with FeSO₄ (0.5 mM) and the respective cosolvent (0, 50 or 150 μL; final content of cosolvent including the part from the substrate stock: 5, 10 and 20% v/v). The reaction was initiated by the addition of IECO (cell free extract 1 mg/mL). Wild type E. coli BL21 (DE3) was used as negative control. The reaction was incubated for 24 hours at 30° C. and 120 rpm (rotary shaker). To improve the oxygenation, the vials were placed horizontally. The reaction was quenched with methanol (2 vol. equiv.), centrifuged at 20238 g and analysed by HPLC.

### 6) Biotransformation of Coniferyl Alcohol to Vanillin Catalysed by IECO

180 mg of coniferyl alcohol were weighted and dissolved in 10 mL ethanol and stored at −20° C. (stock 100 mM). The reaction mixtures (0.5 mL) were set up in 1.5 mL glass vials as follows: fresh cell free extract containing IECO was diluted in Glycine-NaOH (50 mM, pH 9.0) together with sodium ascorbate (1 mM), FeSO4 (1 mM) and bovine liver catalase (Sigma C40, 0-10 mg/mL). The reaction was initiated by the addition of coniferyl alcohol stock (0.025 mL, final concentration 5 mM), then it was incubated for 24 hours at 30° C. and 120 rpm (rotary shaker). Note that the reaction contained 5% v/v ethanol deriving from the substrate stock. To improve the oxygenation, the vials were placed horizontally. At certain time points, reaction samples were taken and quenched with methanol (2 vol. equivalents). The mixture was centrifuged for 5-10 mins at 20238 g. Then supernatant was finally transferred in a HPLC vial for analysis.

### 7) Bioconversion of Eugenol to Vanillin: On-Pot Cascade (5 mM Scale)

Stock solutions of eugenol (100 mM) were prepared in ethanol or DMSO and stored at −20° C. Unless otherwise stated, the reaction mixtures (0.5 mL) were set up in 1.5 mL glass vials as follows: freeze-dried cell free extract of IECO/CO-XX (3 mg) and EUGO (0.5 mg) were dissolved in the reaction buffer (50 mM glycine-NaOH pH 9.0) containing sodium ascorbate (1 mM), FeSO₄ (1 mM) and bovine liver catalase (5 mg, Sigma Aldrich C40). The reaction was initiated by the addition of 0.025 mL eugenol stock (final concentration 5 mM), then incubated at 30° C. and 120 rpm overnight (rotary shaker). The final content of cosolvent was 5% v/v. To improve oxygenation, the vials were placed horizontally. At certain time points, samples (0.2 mL) were quenched with methanol (0.4 mL) and the mixture was centrifuged at 20238 g for 5-10 mins. Finally, the supernatant was analysed by HPLC.

### 7) CO-03 Activity in Presence of Eugenol

Stock solutions of coniferyl alcohol (250 mM), eugenol (0-2 M) were prepared in ethanol and stored at −20° C. Unless otherwise specified, the reaction mixtures (0.5 mL) were set up in 1.5 mL glass vials as follows: CO-03 (freeze-dried CFE 5-10 mg/mL) was dissolved in buffer (Glycine-NaOH 50 mM, pH 9.0 or Tris-HCl 50 mM, pH 8.0) with sodium ascorbate (1 mM), FeSO4 (1 mM), bovine liver catalase (Sigma C40, 10 mg/mL) and eugenol (0-100 mM). The final content of ethanol was 5-7% v/v. The reaction was initiated by the addition of coniferyl alcohol (5 mM), and then incubated for 4 hours at 30° C. and 120 rpm (rotary shaker). To improve the oxygenation, the vials were placed horizontally.

### 8) Biotransformation of Eugenol to Coniferyl Alcohol (50 mM Scale)

The biotransformation (total volume 3 mL) was performed in a 10 mL round bottom flask with a stir bar. Eugenol (0.023 mL, 25 mg, 0.15 mmol, 1.0 equiv., 50 mM final conc.) was dissolved in DMSO or ethanol (0.150 mL). Then, the solution was diluted with Tis-HCl buffer (50 mM, pH 8.0, 2.827 mL). To obtain a homogeneous dispersion of the eugenol droplets, the mixture was stirred for about 15-20 mins. Then bovine liver catalase (Sigma P #C40, 30 mg, 2300 kU) and EUGO (30 mg of freeze-dried cell free extract, Tris base salt content was neglected) were added consecutively (note: EUGO was added after the catalase was completely dissolved). The reaction was incubated at room temperature and gentle stirring (40 rpm) for 17-24 hours. At certain time points, samples (0.1 mL) were diluted with methanol (0.8 mL, 8 vol equiv.) for stopping the reaction. The samples were centrifuged at 20238×g for 10 mins and the supernatant was analyzed by HPLC. When the reaction was completed, the products were extracted with ethyl acetate (3×1.5 mL EtOAc, in 2 mL microcentrifuge tubes). The aqueous and organic phases were separated by centrifugation (20238×g for ˜3 mins). Then, the organic phase was dried with NaSO4 and concentrated under reduced pressure (40° C., 200 mbar), but not dried completely to avoid polymerization. The concentrate was directly purified by silica gel chromatography (10 mL column volume) using a mixture of 1:1 etylacetate:cyclohexane as eluent. Fractions containing coniferyl alcohol (according to TLC) were combined and concentrated under reduced pressure (40° C., 200 mbar). Final drying under high vacuum (1.5 mbar) and inert gas flow yielded the title compound as yellowish solid (24.2 mg, 0.134 mmol, 90% yield). Notes: The obtained product was stored at −20° C. under inert gas atmosphere. Evaporation of a product solution in chloroform induced polymerization, therefore, no crude NMR was taken before product purification.

### 9) HPLC Analysis

The analysis of the reaction was carried out using Agilent Technologies 1260 Infinity HPLC system, equipped with a DAD detector. Compounds were separated by reversed phase with Luna 5 μm C18(2) 100 Å column, (length 250 mm; internal diameter 4.6 mm; product 00G4252-E0, Phenomenex®). The mobile phase was composed of water (Eluent A) and acetonitrile (Eluent B) both supplemented with 0.1% trifluoracetic acid. Unless otherwise stated, the following method was set: flow 1 mL/min; injection 5 μL; 0 min 70% A+30% B; 2 min 70% A+30% B; 10 min 100% B; 15 min 100% B; 17 min 70% A+30% B; 22 min 70% A+30% B. Eugenol, coniferyl alcohol and vanillin were detected at 280 nm, whereas coniferyl aldehyde was followed at 340 nm.

### 10) Testing of Combination of EUGO with IECO

Eugenol oxidase from Rhodococcus jostii (Uniprot: Q0SBK1) is a robust flavo-protein already applied in biocatalytic processes for the production of vanillin from vanillyl alcohol.^{[3,30]} It also catalyses the oxidation of eugenol 1a to coniferyl alcohol 2a with high efficiency (reported k_{cat}/Kₘ 3100·10³ s⁻¹ M⁻¹, pH 7.5), and it is active in a wide pH window with a preference of slightly basic conditions. Such features elect EUGO the ideal biocatalyst for the step 1 of the cascade. Then, Vanillin 3a can be hypothetically produced from coniferyl alcohol 2a by the oxidative cleavage of the C═C bond (with glycolaldehyde as coproduct). Although this specific enzymatic reaction has not been reported in literature, bacterial non-heme iron oxygenases have been found to catalyse the cleavage of isoeugenol 2b to vanillin 3a. Because of the similarity between the two substrates coniferyl alcohol and isoeugenol, it was tested whether such enzymes could accept coniferyl alcohol too. Three characterised isoeugenol cleavage oxygenases from Pseudomonas sp. (GenBank: AXB59146), Pseudomonas putida (GenBank: BAF62888) and Pseudomonas nitroreducens (GenBank: ACP17973) respectively, were selected. Among these, the latter could be recovered in a soluble form after recombinant expression in E. coli. A short characterisation showed that this variant from Pseudomonas nitroreducens (ECO) is active toward isoeugenol with an optimum at pH 9.0 and it tolerates a panel of cosolvents, with a preference for ethanol and DMSO. When IECO activity toward coniferyl alcohol was examined, 17% conversion to 3a was initially obtained, but the results were depending on the preparation used and a fraction of 2a was also transformed by endogenous enzymes from E. coli (Table 2).

TABLE 2
Enzymatic cleavage of coniferyl alcohol 2a with IECO after 24 hours incubation
(non-optimised reaction conditions). The biotransformation (1 mL) was carried
out in 4 mL glass vials: IECO (fresh or stored CFE 1,2 or 3 mg/mL) was diluted in
Glycine-NaOH (100 mM, pH 9.0) together with FeSO₄ (1 mM). The reaction was
initiated by the addition of coniferyl alcohol (5 mM). The mixture contained
5% v/v ethanol, deriving from the substrate stock (100 mM in ethanol 100%
v/v). The reaction was incubated at 30° C. and 120 rpm (rotary shaker).
To improve oxygenation, the vials were place horizontally.

| | CFE | CFE | Consumption | Conversion of 2a | Conversion of 2a |
|---|---|---|---|---|---|
| | Preparation^{a} | mg/mL | of 2a | to 3a (%) | to 5a (%) |
| IECO | Fresh | 1 | 24% | 9% | n.d. |
| | Fresh | 2 | 45% | 17% | n.d. |
| | Stored at −20° | 2 | 12% | 4% | 1% |
| | Stored at −20° C. | 3 | 18% | 5% | 13% |
| | Fresh | 1 | 31% | <1% | n.d. |
| Neg. Ctrl | Fresh | 2 | 49% | <1% | n.d. |
| | Stored at −20° | 2 | 25% | <1% | 21% |
| | Stored at −20° C. | 3 | 37% | <1% | 31% |

n.d. not determined.
Conversion = % of each product from 5 mM starting material
^{a}Storage buffer: Potassium phosphate (40 mM, pH 7.5), FeSO₄ (1 mM), sodium ascorbate (1 mM

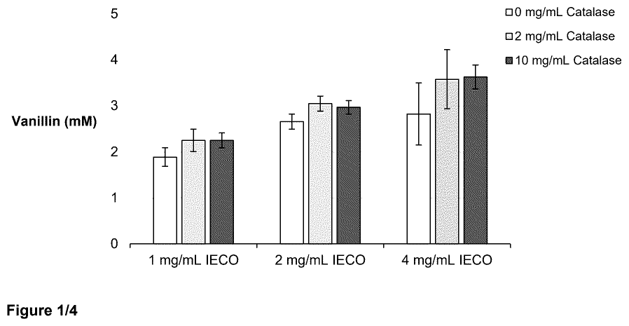

The results reported in Table 2 suggested that storage was critical for the activity of the enzyme; we then established that fresh cell free extract or storing the same in a freeze-dried form were the best options (data not shown). Pursing optimisation studies, we achieved 59±3% conversion with the same amount of catalyst as in the initial experiments (FIG. 1, IECO 2 mg CFE/mL). Overall, best conversion was obtained in the presence of catalase and increased amount of IECO (4 mg CFE/mL), affording 3.6±0.6 mM vanillin (equal to 73±5% conversion); see Table 3.

TABLE 3
Oxidative cleavage of coniferyl alcohol 2a to vanillin 3a at
varied amount of IECO and catalase after 24 hours. Each reaction
(0.5 mL) was set up in 1.5 mL glass vials with the following conditions:
Glycine-NaOH buffer (50 mM, pH 9.0), FeSO₄ (1 mM), sodium ascorbate
(1 mM), ethanol 5 vol %, IECO (fresh CFE 1-4 mg/mL), bovine
liver catalase (Sigma C40; 0-10 mg/mL = 0 to ≥100 kU/mL)
and coniferyl alcohol (5 mM). Each reaction was incubated at
30° C. and 120 rpm (rotary shaker). To improve oxygenation,
the vials were placed horizontally. The error was calculated
from the standard deviation of three independent experiments.
Conversion = % of 3a from 5 mM starting material

| IECO | | Reaction composition | | | Conversion |
|---|---|---|---|---|---|
| (CFE | Catalase | at 24 hours (%) | | | of 2a |
| mg/mL) | (mg/mL) | 2a | 3a | 5a | to 3a (%) |
| 1 | 0 | 46 ± 3 | 51 ± 3 | 3 ± 1 | 38 ± 4 |
| | 2 | 41 ± 6 | 58 ± 6 | 1 ± 0.1 | 44 ± 5 |
| | 10 | 41 ± 3 | 58 ± 1 | 1 ± 0.1 | 45 ± 3 |
| 2 | 0 | 25 ± 4 | 71 ± 4 | 5 ± 1 | 53 ± 3 |
| | 2 | 27 ± 3 | 72 ± 3 | 1 ± 0.3 | 58 ± 3 |
| | 10 | 22 ± 4 | 77 ± 4 | 1 ± 0.1 | 59 ± 3 |
| 4 | 0 | 9.1 ± 3 | 82 ± 5 | 9 ± 4 | 56 ± 10 |
| | 2 | 11 ± 3 | 84 ± 5 | 5 ± 4 | 62 ± 10 |
| | 10 | 13 ± 1 | 86 ± 13 | 1 ± 0.1 | 73 ± 5 |

### 11) One-Pot Concurrent Cascade

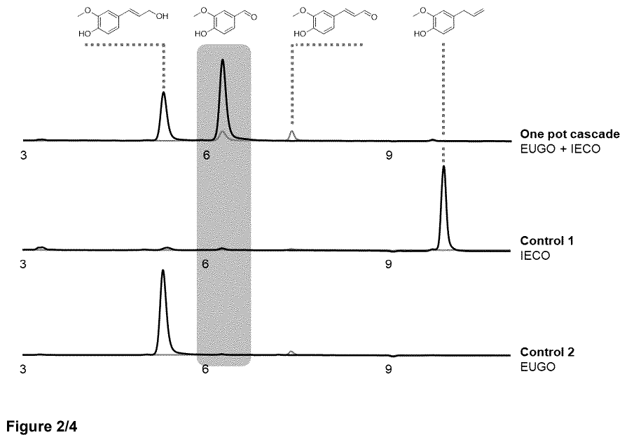

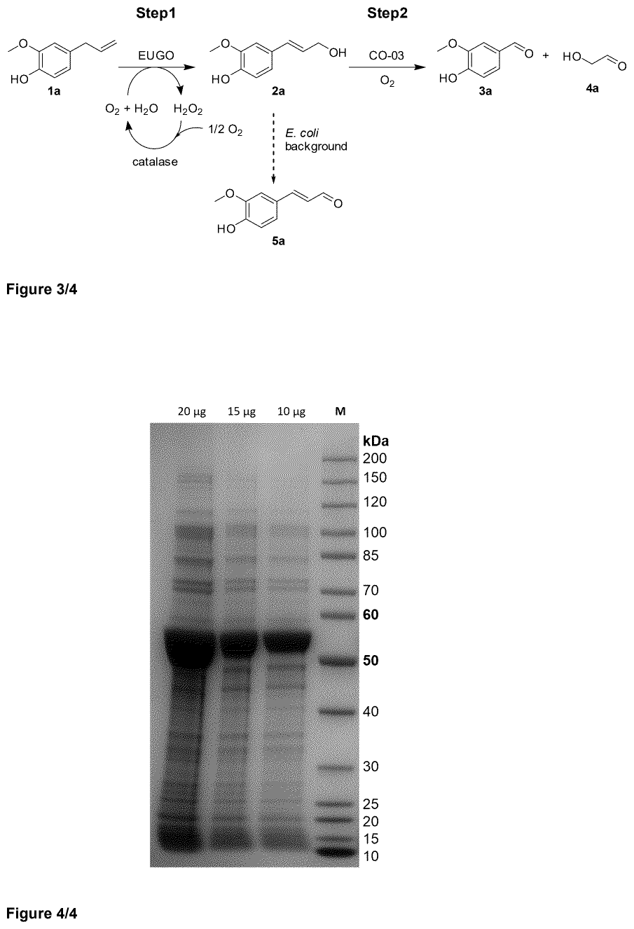

We then investigated the viability of our envisioned cascade reported in scheme 1c. We combined the two selected enzymes—EUGO and IECO—in a one pot concurrent cascade at a substrate 1a concentration of 5 mM. Remarkably, the tandem EUGO-IECO was indeed successful and 3a was formed (FIG. 2). After 24 hours incubation the first enzymatic step went to completion and 1a was not detectable anymore, which was also confirmed by the reaction control containing only EUGO (FIG. 2). Time studies then showed that this oxidation step occurs within the first two hours. Yet the reaction mixture contained also the cascade intermediate coniferyl alcohol 2a and its oxidation product coniferyl aldehyde 5a (FIG. 2 and Table 4). Thus, the limiting step remained the oxidative cleavage. Repeating the cascade and varying the amount of biocatalyst as well as pH are shown in Tables 5 to 7)

TABLE 4
Bioconversion of eugenol 1a with the tandem IECO and EUGO.
Reaction conditions (0.5 mL in 1.5 mL glass vials: EUGO (fresh
CFE 0 or 1 mg/mL) and IECO (fresh CFE 0 or 4 mg/mL) were dissolved
in Tris-HCl buffer (50 mM, pH 8.0) or Glycine-NaOH buffer (50 mM,
pH 9.0), containing also FeSO₄ (1 mM), sodium ascorbate (1
mM) and 5% v/v ethanol or DMSO. The reaction was initiated
by the addition of eugenol (0.025 mL, final concentration
5 mM) and incubated at 30° C. and 120 rpm. To improve
oxygenation, the vials were placed horizontally. Conversion =
% of 3a from 5 mM starting material

| | | | | | | | Conver- |
|---|---|---|---|---|---|---|---|
| EUGO | IECO | | Reaction composition | | | | sion of |
| (CFE | (CFE | Co- | at 24 hours (%) | | | | 1a to 3a |
| mg/mL) | mg/mL) | solvent | 1a | 2a | 3a | 5a | (%) |
| 1 | 4 | EtOH | 4 | 5 | 86 | 5 | 48 |
| 0 | 4 | | 97 | 2≤ | 1≤ | 1≤ | 1≤ |
| 1 | 0 | | 1≤ | 97 | 1≤ | 2 | 1≤ |
| 1 | 4 | DMSO | 3 | 49 | 44 | 4 | 42 |
| 0 | 4 | | 97 | 2≤ | 1≤ | 1≤ | 1≤ |
| 1 | 0 | | 1≤ | 96 | 1≤ | 2 | 1≤ |

TABLE 5
Bioconversion of eugenol 1a at varied amount of IECO and EUGO.
Reaction conditions (0.5 mL in 1.5 mL glass vials: EUGO (freeze-dried
CFE 1-5 mg/mL) and IECO (freeze-dried CFE 4-20 mg/mL) were
dissolved in Tris-HCl buffer (50 mM, pH 8.0) or Glycine-NaOH buffer
(50 mM, pH 9.0), containing also FeSO₄ (1 mM), sodium ascorbate
(1 mM) and bovine liver catalase (Sigma C40, 10 mg/mL). The mixture
contained 5% v/v ethanol deriving from the substrate stock. The reaction
was initiated by the addition of eugenol (0.025 mL, final concentration
5 mM) and incubated at 30° C. and 120 rpm. To improve oxygenation,
the vials were placed horizontally. Conversion = % of 3a from 5 mM
starting material

| EUGO | IECO | | Reaction composition | | | | Conversion |
|---|---|---|---|---|---|---|---|
| (CFE | (CFE | | at 24 hours (%) | | | | of 1a to 3a |
| mg/mL) | mg/mL) | pH | 1a | 2a | 3a | 5a | (%) |
| 1 | 4 | 8.0 | 1 | 5 | 45 | 1≤ | 40 |
| 2 | | 9.0 | 2 | 3 | 53 | 1≤ | 45 |
| | 4 | 8.0 | 2 | 4 | 48 | 2 | 40 |
| | | 9.0 | 1 | 4 | 52 | 2 | 44 |
| 1 | 6 | 8.0 | 2 | 4 | 49 | 1 | 44 |
| | | 9.0 | 3 | 3 | 59 | 1 | 50 |
| 2 | 6 | 8.0 | 3 | 5 | 44 | 3 | 41 |
| | | 9.0 | 3 | 4 | 50 | 2 | 49 |
| 5 | 20 | 8.0 | 1≤ | 27 | 9 | 6 | 8 |
| | | 9.0 | 1≤ | 18 | 15 | 5 | 13 |

TABLE 6
Replicates of the concurrent cascade using IECO or CO-03.
Each reaction was carried out as described in table 1. The
error was obtained from the standard deviation of four experiments.
Conversion = % of 3a from 5 mM starting material.

| | | | | | | Conversion |
|---|---|---|---|---|---|---|
| Cleavage | | Reaction composition at 24 hours (%) | | | | of 1a to 3a |
| Oxygenase | pH | 1a | 2a | 3a | 5a | (%) |
| IECO | 8.0 | 1≤ | 51 ± 2 | 34 ± 2 | 15 ± 3 | 31 ± 2 |
| | 9.0 | 1≤ | 22 ± 2 | 59 ± 1 | 18 ± 1 | 52 ± 3 |
| CO-03 | 8.0 | 1≤ | 14 ± 3 | 80 ± 2 | 9 ± 1 | 70 ± 3 |
| | 9.0 | 1≤ | 7 ± 2 | 83 ± 3 | 10 ± 1 | 70 ± 3 |

TABLE 7
Replicates of the biotransformation of eugenol 1a to coniferyl
alcohol 2a (50 mM scale). The reaction was carried out
in a round bottom flask with a total volume of 3 mL. Reaction
conditions: Tris-HCl (50 mM, pH 8.0), EUGO (FD-CFE 10 mg/mL),
bovine liver catalase (Sigma C40, 10 mg/mL), eugenol (50
mM), 5% v/v DMSO. The reaction was gentle stirred (30 rpm)
for 17-24 hours at room temperature.

| | Time | Reaction composition (%) | | | 2a |
|---|---|---|---|---|---|
| | (hours) | 1a | 2a | 5a | (mM) |
| Exp. 1 | 24 | 0 | 95 | 5 | 50 |
| Exp. 2 | 17 | 1 | 95 | 4 | 48 |
| Exp. 3 | 17 | 21 | 76 | 3 | 41 |

### 12) Screening for Further Isoeugenol Cleavage Oxygenases

Searching for alternative novel alkene cleavage oxygenases (COs) catalysing the formation of vanillin from coniferyl alcohol was performed. A set of 27 sequences and 10 COs from different branches of the phylogenetic tree were selected, named CO-02-11. Two additional enzymes were included, which are located in different clusters of the phylogenetic tree: Lignostillbene dioxygenase from Pseudomonas brassicaceraum (PbLSD, PDB: 5V2D) and aromatic dioxygenase (Ado, CO-01) from Thermothelomyces thermophilus (GenBank ID: XP_003665585). The proteins were recombinantly expressed in E. coli BL21(DE3), following the same protocol developed for IECO. Most of the candidates were obtained in soluble form and remarkably, CO-03, CO-4, CO-6 and CO-10 showed higher expression levels than IECO. Putative COs were screened by spectrophotometric assay, monitoring the formation of 3a upon 2a cleavage. From a set of twelve COs, four positive hits, namely CO-01, CO-03, CO-06 and CO-07 were found. Positive variants were tested in the one pot cascade and compared them with IECO In agreement with the spectrophotometric screening, all four COs converted eugenol 1a to vanillin 3a (Table 8). The first step of the cascade is efficiently performed by EUGO, and the substrate is completely consumed. Based on the CO (alkene cleavage oxigenase) used as well as the reaction conditions, the amount of products 3a, 5a, as well as the cascade intermediate 2a significantly varied (Table 8 and Table 6). The best cleaving enzyme was CO-03 with comparable amount of 3a at pH 8.0 and pH 9.0 (78-81%), whereas the remaining 2 and its oxidation product 5a were ˜20% of the reaction mixture. Overall, 70±3% conversion of 1a to vanillin 3a was achieved.

TABLE 8a
Bioconversion of eugenol 1a to vanillin 3a in a concurrent
2-step cascade with various alkene cleavage oxygenases.^{c}

| | | Reaction composition after 24 hours | | | |
|---|---|---|---|---|---|
| CleavingOxygenase | pH | 1a (%) | 2a (%) | 3a (%) | 5a (%) |
| Blank^{a} | 9.0 | 51 | 96 | ≤1 | 2 |
| Neg. Ctrl^{b} | 8.0 | ≤1 | 81 | 1≤ | 19 |
| | 9.0 | ≤1 | 69 | 1≤ | 30 |
| IECO | 8.0 | 51 | 52 | 36 | 12 |
| | 9.0 | ≤1 | 24 | 59 | 18 |
| CO-01 (Ado) | 8.0 | ≤1 | 74 | 9 | 17 |
| | 9.0 | 51 | 63 | 13 | 24 |
| CO-03 | 8.0 | ≤1 | 14 | 78 | 8 |
| | 9.0 | ≤1 | 8 | 81 | 11 |
| CO-06 | 8.0 | 51 | 79 | 5 | 16 |
| | 9.0 | ≤1 | 71 | 6 | 23 |
| CO-07 | 8.0 | 51 | 61 | 21 | 18 |
| | 9.0 | 51 | 50 | 27 | 23 |

^{a}Blank: the reaction did not contained any CO, but only EUGO and catalase.
^{b}Neg. Ctrl: the reaction contained the inactive CO-08 variant, EUGO and catalase.
^{c}Reaction conditions: EUGO (freeze-dried CFE 1 mg/mL), CO-XX (freeze-dried CFE 1 mg/mL), bovine liver catalase (10 mg/mL), FeSO4 (1 mM), sodium ascorbate (1 mM), eugenol (5 mM), 5% v/v ethanol in Tris-HCl pH 8.0 or glycine-NaOH pH 9.0. Incubation was carried out at 30° C. and 120 rpm (rotary shaker) for 24 hours. To improve oxygenation, the vials were placed horizontally. The experiment was performed in duplicate.

TABLE 8b
Bioconversion of eugenol 1a to vanillin 3a in a concurrent
2-step cascade with various alkene cleavage oxygenases

| | | | Product distribution after 24 hours^{b} | | | |
|---|---|---|---|---|---|---|
| | | | 1a | 2a | 3a | 5a |
| Cleaving Oxygenase | | pH | (%) | (%) | (%) | (%) |
| Controls | Neg. Ctrl^{c} | 8.0 | n.d. | 73.1 | 0.0 | 26.9 |
| | | 9.0 | n.d. | 67.9 | 0.0 | 32.1 |
| | IECO^{d} (Cluster 30) | 8.0 | n.d. | 67.8 | 17.6 | 14.6 |
| | | 9.0 | n.d. | 49.4 | 30.7 | 19.9 |
| | CO-03 (Cluster 30) | 8.0 | n.d. | 19.6 | 72.0 | 8.4 |
| | | 9.0 | n.d. | 11.9 | 74.9 | 13.2 |
| Cluster 30 | CO-13 | 8.0 | n.d. | 56.4 | 26.4 | 17.2 |
| | | 9.0 | n.d. | 45.3 | 32.4 | 22.3 |
| | CO-14 | 8.0 | n.d. | 78.9 | 5.1 | 16.0 |
| | | 9.0 | n.d. | 73.9 | 4.0 | 22.1 |
| | CO-15 | 8.0 | n.d. | 68.3 | 13.5 | 18.2 |
| | | 9.0 | n.d. | 61.2 | 16.5 | 22.2 |
| Cluster 19 | CO-16 | 8.0 | n.d. | 76.0 | 4.3 | 19.7 |
| | | 9.0 | n.d. | 71.0 | 5.5 | 23.5 |
| | CO-17 | 8.0 | n.d. | 73.1 | 7.8 | 19.1 |
| | | 9.0 | n.d. | 65.8 | 11.8 | 22.5 |
| Cluster 26 | CO-20 | 8.0 | n.d. | 59.4 | 23.6 | 17.0 |
| | | 9.0 | n.d. | 57.8 | 22.0 | 20.2 |
| | CO-22 | 8.0 | n.d. | 69.4 | 15.0 | 15.6 |
| | | 9.0 | n.d. | 63.2 | 16.8 | 20.0 |
| | CO-23 | 8.0 | n.d. | 69.0 | 13.7 | 17.3 |
| | | 9.0 | n.d. | 67.0 | 13.6 | 19.4 |
| | CO-24 | 8.0 | n.d. | 78.5 | 6.1 | 15.4 |
| | | 9.0 | n.d. | 73.9 | 5.6 | 20.5 |

^{a} Reaction conditions: EUGO (freeze-dried CFE 1 mg/mL), CO-0X (freeze-dried CFE 1 mg/mL), bovine liver catalase (10 mg/mL), FeSO₄ (1 mM), sodium ascorbate (1 mM), eugenol (5 mM), 5% v/v ethanol in tris-HCl pH 8.0 or glycine-NaOH pH 9.0. Incubation was carried out at 30° C. and 120 rpm (rotary shaker) for 24 hours. To improve oxygenation, the vials were placed horizontally. Each experiment was performed in duplicate. n.d. not detected.
^{b}Reaction composition: % of each compound identified by HPLC. Molar balance was not considered in the calculation.
^{c}Neg. Ctrl: the reaction contained E. coli BL21(DE3) lysate.
^{d}Batch with low specific activity.

In subsequent screenings for further alkene cleavage oxygenases, it was observed that the cleavage oxygenase from Novosphingobium aromaticivorans (NOV1, Uniprot: Q2GA76; herein also referred to as CO-25) showed a significant higher activity with coniferyl alcohol in comparison to a NOV1 enzyme in which the serine residue at position 282 had been replaced by phenylalanine (S283F; SEQ ID NO: 135). Therefore, 50 mL TB-Kan medium in a 250 mL shake flask were inoculated with an overnight preculture of E. coi BL21 (DE3) carrying a pET-28a plasmid encoding CO-25 in the expression cassette. At OD600 ˜0.8-1, the expression was induced with 0.1 mM IPTG and the culture incubated at 25° C. for 20 hours. 0.5 mM FeSO₄*7H₂O was added together with the inducer. Then, the cells were disrupted by sonication in Glycine-NaOH 50 mM pH 9.0 containing 1 mM FeSO₄*7H₂O and 1 mM sodium ascorbate. After centrifugation, the cell free extract was lyophilized and stored at −20° C. From 50 mL cell culture we obtained 235 mg of freeze-dried cell free extract. In this conditions, recombinant production of CO-25 was successful, proved by the sharp clear band visible on SDS-PAGE (FIG. 4).

### 13) Conversion of Eugenol to Vanillin by EUGO and CO-25

EUGO (0.5 mg CFE), CO-25 (3 mg CFE) and catalase (5 mg, Sigma product #C40) were dissolved in 50 mM Glycine-NaOH pH 9.0 or Tris-HCl pH 8.0 together with FeSO4 (1 mM) and sodium ascorbate (1 mM). Note that the buffer content of the freeze-dried biocatalyst prep was not considered for the assembling of the reaction. The reaction was started by the addition of 5 mM eugenol from a 20× stock in ethanol. The reactions—performed in glass vials—were then incubated at 30° C. and 120 rpm (25 mm shaker). Finally, samples of the reaction mixture were diluted with MeOH and analyzed by HPLC at 280 and 340 nm. Results at pH 8 and 9 were comparable, and as expected eugenol 1a was completely consumed. From 5 mM eugenol in 0.5 mL scale reaction, we obtained 83-88% conversion to vanillin 3a (Table 9).

TABLE 9
Bioconversion of eugenol 1a to vanillin
3a using EUGO and CO-25.

| | | | | | | | Intermediate and | | |
|---|---|---|---|---|---|---|---|---|---|
| | | | | | | | product distribution | | |
| | | | | | Recov- | | at 24 hours^{b} | | |
| | Sam- | 2a | 3a | 5a | ery | Conv.^{a} | 2a | 3a | 5a |
| pH | ple | [mM] | [mM] | [mM] | [%] | [%] | [%] | [%] | [%] |
| 8 | R1 | 4.18 | 4.18 | 0.03 | 101 | 83.5 | 16.4 | 83.1 | 0.5 |
| 8 | R2 | 4.39 | 4.39 | 0.03 | 106 | 87.7 | 17.1 | 82.4 | 0.5 |
| 9 | R1 | 4.31 | 4.31 | 0.05 | 100 | 86.2 | 12.8 | 86.3 | 0.9 |
| 9 | R2 | 4.21 | 4.21 | 0.04 | 96 | 84.2 | 11.6 | 87.5 | 0.9 |

^{a}Conv.: (3a mM)/5*100
^{b}Distribution at 24 hours: (2/3/5a mM)/(total recovered mM)*100

### 14) Conversion of Coniferylalcohol to Vanillin by CO-25

In each well of a deep-well-plate sealed with a gas permeable tape we mixed CO-25 (3 mg CFE) and catalase (4 mg) with coniferyl alcohol 2a (3.6 mg, 0.02 mL from 1M stock, final conc.: 50 mM) to a final volume of 0.4 mL. After one day incubation shaking at 290 rpm we obtained 40-46% conversion of coniferyl alcohol 2a to vanillin 3a (Table 10).

TABLE 10
Bioconversion of 50 mM coniferyl alcohol
2a to vanillin 3a using CO-25.

| | | | | | | | Intermediate and | | |
|---|---|---|---|---|---|---|---|---|---|
| | | | | | | | product distribution | | |
| | | | | | Recov- | | at 24 hours^{b} | | |
| | Sam- | 2a | 3a | 5a | ery | Conv.^{a} | 2a | 3a | 5a |
| pH | ple | [mM] | [mM] | [mM] | [%] | [%] | [%] | [%] | [%] |
| 8 | R1 | 21.48 | 23.01 | 0.52 | 90 | 46.0 | 47.7 | 51.1 | 1.1 |
| 9 | R1 | 22.87 | 19.92 | 0.81 | 87 | 39.8 | 52.5 | 45.7 | 1.9 |

^{a}Conv.: (3a mM)/5*100
^{b}Distribution at 24 hours: (2/3/5a mM)/(total recovered mM)*100

### 15) Substrate Loading and Scalability

Substrate loading is one of the challenges for the applicability of several biocatalytic processes.

Therefore, the cascade was tested by increasing the concentration of 1a to 50 mM. At this scale the reaction conditions significantly change; for example, 1a is insoluble and creates a separate phase, which might influence the biotransformation. Moreover, because of the structural similarity between 2a and 1a, the latter may be a competitive inhibitor of COs. Examining the activity of CO-03 incubated with different concentrations of 1a, we initially observed a drastic loss of activity at 50 mM 1a (Residual activity ≤10%). Note that by increasing the biocatalyst amount and modifying the reaction buffer such inhibition was mitigated and 49% of the cleaving activity could be retained. Scalability of step 1 (FIG. 1) was successfully proven. Thus, when EUGO (Cell free extract form) was incubated with 50 mM 1a, the substrate was consumed within 24 hours; HPLC analysis showed a main product peak corresponding to 2a, yet a fraction of 5a was also present. Overall, 95% of the desired product 2a and 5% of 5a, was obtained which was confirmed by a second replicate (Table 7).

### 16) Summary

The isoeugenol cleavage oxygenase from Pseudomonas nitroreducens (IECO; SEQ ID NO:24) has a very narrow substrate scope. When the enzymatic C═C cleavage of coniferyl alcohol was tested, initially 17% conversion to vanillin was obtained. The addition of catalase pushes IECO activity reaching up to 73% conversion for the transformation of coniferyl alcohol to vanillin. Furthermore, the sequence space of the phylogenetic cluster of IECO by screening putative sequences of the carotenoid oxygenase family was explored, and additional new enzymes which catalyse the formation of vanillin from coniferyl alcohol were found. This activity is not restricted to the cluster of IECO, but spread in the enzyme family. The tested active alkene cleavage oxygenases were successfully applied in the de novo 2-step cascade of the present invention for the synthesis of vanillin. Best results were obtained with IECO and CO-03 with 50% and 70% conversion, respectively at 5 mM substrate concentration.

1.-64. (canceled)

65. A process for producing vanillin, comprising the steps of
i. Providing an aqueous medium comprising a solvent, one or more eugenol oxidase, one or more alkene cleavage oxygenase and eugenol,
ii. Incubating the aqueous medium and
iii. Optionally isolating the vanillin, from the reaction mixture,
wherein the one or more eugenol oxidase is capable of catalysing the reaction from eugenol to coniferyl alcohol in an aqueous medium comprising water, eugenol oxidase and eugenol, and
wherein the one or more alkene cleavage oxygenase is capable of catalysing the reaction from coniferyl alcohol to vanillin in an aqueous medium comprising water, alkene cleavage oxygenase and coniferyl alcohol,
wherein after incubation at least 20% of the eugenol have been converted to vanillin.

66. The process of claim 65, wherein the eugenol oxidase is comprising a sequence selected from the group consisting of
a. The amino acid molecule of SEQ ID NO: 2,
b. An amino acid molecule having at least 50% identity to the amino acid molecule of SEQ ID NO: 2, or a functional fragment thereof,
c. An amino acid molecule encoded by a nucleic acid molecule of SEQ ID NO: 1 or a functional fragment thereof, and
d. An amino acid molecule encoded by a nucleic acid molecule having at least 50% identity to SEQ ID NO: 1, or a functional fragment thereof, and
wherein the amino acid molecule as defined in b, c, d is catalysing the reaction from eugenol to coniferyl alcohol in an aqueous medium.

67. The process of claim 65, wherein the alkene cleavage oxygenase is comprising a sequence selected from the group consisting of
e. The amino acid molecule of SEQ ID NO: 30, 24, 26, 38, 40, 64, 66, 74, 76, 82, 98, 120, 128, 130, 132, 134 or 135 and
f. An amino acid molecule having at least 50% identity to the amino acid molecule of SEQ ID NO: 30, 24, 26, 38, 40, 64, 66, 74, 76, 82, 98, 120, 128, 130, 132, 134 or 135, or a functional fragment thereof, and
g. An amino acid molecule encoded by a nucleic acid molecule of SEQ ID NO: 29, 23, 25, 37, 39, 63, 65, 73, 75, 81, 97, 119, 127, 129, 131 or 133, or a functional fragment thereof, and
h. An amino acid molecule encoded by a nucleic acid molecule having at least 50% identity to SEQ ID NO: 29, 23, 25, 37, 39, 63, 65, 73, 75, 81, 97, 119, 127, 129, 131, or 133, or a functional fragment thereof, and
wherein the amino acid molecule as defined in f, g, h is catalysing the reaction from coniferyl alcohol or isoeugenol to vanillin in an aqueous medium.

68. A recombinant construct comprising a nucleic acid molecule encoding a eugenol oxidase and a nucleic acid molecule encoding a alkene cleavage oxygenase,
wherein the eugenol oxidase is comprising a sequence encoding an amino acid molecule selected from the group consisting of
a. The amino acid molecule of SEQ ID NO: 2 and
b. An amino acid molecule having at least 50% identity to the amino acid molecule of SEQ ID NO: 2 or a functional fragment thereof,
c. An amino acid molecule encoded by a nucleic acid molecule of SEQ ID NO: 1 or a functional fragment thereof, and
d. An amino acid molecule encoded by a nucleic acid molecule having at least 50% identity to SEQ ID NO: 1 or a functional fragment thereof, and
wherein the amino acid molecule as defined in b, c, d is catalysing the reaction from eugenol to coniferyl alcohol in an aqueous medium, and
wherein the alkene cleavage oxygenase is comprising a sequence encoding an amino acid molecule selected from the group consisting of
e. The amino acid molecule of SEQ ID NO: 30, 24, 26, 38, 40, 64, 66, 74, 76, 82, 98, 120, 128, 130, 132, 134 or 135, and
f. An amino acid molecule having at least 50% identity to the amino acid molecule of SEQ ID NO: 30, 24, 26, 38, 40, 64, 66, 74, 76, 82, 98, 120, 128, 130, 132, 134 or 135, or a functional fragment thereof, and
g. An amino acid molecule encoded by a nucleic acid molecule of SEQ ID NO: 29, 23, 25, 37, 39, 63, 65, 73, 75, 81, 97, 119, 127, 129, 131, or 133, or a functional fragment thereof, and
h. An amino acid molecule encoded by a nucleic acid molecule having at least 50% identity to SEQ ID NO: 29, 23, 25, 37, 39, 63, 65, 73, 75, 81, 97, 119, 127, 129, 131, or 133, or a functional fragment thereof, and
wherein the amino acid molecule as defined in f, g, h is catalysing the reaction from coniferyl alcohol to vanillin in an aqueous medium.

69. The recombinant construct of claim 68, wherein each the nucleic acid molecule encoding the eugenol oxidase and the nucleic acid molecule encoding the alkene cleavage oxygenase are functionally linked to a heterologous promoter.

70. A recombinant microorganism comprising the recombinant construct of claim 4 or a recombinant vector comprising the recombinant construct of claim 68.

71. A composition comprising water, one or more eugenol oxidase and one or more alkene cleavage oxygenase, eugenol,
wherein the eugenol oxidase is selected from the group consisting of
a. The amino acid molecule of SEQ ID NO: 2 and
b. An amino acid molecule having at least 50% identity to the amino acid molecule of SEQ ID NO: 2 or a functional fragment thereof, and
c. An amino acid molecule encoded by a nucleic acid molecule of SEQ ID NO: 1 or a functional fragment thereof, and
d. An amino acid molecule encoded by a nucleic acid molecule having at least 50% identity to SEQ ID NO: 1 or a functional fragment thereof, and
wherein the amino acid molecule as defined in b, c, d is catalysing the reaction from eugenol to coniferyl alcohol in an aqueous medium and
wherein the alkene cleavage oxygenase is selected from the group consisting of
e. The amino acid molecule of SEQ ID NO: 30, 24, 26, 38, 40, 64, 66, 74, 76, 82, 98, 120, 128, 130, 132, 134 or 135, and
f. An amino acid molecule having at least 50% identity to the amino acid molecule of SEQ ID NO: 30, 24, 26, 38, 40, 64, 66, 74, 76, 82, 98, 120, 128, 130, 132, 134 or 135, or a functional fragment thereof, and
g. An amino acid molecule encoded by a nucleic acid molecule of SEQ ID NO: 29, 23, 25, 37, 39, 63, 65, 73, 75, 81, 97, 119, 127, 129, 131, or 133, or a functional fragment thereof, and
h. An amino acid molecule encoded by a nucleic acid molecule having at least 50% identity to SEQ ID NO: 29, 23, 25, 37, 39, 63, 65, 73, 75, 81, 97, 119, 127, 129, 131, or 133, or a functional fragment thereof, and
wherein the amino acid molecule as defined in f, g, h is catalysing the reaction from coniferyl alcohol to vanillin in an aqueous medium.

72. A recombinant microorganism comprising an introduced, increased or enhanced activity and/or expression of one or more eugenol oxidase and one or more alkene cleavage oxygenase,
wherein the one or more eugenol oxidase is capable of catalysing the reaction from eugenol to coniferyl alcohol, and
wherein the one or more alkene cleavage oxygenase is capable of catalysing the reaction from coniferyl alcohol to vanillin, and
wherein the eugenol oxidase is comprising a sequence selected from the group consisting of
a. The amino acid molecule of SEQ ID NO: 2 and
b. An amino acid molecule having at least 50% identity to the amino acid molecule of SEQ ID NO: 2 or a functional fragment thereof, and
c. An amino acid molecule encoded by a nucleic acid molecule of SEQ ID NO: 1 or a functional fragment thereof, and
d. An amino acid molecule encoded by a nucleic acid molecule having at least 50% identity to SEQ ID NO: 1 or a functional fragment thereof, and
wherein the amino acid molecule as defined in b., c., d is catalysing the reaction from eugenol to coniferyl alcohol and
wherein the alkene cleavage oxygenase is comprising a sequence selected from the group consisting of
e. The amino acid molecule of SEQ ID NO: 30, 24, 26, 38, 40, 64, 66, 74, 76, 82, 98, 120, 128, 130, 132, 134 or 135, and
f. An amino acid molecule having at least 50% identity to the amino acid molecule of SEQ ID NO: 30, 24, 26, 38, 40, 64, 66, 74, 76, 82, 98, 120, 128, 130, 132, 134 or 135, or a functional fragment thereof, and
g. An amino acid molecule encoded by a nucleic acid molecule of SEQ ID NO: 29, 23, 25, 37, 39, 63, 65, 73, 75, 81, 97, 119, 127, 129, 131, or 133, or a functional fragment thereof, and
h. An amino acid molecule encoded by a nucleic acid molecule having at least 50% identity to SEQ ID NO: 29, 23, 25, 37, 39, 63, 65, 73, 75, 81, 97, 119, 127, 129, 131, or 133, or a functional fragment thereof, and
wherein the amino acid molecule as defined in f, g, h is catalysing the reaction from coniferyl alcohol to vanillin.

73. A method for fermentative production of vanillin, comprising the steps of
i. Providing a recombinant microorganism of claim 72,
ii. Culturing said microorganism in a medium comprising eugenol under conditions that allow for the production of said vanillin, and optionally isolating said vanillin, from the medium.

74. A composition comprising one or more recombinant microorganisms according to claim 72.

75. The composition of claim 74 further comprising eugenol, a medium.

76. The microorganism of claim 72, wherein the microorganism is selected from Rhodococcus rhodochrous, Aerococcus sp., Aspergillus sp., Bacillus pumilus, Bacillus subtilis, Bacteroides thetaiotaomicron, Clostridium algidicarnis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica.

77. A use of a recombinant microorganism according to claim 72 for the whole cell bio-conversion of eugenol to vanillin.

78. A process for whole cell bio-conversion of eugenol to vanillin comprising the steps of
I) growing the recombinant microorganism according to claim 72 in a fermenter comprising eugenol, a medium suitable for growing said recombinant microorganism and
II) recovering the vanillin thereof from the fermentation broth obtained in I).

79. A process for whole cell bio-conversion of eugenol to vanillin comprising the steps of
i) growing the recombinant microorganism according to claim 72 in a fermenter comprising a medium suitable for growing said recombinant microorganism, and
ii) recovering the recombinant microorganism from the fermenter, and
iii) performing a whole cell bio-conversion in an aqueous medium by supplementing eugenol, and
iv) recovering vanillin from the aqueous medium obtained in iii).


SEQUENCE LISTING (135 sequences)

SEQ ID NO: 1 - DNA, 1602 bp, synthetic construct
  source 1..1602: mol_type = other DNA
atgacccgtaccctgccgccgggcgtgagtgatgaacgctttgatgccgccctgcagcgttttcgtgatgttgtgggtgacaaatgggtgctgagtaccgccgatgaactggaagcatttcgtgatccgtatccggtgggcgcagcagaagcaaatctgccgagtgcagttgttagtccggaaagtaccgaacaggttcaggatattgttcgtattgccaatgaatatggtattccgctgagcccggttagtaccggtaaaaataatggctatggcggtgcagcaccgcgcctgagtggtagtgttattgttaaaaccggcgaacgtatgaatcgtattctggaagttaatgaaaagtacggttatgccctgctggaaccgggtgttacctattttgatctgtatgaatatctgcagagtcatgatagtggtctgatgctggattgtccggatctgggctggggtagcgttgtgggtaataccctggatcgcggtgtgggctataccccgtatggcgatcattttatgtggcagaccggtctggaagtggttctgccgcagggtgaagtgatgcgtaccggcatgggcgcactgccgggcagtgatgcatggcagctgtttccgtatggttttggtccgtttccggatggcatgtttacccagagtaatctgggtattgttaccaaaatgggtattgcactgatgcagcgcccgccggccagtcagagctttctgattacctttgataaagaagaagacctggaacagattgttgatattatgctgccgctgcgcattaatatggcaccgctgcagaatgttccggtgctgcgtaatatttttatggatgcagcagccgttagcaaacgtaccgaatggtttgatggtgacggcccgatgccggcagaagccattgaacgcatgaaaaaagatctggatctgggtttttggaatttttatggcaccctgtatggcccgccgccgctgattgaaatgtattatggcatgattaaggaggcctttggtaaaattccgggtgcacgtttctttacccatgaagaacgtgatgatcgcggtggtcatgttctgcaggatcgccataaaattaataatggtattccgagtctggatgaactgcagctgctggattgggttccgaatggtggccatattggttttagtccggtgagtgccccggatggtcgcgaagccatgaaacagtttgaaatggttcgcaatcgtgcaaatgaatataataaggattacgcagcccagtttattattggtctgcgtgaaatgcatcatgtttgtctgtttatctatgataccgccattccggaagcacgcgaagaaattctgcagatgaccaaagttctggtgcgcgaagccgcagaagccggttatggtgaatatcgcacccataatgccctgatggatgatgtgatggcaacctttaattggggcgatggtgcactgctgaaatttcatgaaaaaattaaggacgccctggatccgaatggtattattgccccgggcaaaagcggcatttggagtcagcgttttcgcggccagaatctgcatcatcatcatcaccattaataa

SEQ ID NO: 2 - protein, 526 aa, Rhodococcus Jostii
MTRTLPPGVSDERFDAALQRFRDVVGDKWVLSTADELEAFRDPYPVGAAEANLPSAVVSPESTEQVQDIVRIANEYGIPLSPVSTGKNNGYGGAAPRLSGSVIVKTGERMNRILEVNEKYGYALLEPGVTYFDLYEYLQSHDSGLMLDCPDLGWGSVVGNTLDRGVGYTPYGDHFMWQTGLEVVLPQGEVMRTGMGALPGSDAWQLFPYGFGPFPDGMFTQSNLGIVTKMGIALMQRPPASQSFLITFDKEEDLEQIVDIMLPLRINMAPLQNVPVLRNIFMDAAAVSKRTEWFDGDGPMPAEAIERMKKDLDLGFWNFYGTLYGPPPLIEMYYGMIKEAFGKIPGARFFTHEERDDRGGHVLQDRHKINNGIPSLDELQLLDWVPNGGHIGFSPVSAPDGREAMKQFEMVRNRANEYNKDYAAQFIIGLREMHHVCLFIYDTAIPEAREEILQMTKVLVREAAEAGYGEYRTHNALMDDVMATFNWGDGALLKFHEKIKDALDPNGIIAPGKSGIWSQRFRGQNL

SEQ ID NO: 3 - DNA, 1581 bp, Rhodococcus wratislaviensis
  source 1..1581: mol_type = genomic DNA
atgacccgtacccttccccctggcgtttccgacgagcgattcgacgcagcgctccagcgcttccgcgacgtcgtgggtgacaagtgggtcctgtcgaccgccgacgaactcgaagcgttccgcgacccgtacccggtcggcgctgccgaggccaatatgccgtcggccgtcgtctcaccggagtcgaccgagcaggtacaggacatcgtccggatcgccaacgagtacggcatccccctctccccggtctcgaccggaaagaacaacggttacggcggcgccgccccgaggctgtccggttcggtgatcgtcaagaccggcgagcggatgaacagaatcctcgaggtgaacgagaagtacggttacgcactgctcgaacccggggtcacctacttcgacctgtacgagtacctccagagccacgattccggcctgatgctcgactgtcccgatctgggatggggcagcgtcgtcggaaatgccctggaccgcggcgtcggatacaccccgtacggcgatcacttcatgtggcagacgggtctggaagtggttctgccgcagggcgatgtgatgcgcaccggcatgggcgcactccccggcagcgacgcgtggcaactgttcccctacgggttcggccccttcccggacggcatgttcacccagtccaatctcgggatcgtcaccaagatggggatcgccctgatgcagcggccgcccgcgtcgcagtcgttcctgatcacgttcgacaaggaggaggatctggagcagatcgtcgacatcatgcttccgctacgcatcaatatggcaccgctgcagaatgttccggtgctgcgcaacatcttcatggacgcggccgcggtgtcgaagcgcaccgagtggttcgacggcgaggggccgatgccggccgaggcggtcgagaggatgaagaaggatctcgatctcggattctggaacttctacggcaccctgtacggacctccgccgctgatcgagatgtactacgggatgatcaaggaggcgttcggcaagattcccggcgcccgtttcttcacacacgaggaacgccacgatcgcggtgggcatgtgttgcaggacaggcacaagatcaacaacggcatccccagcctggacgaactgcaactgctcgactgggtgcccaacggcgggcacatcgggttttccccggtgtcggcgccggacggccgcgaggcgatgaaacagttcgagatggtccgcgctcgcgccgacgaatacgccaaggactacgcggcgcaattcatcatcggactccgcgagatgcaccacgtgtgcctgttcatctacgacacggcgattcccgacgctcgtgaggaaatcctgcagatgacgaaggtcctcgtccgggaggccgccgaagccgggtacggcgaataccgcacccacaacgccctgatggacgacgtgatggcgacgttcgactggggtgacggtgcgctgctgaagttccacgagaagatcaaggacgccctcgaccccaacggcatcatcgcgcccggcaagtccggtgtgtggccgcaacggttccgtggccagaacctctga

SEQ ID NO: 4 - protein, 526 aa, Rhodococcus wratislaviensis
MTRTLPPGVSDERFDAALQRFRDVVGDKWVLSTADELEAFRDPYPVGAAEANMPSAVVSPESTEQVQDIVRIANEYGIPLSPVSTGKNNGYGGAAPRLSGSVIVKTGERMNRILEVNEKYGYALLEPGVTYFDLYEYLQSHDSGLMLDCPDLGWGSVVGNALDRGVGYTPYGDHFMWQTGLEVVLPQGDVMRTGMGALPGSDAWQLFPYGFGPFPDGMFTQSNLGIVTKMGIALMQRPPASQSFLITFDKEEDLEQIVDIMLPLRINMAPLQNVPVLRNIFMDAAAVSKRTEWFDGEGPMPAEAVERMKKDLDLGFWNFYGTLYGPPPLIEMYYGMIKEAFGKIPGARFFTHEERHDRGGHVLQDRHKINNGIPSLDELQLLDWVPNGGHIGFSPVSAPDGREAMKQFEMVRARADEYAKDYAAQFIIGLREMHHVCLFIYDTAIPDAREEILQMTKVLVREAAEAGYGEYRTHNALMDDVMATFDWGDGALLKFHEKIKDALDPNGIIAPGKSGVWPQRFRGQNL

SEQ ID NO: 5 - DNA, 1581 bp, Rhodococcus sp.
  source 1..1581: mol_type = genomic DNA
atgacgcgaacccttccccccggcgtttccgacgagcgattcgacgcggcgctccaacgcttccgcgacatcgtgggcgacaagtgggtcctgtcgaccgccgacgagctcgaagcgttccgcgacccgtacccggtcggcgccgccgaggccaacatgccgtcggccgtcgtctcaccggaatcgaccgagcaggtacaggacatcgtccggatcgccaacgaatacggcatccccctctccccggtctcgaccggaaagaacaacggctacggcggcgccactccgaggctgtcgggttcggtgatcgtcaagacgggcgagcggatgaaccggatcctcgaggtcaacgagaagtacggctacgcactgctcgaacccggggtcacctacttcgacctctacgactatctgcagagccacgattccggactgatgctcgactgccccgacctggggtggggcagcgtggtcggtaacactctggaccgcggcgtcgggtacaccccgtacggcgaccacttcatgtggcagacgggcctggaagtggtgctgccgcaaggtgatgtgatgcgcaccgggatgggcgcactacccggcagcgacgcgtggcagctgtttccctacgggttcggccccttcccggacggcatgttcacccagtcgaatctcgggatcgtcaccaagatggggatcgccctgatgcagcggccgcccgcgtcgcagtcgttcctgatcaccttcgacaaggaggaggatctggagcagatcgtcgacatcatgctgccgctgcgcatcaatatggctccgctgcagaatgttccggtgctgcgcaacatcttcatggacgcggccgcggtgtcgaagcgcaccgagtggttcgacggtgacgggccgatgccggccgaggcgatcgagaggatgaagaaagacctcgatctcggattctggaacttctacggcaccctgtacggacctccgccgctgatcgagatgtactacgggatgatcaaggaggcgttcggcaagattcccggtgcccgcttcttcacacacgaggaacgcgccgaccgcggtggacatgtgttgcaggacaggcacaagatcaacaacggcgtccccagcctggacgaactgcagctcctggactgggtacccaacggcgggcacatcggattctccccggtgtcggcgccggacggccgggaggcgatgaagcagttcgagatggtgcgcgctcgcgccgacgaatatgccaaggactacgcggcgcaattcatcatcggactccgcgagatgcaccacgtgtgcctgttcatctacgacacggcgatccccgacgctcgtgaggaaatcctgcagatgacgaaggtcctcgtccgcgaggccgccgacgcgggctacggcgagtaccgcacccacaacgccctgatggacgacgtgatggctacgttcgactggggcgacggtgcgctgctgaagttccacgagaagatcaaggacgccctcgatcccaacggcatcatcgcgcccggcaagtccggtgtgtggccgcaacggttccgtggccagaagctctga

SEQ ID NO: 6 - protein, 526 aa, Rhodococcus sp.
MTRTLPPGVSDERFDAALQRFRDIVGDKWVLSTADELEAFRDPYPVGAAEANMPSAVVSPESTEQVQDIVRIANEYGIPLSPVSTGKNNGYGGATPRLSGSVIVKTGERMNRILEVNEKYGYALLEPGVTYFDLYDYLQSHDSGLMLDCPDLGWGSVVGNTLDRGVGYTPYGDHFMWQTGLEVVLPQGDVMRTGMGALPGSDAWQLFPYGFGPFPDGMFTQSNLGIVTKMGIALMQRPPASQSFLITFDKEEDLEQIVDIMLPLRINMAPLQNVPVLRNIFMDAAAVSKRTEWFDGDGPMPAEAIERMKKDLDLGFWNFYGTLYGPPPLIEMYYGMIKEAFGKIPGARFFTHEERADRGGHVLQDRHKINNGVPSLDELQLLDWVPNGGHIGFSPVSAPDGREAMKQFEMVRARADEYAKDYAAQFIIGLREMHHVCLFIYDTAIPDAREEILQMTKVLVREAADAGYGEYRTHNALMDDVMATFDWGDGALLKFHEKIKDALDPNGIIAPGKSGVWPQRFRGQKL

SEQ ID NO: 7 - DNA, 1581 bp, Rhodococcus opacus
  source 1..1581: mol_type = genomic DNA
atgacgcgaacccttccccctggcgtttccgacgagcgattcgaggcggcgctccagcgcttccgcgacgtcgtgggtgacaagtgggtcctgtcgaccgcagacgaactcgaagcgttccgcgacccgtacccggtcggtgccgccgaggccaacatgccgtcggcggtcgtctcaccggagtcgaccgagcaggtccaggacatcgtccggatcgccaacgagtacggcatccccctctccccggtctcgaccggaaagaacaacggttacggcggcgccgccccgaggctgtcgggttcggtgatcgtcaagaccggcgagcggatgaacagaatcctcgaggtcaacgagaagtacggctacgcactgctcgaacccggggtcacctacttcgacctgtacgaatacctccagagccacgattccggcctgatgctcgactgtcccgacctgggatggggcagcgtggtggggaacaccctggaccgcggcgtcgggtacaccccgtacggcgatcacttcatgtggcagacgggactggaagtggttctgccgcagggtgatgtgatgcgcaccggcatgggcgcactgcccggcagcgacgcgtggcagttgttcccctacgggttcggtcccttcccggacggcatgttcacccagtcgaatctcgggatcgtcaccaagatggggatcgccctgatgcagcggccgcccgcgtcgcagtcgttcctgatcaccttcgacaaggaggaggatctcgagcaaatcgtcgacatcatgctgccgctgcgcatcaatatggctccgctgcagaatgttccggtgctgcgcaacatcttcatggacgcggccgcggtgtcgaagcgcaccgagtggttcgacggcgacgggccgatgccggccgaggcgatcgagaggatgaaaaaggatctcgatctcgggttctggaacttctacggcaccctgtacggacctccgccgctgatcgagatgtactacgggatgatcaaggaggcgttcggcaagattcccggtgcccgcttcttcacacacgaggaacgcgacgaccgcggtggacatgtgttgcaggacaggcacaagatcaacaacggcatccccagcctggacgaactgcaactcctggactgggtgcccaacggcgggcacatcggattctccccggtgtcggcgccggacggtcgcgaggcgatgaagcagttcgagatggtgcgcgctcgcgccgacgaatacgccaaggactacgccgcgcaattcatcatcgggctccgcgagatgcaccacgtgtgcctgttcatctacgacacggcgatccccgatgctcgtgaggaaatcctgcagatgacgaaggtcctcgtccgcgaggccgccgaagccggttacggcgaataccgcacccacaacgccctgatggacgacgtgatggcgacgttcgactggggcgacggtgcgctgctgaagttccacgagaagatcaaggacgccctcgaccccaacggcatcatcgcgcccggcaagtccggtgtgtggccgcagcggttccgtggccagaacctctga

SEQ ID NO: 8 - protein, 526 aa, Rhodococcus opacus
MTRTLPPGVSDERFEAALQRFRDVVGDKWVLSTADELEAFRDPYPVGAAEANMPSAVVSPESTEQVQDIVRIANEYGIPLSPVSTGKNNGYGGAAPRLSGSVIVKTGERMNRILEVNEKYGYALLEPGVTYFDLYEYLQSHDSGLMLDCPDLGWGSVVGNTLDRGVGYTPYGDHFMWQTGLEVVLPQGDVMRTGMGALPGSDAWQLFPYGFGPFPDGMFTQSNLGIVTKMGIALMQRPPASQSFLITFDKEEDLEQIVDIMLPLRINMAPLQNVPVLRNIFMDAAAVSKRTEWFDGDGPMPAEAIERMKKDLDLGFWNFYGTLYGPPPLIEMYYGMIKEAFGKIPGARFFTHEERDDRGGHVLQDRHKINNGIPSLDELQLLDWVPNGGHIGFSPVSAPDGREAMKQFEMVRARADEYAKDYAAQFIIGLREMHHVCLFIYDTAIPDAREEILQMTKVLVREAAEAGYGEYRTHNALMDDVMATFDWGDGALLKFHEKIKDALDPNGIIAPGKSGVWPQRFRGQNL

SEQ ID NO: 9 - DNA, 1581 bp, Rhodococcus opacus
  source 1..1581: mol_type = genomic DNA
atgacgcgtacccttccccctggcgtttccgacgagcgattcgacgcagcgctccggcgcttccgcgacatcgtgggtgacaagtgggtcctgtcgaccgccgacgaactcgaagcgttccgtgacccgtacccggtcggcgctgccgaggccaatatgccgtcggccgtcgtctcaccggagtcgaccgagcaggtacaggacatcgtccggatcgccaacgagtacggcatccccctctccccggtctcgaccggaaagaacaacggttacggcggcgccgccccgaggctgtcgggttcggtgatcgtcaagaccggtgagcggatgaacagaatcctcgaggtgaacgagaagtacggttacgcactgctcgaacccggggtcacctacttcgacctgtacgagtacctccagagccacgattccggcctgatgctcgactgtcccgatctgggatggggcagcgtcgtcgggaacgccttggaccgcggggtcgggtacaccccgtacggcgatcacttcatgtggcagacgggattggaagtggttctgccgcagggcgatgtgatgcgcaccggcatgggcgcactccccggcagcgacgcgtggcaactgttcccctacgggttcggccccttcccggacggcatgttcacccagtccaatctcgggatcgtcaccaagatggggatcgccctgatgcagcggccgcccgcgtcgcagtcgttcctgatcaccttcgacaaggaggaggatctggagcagatcgtcgacatcatgcttccgctacgcatcaatatggcaccgctgcagaatgttccggtgctgcgcaacatcttcatggacgcggccgcggtgtcgaagcgcaccgagtggttcgacggcgaggggccgatgccggccgaggcggtcgagaggatgaagaaggatctcgatctcggattctggaacttctacggcaccctgtacggacctccgccgctgatcgagatgtactacgggatgatcaaggaggcgttcggcaagattcccggcgcccgtttcttcacacacgaggaacgcgacgatcgcggtgggcatgtgttgcaggacaggcacaagatcaacaacggcatccccagcctggacgaactgcaactgctcgactgggtgcccaacggtgggcacatcgggttttccccggtgtcggcgccggacggtcgtgaggcgatgaaacagttcgagatggtccgcgctcgcgccgacgaatacgccaaggactacgcggcgcaattcatcatcggactccgcgagatgcaccacgtgtgcctgttcatctacgacacggcgatccccgacgctcgtgaggaaatcctgcagatgacgaaggtcctcgtccgggaggccgccgaagccggttacggcgaataccgcacccacaacgccctgatggacgacgtaatggcgacgttcgactggggtgacggtgcgctgctgaagttccacgagaagatcaaggatgccctcgaccccaacggcatcatcgcgcccggcaagtccggtgtgtggccgcagcggttccgtggccagaacctctga

SEQ ID NO: 10 - protein, 526 aa, Rhodococcus opacus
MTRTLPPGVSDERFDAALRRFRDIVGDKWVLSTADELEAFRDPYPVGAAEANMPSAVVSPESTEQVQDIVRIANEYGIPLSPVSTGKNNGYGGAAPRLSGSVIVKTGERMNRILEVNEKYGYALLEPGVTYFDLYEYLQSHDSGLMLDCPDLGWGSVVGNALDRGVGYTPYGDHFMWQTGLEVVLPQGDVMRTGMGALPGSDAWQLFPYGFGPFPDGMFTQSNLGIVTKMGIALMQRPPASQSFLITFDKEEDLEQIVDIMLPLRINMAPLQNVPVLRNIFMDAAAVSKRTEWFDGEGPMPAEAVERMKKDLDLGFWNFYGTLYGPPPLIEMYYGMIKEAFGKIPGARFFTHEERDDRGGHVLQDRHKINNGIPSLDELQLLDWVPNGGHIGFSPVSAPDGREAMKQFEMVRARADEYAKDYAAQFIIGLREMHHVCLFIYDTAIPDAREEILQMTKVLVREAAEAGYGEYRTHNALMDDVMATFDWGDGALLKFHEKIKDALDPNGIIAPGKSGVWPQRFRGQNL

SEQ ID NO: 11 - DNA, 1581 bp, Rhodococcus sp.
  source 1..1581: mol_type = genomic DNA
atgacgcgaacccttccccccggcgtttccgacgagcgattcgacgcggcgctccaacgcttccgcgacatcgtgggcgacaagtgggtcctgtcgaccgccgacgagctcgaagcgttccgcgacccgtacccggtcggcgccgccgaggccaacatgccgtcggccgtcgtctcaccggaatcgaccgagcaggtccaggacatcgtccggatcgccaacgagtacggcatccccctctccccggtctcgaccggaaagaacaacggttacggcggcgccgctccgaggctgtcgggttcggtgatcgtcaagacgggcgagcggatgaaccggatcctcgaggtcaacgagaagtacggttacgcactgctcgaacccggggtcacctacttcgacctctacgactatctgcagagccacgattccggactgatgctcgactgccccgacctggggtggggcagcgtggtcggtaacactctggaccgcggcgtcgggtacaccccgtacggcgaccacttcatgtggcagacgggcctggaagtggtgctgccgcaaggtgatgtgatgcgcaccgggatgggcgcactacccggcagcgacgcgtggcagctgtttccctacgggttcggccccttcccggacggcatgttcacccagtcgaatctcgggatcgtcaccaagatggggatcgccctgatgcagcggccgcccgcgtcgcagtcgttcctgatcaccttcgacaaggaggaggatctggagcagatcgtcgacatcatgctgccgctgcgcatcaacatggctccgctgcagaatgttccggtgctgcgcaacatcttcatggacgcggccgcggtgtcgaagcgcaccgagtggttcgacggtgacgggccgatgccggccgaggcgatcgagaggatgaagaaagacctcgatctcggattctggaacttctacggcaccctgtacggacctccgccgctgatcgagatgtactacgggatgatcaaggaggcgttcggcaagattcccggtgcccgcttcttcacacacgaggaacgcgacgaccgcggtggacatgtgttgcaggacaggcacaagatcaacaacggaatccccagcctggacgaactgcagctcctggactgggtacccaacggcgggcacatcggattctccccggtgtcggcgccggacggtcgcgaggcgatgaaacagttcgagatggtgcgcgctcgcgccgacgaatacgccaaggactacgcggcgcaattcatcatcggactccgcgagatgcaccacgtgtgcctgttcatctacgacacggcgatccccgacgctcgtgaggaaatcctgcagatgacgaaggtcctcgtccgcgaggccgccgacgcgggctacggcgagtaccgcacccacaacgccctgatggacgacgtgatggcgacgttcgactggggcgacggtgcgctgctgaagttccacgagaagatcaaggacgccctcgatcccaacggcatcatcgcgcccggcaagtccggtgtgtggccgcaacggttccgtggccagaacctctga

SEQ ID NO: 12 - protein, 526 aa, Rhodococcus sp.
MTRTLPPGVSDERFDAALQRFRDIVGDKWVLSTADELEAFRDPYPVGAAEANMPSAVVSPESTEQVQDIVRIANEYGIPLSPVSTGKNNGYGGAAPRLSGSVIVKTGERMNRILEVNEKYGYALLEPGVTYFDLYDYLQSHDSGLMLDCPDLGWGSVVGNTLDRGVGYTPYGDHFMWQTGLEVVLPQGDVMRTGMGALPGSDAWQLFPYGFGPFPDGMFTQSNLGIVTKMGIALMQRPPASQSFLITFDKEEDLEQIVDIMLPLRINMAPLQNVPVLRNIFMDAAAVSKRTEWFDGDGPMPAEAIERMKKDLDLGFWNFYGTLYGPPPLIEMYYGMIKEAFGKIPGARFFTHEERDDRGGHVLQDRHKINNGIPSLDELQLLDWVPNGGHIGFSPVSAPDGREAMKQFEMVRARADEYAKDYAAQFIIGLREMHHVCLFIYDTAIPDAREEILQMTKVLVREAADAGYGEYRTHNALMDDVMATFDWGDGALLKFHEKIKDALDPNGIIAPGKSGVWPQRFRGQNL

SEQ ID NO: 13 - DNA, 1575 bp, Rhodococcus koreensis
  source 1..1575: mol_type = genomic DNA
atggtgcgtacccttcccccgggcgtctccgagcaggacttcgatgccgctctggcccgtttccgcgatcttctcggggacaagtgggtgctgtcgaccgcagacgagctcgaagcgttccgcgacccgtacccggtcggcgccgccgaggccaacatgccctcggccgtcgtctcaccggagtcgaccgagcaggttcagggcatcgtccggatcgccaacgagtacggcatccccctctccccggtctcgaccggaaagaacaacggctacggcggcgccgctccgaggctgtcgggttcggtgatcgtcaagaccggcgagcggatgaacaagatcctcgaggtcaacgagaagtacgggtacgcactgctcgaacccggggtcacctacttcgacctgtacgagtacctgcagagccacgactccggcctgatgatcgactgtcccgacctgggatggggcagcgtcgtgggcaacgccctcgaccgcggcgtcgggtacaccccgtacggcgatcacttcatgtggcagacgggactggaagtggttctgccgcagggtgaggtgatgcgcaccgggatgggcgcactacccggcagcgacgcgtggcagctgttcccctacgggttcggccccttcccggacggcttgttcacccagtccaatctcgggatcgtcaccaagatggggatcgccctgatgcagcggccgcccgcatcgcagtcgttcctgatcaccttcgacaaggaggaggatctggagcagatcgtcgacatcatgctgccgctgcgcatcaatatggctccgctgcagaatgttccggtgctgcgcaacatcttcatggatgcggccgcggtgtcgaagcgcaccgagtggttcgacggcgacgggccgatgccggccgaggcgatcgagcggatgaagaaggacctcgacctcggattctggaacttctacggcaccctgtacggcccgccgccgctgatcgagatgtactacgcgatgatcaaggaggcgttcggcaagattcccggtgcccgcttcttcacgcacgaggaacgcgacgaccgcggtggacacgtgctgcaggaccggcacaagatcaacaacggcatccccagcctggacgaactgcaactgctggactgggtgcccaacggcgggcacatcggattctccccggtgtcggcgccggacggccgggaggcgatgaagcagttcgagatggtccgcaaccgcgccaacgaatacaacaaggactacgcggcgcagttcatcatcgggctccgcgagatgcaccacgtgtgcctgttcatctacgacacggcgatccccgaggcccgtgaggaaatcctgcagatgacgaaggtcctcgtccgcgaggccgccgaagcgggctacggcgaataccgcacccacaacgccctgatggacgacgtgatggcgacgttcaactggggcgaaggtgcactgctgaagttccacgagaagatcaaggacgccctcgacccgaacgggatcatggcgcccggcaagtccggtatctggccccagcgattccgtgaccgttaa

SEQ ID NO: 14 - protein, 524 aa, Rhodococcus koreensis
MVRTLPPGVSEQDFDAALARFRDLLGDKWVLSTADELEAFRDPYPVGAAEANMPSAVVSPESTEQVQGIVRIANEYGIPLSPVSTGKNNGYGGAAPRLSGSVIVKTGERMNKILEVNEKYGYALLEPGVTYFDLYEYLQSHDSGLMIDCPDLGWGSVVGNALDRGVGYTPYGDHFMWQTGLEVVLPQGEVMRTGMGALPGSDAWQLFPYGFGPFPDGLFTQSNLGIVTKMGIALMQRPPASQSFLITFDKEEDLEQIVDIMLPLRINMAPLQNVPVLRNIFMDAAAVSKRTEWFDGDGPMPAEAIERMKKDLDLGFWNFYGTLYGPPPLIEMYYAMIKEAFGKIPGARFFTHEERDDRGGHVLQDRHKINNGIPSLDELQLLDWVPNGGHIGFSPVSAPDGREAMKQFEMVRNRANEYNKDYAAQFIIGLREMHHVCLFIYDTAIPEAREEILQMTKVLVREAAEAGYGEYRTHNALMDDVMATFNWGEGALLKFHEKIKDALDPNGIMAPGKSGIWPQRFRDR

SEQ ID NO: 15 - DNA, 1581 bp, Gordonia insulae
  source 1..1581: mol_type = genomic DNA
atgacgcgcacactccctcccggtatcagcgaggaacagttcgacaccgcactgcagcggttccgtgacgtggtcggggacaagtgggtgctgtcggaagcgaccgaactcgagagctttcgcgacccgtatccggttggtgccgaagaagcgttcatcccctcggcggtcatctctcccgagacgaccgaacaggtacaggacatcgtccgtatcgccaacgagtacgggattccgttgtccccggtctccaccggccgcaacaatggctacggcggcgcatccccgcgtctgtcgggctcgatcatcgtcaagaccggcgagcgcatgaacaagatcctcgaggtcaactcgaagtacgcgtacgcacttctcgagccgggcgtgtcgtacttcgacctctacgagcacctgcaggccaacaaccccgacctgatgctggattgtccagaccttggctggggctccgtcgtcggcaacgcgctcgatcgcggggtcggttacacaccctacggagatcacttcatgtggcagaccggcatggaggtcgttctgccgcaaggggatgtcttgcgaacaggcatgggcgccctgccaggcagcgacacgtggcaactgttcccctacggcttcggcccgagccccgacggcatgttcacccagtccaacctcggcatcgtaaccaagatgggtatcggtctgatgcccaagccgcccgcatcgatgtcgttcgtgatcaccttcgacaaggaggaggatctcgagcagatcgttgacatcatgctgccgttgcgtatcaatatggctccgctgcagaatgtcccggttttgcgcaacatcttcatggacgcggctgcggtctcgaagcgcgacgagtggttcgcgggggacggcccgatgccggcggacgcgatcgacaagatgaagaaggaactgaacctcgggtactggaacctgtacgcgacgctgtacgggccgccgccgatgatcgacatgtactactcgatgatcaaggaggccttcggccaggtccctggcgcccagttctttacgcacgaagaacgtcacgatcgcggtggccacgttctcgccgaccgtcacaagatcaacaacggcatcccgagtctggacgaactgcagttgctggactgggtccccaatggcggtcacatctgcttctcgccagtgtcggcgcccgatggcaaagaggcgatgaagcagttcgagatggtgcgggcacgtgccaacgagtacaacaaggactacgccgcgcagttcatcgtcggcctccgcgagatgcatcacgtgtgtctgttcgtctacgacaccagcgatccggacattcgtcagcaggccctcgacatgactcgcgtactcgtgcgggaagcggccgaatccggatatggcgagtatcggacccacaacgccttgatggacgacgtgatggcgacgttcaactggggtgacggtgcacttctgaagttccacgagaagatcaaggacgccctcgaccccaacggcatcttggcgccgggtaagtcgggcgtgtggcccgaacgcttccgcggcggcaaccactag

SEQ ID NO: 16 - protein, 526 aa, Gordonia insulae
MTRTLPPGISEEQFDTALQRFRDVVGDKWVLSEATELESFRDPYPVGAEEAFIPSAVISPETTEQVQDIVRIANEYGIPLSPVSTGRNNGYGGASPRLSGSIIVKTGERMNKILEVNSKYAYALLEPGVSYFDLYEHLQANNPDLMLDCPDLGWGSVVGNALDRGVGYTPYGDHFMWQTGMEVVLPQGDVLRTGMGALPGSDTWQLFPYGFGPSPDGMFTQSNLGIVTKMGIGLMPKPPASMSFVITFDKEEDLEQIVDIMLPLRINMAPLQNVPVLRNIFMDAAAVSKRDEWFAGDGPMPADAIDKMKKELNLGYWNLYATLYGPPPMIDMYYSMIKEAFGQVPGAQFFTHEERHDRGGHVLADRHKINNGIPSLDELQLLDWVPNGGHICFSPVSAPDGKEAMKQFEMVRARANEYNKDYAAQFIVGLREMHHVCLFVYDTSDPDIRQQALDMTRVLVREAAESGYGEYRTHNALMDDVMATFNWGDGALLKFHEKIKDALDPNGILAPGKSGVWPERFRGGNH

SEQ ID NO: 17 - DNA, 1581 bp, Rhodococcus sp.
  source 1..1581: mol_type = genomic DNA
atgacgcgaacccttcccccaggcgtttccgacgagcgattcgacgcggcgctccagcgcttccgcgaggtcgtcggcgacaaatgggtcctgtcgaccgcagacgaactcgaagcgttccgcgacccgtacccggtcggcgccgccgaggccaacatgccgtcggccgtcgtctcaccggagtcgaccgagcaggtccaggacatcgtccggatcgccaacgagtacggcatccccctctccccggtctcgaccggaaagaacaacggttacggcggcgccgccccgaggctgtcgggttcggtgatcgtcaagaccggcgagcggatgaaccgaatcctcgaggtcaacgagaagtacggctacgcactcctcgaacccggggtcacctacttcgacctgtacgagtacctccagagtcacgattccggcctgatgctcgactgtcccgacctgggatggggcagcgtggtgggaaacaccctggaccgcggcgtcgggtacaccccgtacggcgatcacttcatgtggcagacgggactggaagtggtcctgccgcagggcgaagtgatgcgcaccgggatgggcgcactgcccggcagcgacgcgtggcaattgttcccctacgggttcggccccttcccggacggcatgttcacccagtcgaatctcgggatcgtcaccaagatggggatcggcctgatgcagcggccgcccgcgtcgcagtcgttcctgatcaccttcgacaaggaggaggatctcgagcaaatcgtcgacatcatgctgccgctgcgcatcaatatggctccgctgcagaatgttccggtgctgcgcaacatcttcatggacgcggccgcggtgtcgaagcgcaccgagtggttcgacggcgacgggccgatgccggccgaggcgatcgagcggatgaagaaggatctcgatctcgggttctggaacttctacggcaccctgtacggacctccgccgctgatcgagatgtactacggaatgatcaaggaggcgttcggcaagattcccggtgcccgcttcttcacacacgaggaacgtgacgaccgcggtggacatgtgttgcaggacaggcacaagatcaacaacggcatccccagcctggacgaactgcaactcctggactgggtgcccaacggcgggcacatcggattctccccggtgtcggcgccggacggtcgcgaggcgatgaagcagttcgagatggtccgcgctcgcgccgacgaatacgccaaggactacgccgcgcaattcatcatcgggctccgcgagatgcaccacgtgtgcctgttcatctacgacacggcgatccccgatgctcgtgaggaaatcctgcagatgacgaaggtcctcgtccgcgaggccgccgaagccggctacggcgaataccgcacccacaacgccctgatggacgacgtgatggcgacgttcgactggggcgacggtgcgctgctgaagttccacgagaagatcaaggacgccctcgaccccaacggcatcatcgcacccggtaagtccggtgtgtggccgcagcggttccgtggccagaacctctga

SEQ ID NO: 18 - protein, 526 aa, Rhodococcus sp.
MTRTLPPGVSDERFDAALQRFREVVGDKWVLSTADELEAFRDPYPVGAAEANMPSAVVSPESTEQVQDIVRIANEYGIPLSPVSTGKNNGYGGAAPRLSGSVIVKTGERMNRILEVNEKYGYALLEPGVTYFDLYEYLQSHDSGLMLDCPDLGWGSVVGNTLDRGVGYTPYGDHFMWQTGLEVVLPQGEVMRTGMGALPGSDAWQLFPYGFGPFPDGMFTQSNLGIVTKMGIGLMQRPPASQSFLITFDKEEDLEQIVDIMLPLRINMAPLQNVPVLRNIFMDAAAVSKRTEWFDGDGPMPAEAIERMKKDLDLGFWNFYGTLYGPPPLIEMYYGMIKEAFGKIPGARFFTHEERDDRGGHVLQDRHKINNGIPSLDELQLLDWVPNGGHIGFSPVSAPDGREAMKQFEMVRARADEYAKDYAAQFIIGLREMHHVCLFIYDTAIPDAREEILQMTKVLVREAAEAGYGEYRTHNALMDDVMATFDWGDGALLKFHEKIKDALDPNGIIAPGKSGVWPQRFRGQNL

SEQ ID NO: 19 - DNA, 1581 bp, Rhodococcus erythropolis
  source 1..1581: mol_type = genomic DNA
atgacccgaaccctcccccccggtctgtccgaggaacaattcgacgcggcgctcgcgcgtttccgtgacgtggtcggcgacaagtgggtgctgtcctctgccgatgagctcgcgggctttcgggacccgtatccggtgggcgcagccgaagcgtacgtgccttcggcggtggtatcgccggaatcgaccgagcaggtacaggcgatcgtccggattgccaacgagttcggaattccgttgtcgccggtgtcaaccgggaagaacaacggctacggtggcgcttcgccgcgcttgtcgggttcggtgatcgtcaagaccggtgagcggatgaacaagatcctcgaggtcaacgagaagtacgcctacgcgctgctcgagcccggggtcacctacttcgatctgtacgagtacctcgagagcaacaacctgaacctgatgctcgactgtccggacctcggctggggcagcgtcgtcggcaacgccctggaccgcggtgtcggctacaccccgtacggcgaccacttcatgtggcagaccggactggaagtggttctgccgcagggtgatgtgatgcgcaccgggatgggcgccctgcccggcagtgatacctggcagctgttcccgtacggcttcgggccgagcccggacggcatgttcacccagtccaacctgggcatcgtcaccaagatgggtatcgccctgatgcccaaaccgccggcgtcgcagtcgttcctgatcaccttcgacaaggaggaagatctcgagcagatcgttgacatcatgttgccgctgcgcatcaacatggctccgctgcagaacgtgccggtactacgcaacatcttcatggacgccgccgcggtgtccaagcgttcggagtggttcgacggtgacgggccgatgcctgccgaggcgatcgagaagatgaagcaggacctgaacctcgggtactggaacttctacggcaccctctacggtccaccgccgatgatcgagatgtactactcgatgatcaaggaagctttcgggcagatccccggagcccggttctacacccacaccgagcgtgacgaccgcggcggccacgtgttgcaggaccggcacaagatcaacaacggcatccccagcctggacgagctgcagctgctcgactgggttcccaacggcggtcacatctgcttctccccggtctcggccccggacggcagggaggcgatgaagcagttcgagatggtccgcgaccgcgcgaacgagtacagcaaggactacgccgcccagttcatcgtcggcctacgcgagatgcaccacgtgtgcttgttcgtttacgacacctccatcccggacgtgcgtgaggaagccctgcagatgacgaaggtcctcgtccgtgaggccgccgaggagggatacggcgaataccgcacgcacaacgctctgatggacgacgtcatggccacgttcaactggggtgacggcgccctgctcaagttccacgagaaggtcaaggacgccctcgaccccaacggcatcatcgcccccggtaagtcgggtgtgtggccgcaacggttccgtggtcagaacctctga

SEQ ID NO: 20 - protein, 526 aa, Rhodococcus erythropolis
MTRTLPPGLSEEQFDAALARFRDVVGDKWVLSSADELAGFRDPYPVGAAEAYVPSAVVSPESTEQVQAIVRIANEFGIPLSPVSTGKNNGYGGASPRLSGSVIVKTGERMNKILEVNEKYAYALLEPGVTYFDLYEYLESNNLNLMLDCPDLGWGSVVGNALDRGVGYTPYGDHFMWQTGLEVVLPQGDVMRTGMGALPGSDTWQLFPYGFGPSPDGMFTQSNLGIVTKMGIALMPKPPASQSFLITFDKEEDLEQIVDIMLPLRINMAPLQNVPVLRNIFMDAAAVSKRSEWFDGDGPMPAEAIEKMKQDLNLGYWNFYGTLYGPPPMIEMYYSMIKEAFGQIPGARFYTHTERDDRGGHVLQDRHKINNGIPSLDELQLLDWVPNGGHICFSPVSAPDGREAMKQFEMVRDRANEYSKDYAAQFIVGLREMHHVCLFVYDTSIPDVREEALQMTKVLVREAAEEGYGEYRTHNALMDDVMATFNWGDGALLKFHEKVKDALDPNGIIAPGKSGVWPQRFRGQNL

SEQ ID NO: 21 - skipped

SEQ ID NO: 22 - protein, 535 aa, unidentified
MTSSSTRVLPPGLRPEDFDAAIARFRAVVGDKYVLTDDVDLARFRDPYPVGSEVAAGASAVVAPESAEQVQEIVRIANELGVPLSPVSTGKNNGYGGAQPRLSGAVVVNTGERMNRILEVNERFGYALLEPGVSYFDLYEYLEANAPGLMLDCPDLGWGSVVGNCLDRGVGYTPYGDHFLWQTGMEVVLPDATIMRTGMGAVPGSNSWQLFPYGFGPFPDGMFTQSNLGIVTKMGIALMPKPPGATSFLVAFENEDDLAQVVDIMLPLRINMAPLQNVPVLRNIILDAGVVSRRTEWYDGDGPLPDEAIEKMKKTLDLGYWNLYGTVYGPPPIMEMYLGMIKEAFLQVPGARFWTHHDRPDDMALDRGAHTLHDRHKINNGIPSLDEMKLMEYLPNGGHICFSPVSAPDGKDALRQAKMVKARSDEYAKDYAAQFIVGLREMHHIGLFLYDTSKPEERQGALDMARVLIDEAAAEGYGEYRTHNALMDQVMGTYNWGDGALLKFHEAVKDALDPNGIIAPGKSGIWPRKYRGQGL

SEQ ID NO: 23 - DNA, 1440 bp, synthetic construct
  source 1..1440: mol_type = other DNA
atggcacgcctgaatcgcaatgatccgcagctggttggtaccctgctgccgacccgtattgaagcagatctgtttgatctggaagtggatggtgaaattccgaaaagcattaatggtaccttttatcgtaacaccccggaaccgcaggtgaccccgcagaaatttcatacctttattgatggtgacggtatggccagtgcatttcattttgaagatggccatgttgattttattagtcgttgggttaaaaccgcccgctttaccgccgaacgcctggcccgtaaaagtctgtttggcatgtatcgtaatccgtataccgatgataccagcgttaaaggtctggatcgtaccgtggcaaataccagcattattagccatcatggcaaagtgctggcagtgaaagaagatggcctgccgtatgaactggaccctcgtaccctggaaacccgcggtcgctttgattatgatggtcaggtgaccagtcagacccataccgcccatccgaaatatgatccggaaaccggcgatctgctgtttttcggcagcgccgccaaaggtgaagcaaccccggatatggcctattatattgtggataaacatggtaaagtgacccatgaaacctggtttgaacagccgtatggtgcattcatgcatgattttgcaattacccgcaattggagtatttttccgattatgccggcaaccaatagcctgagtcgtctgaaagccaaacagccgatctatatgtgggaaccggaactgggtagctatattggcgttctgccgcgtcgcggccagggcagccaaattcgctggctgaaagcaccggcactgtgggtgtttcatgtggtgaatgcatgggaagttggcaccaaaatctatattgatctgatggaaagcgaaattctgccgtttccgtttccgaatagtcagaatcagccgtttgcaccggaaaaagccgttccgcgcctgacccgttgggaaattgatctggatagcagcagcgatgaaattaagcgcacccgcctgcatgatttctttgcagaaatgccgattatggattttcgttttgccctgcagtgcaatcgctatggctttatgggtgttgatgatccgcgtaaaccgctggcccatcagcaggccgaaaaaatttttgcatataatagtctgggcatctgggataatcatcgtggcgattatgatctgtggtatagcggcgaagccagtgcagcccaggaaccggcctttgtgccgcgtagcccgaccgctgcagaaggcgatggttatctgctgaccgttgtgggtcgcctggatgaaaatcgcagcgatctggttattctggatacccaggatattcagagcggcccggtggcaaccattaagctgccgtttcgcctgcgcgccgcactgcatggttgttgggtgccgcgtccgtaataa

SEQ ID NO: 24 - protein, 478 aa, Pseudomonas nitroreducens
MARLNRNDPQLVGTLLPTRIEADLFDLEVDGEIPKSINGTFYRNTPEPQVTPQKFHTFIDGDGMASAFHFEDGHVDFISRWVKTARFTAERLARKSLFGMYRNPYTDDTSVKGLDRTVANTSIISHHGKVLAVKEDGLPYELDPRTLETRGRFDYDGQVTSQTHTAHPKYDPETGDLLFFGSAAKGEATPDMAYYIVDKHGKVTHETWFEQPYGAFMHDFAITRNWSIFPIMPATNSLSRLKAKQPIYMWEPELGSYIGVLPRRGQGSQIRWLKAPALWVFHVVNAWEVGTKIYIDLMESEILPFPFPNSQNQPFAPEKAVPRLTRWEIDLDSSSDEIKRTRLHDFFAEMPIMDFRFALQCNRYGFMGVDDPRKPLAHQQAEKIFAYNSLGIWDNHRGDYDLWYSGEASAAQEPAFVPRSPTAAEGDGYLLTVVGRLDENRSDLVILDTQDIQSGPVATIKLPFRLRAALHGCWVPRP

SEQ ID NO: 25 - DNA, 1815 bp, synthetic construct
  source 1..1815: mol_type = other DNA
atggcacatattcatgatctggcaccggaagtgagcaattatagcagtggtcgtctgaccccgccgaccccggttcgcttccctcgtaccccggtgttcgcaagcatgaataaaccgtgccgcttcgaaggcgatgtgttcgatctggaagttagcggtgccattccgccggatattgatggcaccttcttccgcgtgcagccggatcatcgcttcccgccgctgttcgaagatgatattcacttcaatggtgatggtagtgttaccgcaattcgtattagcggcggtcatgcagatctgcgccagcgctatgtgcgcaccgaacgctatctgctggaaaccagagcccgtcgcagtctgttcggccgctatcgtaatccgtggaccgataatgaaagcgttcgcggtgtgattcgtaccgcaagtaataccaatgttgtgttctggcgcggtgcactgctggccatgaaagaagatggcccgccgttcgccatggaccctgtgaccctggaaaccttaggtcgttatgacttcgaaggtcagattctgagtccgaccttcaccgcccatccgaaaattgatccggataccggcgaaatggtgtgcttcgcatacgaagccggtggtgatggcagtgattgtagcgttgatgtggcagtgtggaccgttgatgcagatggtaaaaaagtggaagaatgctggtataaagccccgttcgcaggcatgattcatgattgtggcattaccaaaaattgggtggttctgccgctgaccccgattaaaatggatctggaacgtatgaaacgtggtggcaataaattcgcatgggaccctagtgaagatcagtggtatggtgtggttccgcgtcgcggtgccaaaagtgatgatattatctggttccgcgccgataatggcttccatggccatgttgcaggctgctatgaactgccgagtggcgaaattgtgttcgatcttaccgtggccgatggtaatgtgttcttcttcttcccgccggatgataatattaccccgccggccgatggtgtggcaaaacgtaatcgcctgagtagcccgaccgttcgttggatcttcgatccgaaagcaaaaaaaagcgcaattcgcaccgaagcagccggcgatgccgatatctgggttgccgatgaacgcgtgaaaccggcactgacctggccgaccaatggcgaattcagccgtattgatgatcgctatgtgaccaaaccgtatcgccacttctggcaggcagtggtggaccctacccgcccgtatgacttcgagaaatgtggtccgccggcaggcggtctgttcaattgcctgggccattatacctggagtgatcagaattatcatcatggtcataataccggtgatccgagcggtgatggtcgcagcaatggcagtgcagaagaagcaaccgcaggtaaattcggtctgcaggatgtgtacttcgcaggtccgaccatgaccttccaggaaccgaccttcattccgcgccagggtgcagccgaaggtgaaggttatctgattgcactgctgaatcatctggatgaactgcgtaatgatgtggtgatcttcgaagcacgtaatctgggtaaaggtccgctggcagttattcatctgccgctgaaactgaaactgggcctgcatggcaattgggtggatagtcgtgaaattgaagcatggcgtcgtcgccgtgccgaaaatggcgatgttggtccgctgcgtgtggccaaagaaccgctgccgtggcagaaaaaattcgcagcagcagcccagaatggtagtaatggtgtttaataa

SEQ ID NO: 26 - protein, 603 aa, Thermothelomyces thermophilus
MAHIHDLAPEVSNYSSGRLTPPTPVRFPRTPVFASMNKPCRFEGDVFDLEVSGAIPPDIDGTFFRVQPDHRFPPLFEDDIHFNGDGSVTAIRISGGHADLRQRYVRTERYLLETRARRSLFGRYRNPWTDNESVRGVIRTASNTNVVFWRGALLAMKEDGPPFAMDPVTLETLGRYDFEGQILSPTFTAHPKIDPDTGEMVCFAYEAGGDGSDCSVDVAVWTVDADGKKVEECWYKAPFAGMIHDCGITKNWVVLPLTPIKMDLERMKRGGNKFAWDPSEDQWYGVVPRRGAKSDDIIWFRADNGFHGHVAGCYELPSGEIVFDLTVADGNVFFFFPPDDNITPPADGVAKRNRLSSPTVRWIFDPKAKKSAIRTEAAGDADIWVADERVKPALTWPTNGEFSRIDDRYVTKPYRHFWQAVVDPTRPYDFEKCGPPAGGLFNCLGHYTWSDQNYHHGHNTGDPSGDGRSNGSAEEATAGKFGLQDVYFAGPTMTFQEPTFIPRQGAAEGEGYLIALLNHLDELRNDVVIFEARNLGKGPLAVIHLPLKLKLGLHGNWVDSREIEAWRRRRAENGDVGPLRVAKEPLPWQKKFAAAAQNGSNGV

SEQ ID NO: 27 - DNA, 1455 bp, Pseudonocardia hierapolitana
  source 1..1455: mol_type = genomic DNA
atgaccaggttcccgcagcgggagggcgcgagcgcgttcgaggcgccgatgcgcgtcgaggccgacatcgccggcctcgaggtcgtgcagggcgaggtgcccgaacacctgtccggcacctactaccgcgtggtcaccgaccggcagtggccgcctgccgtgctcccggacgtgccgacgttcaacgacgacggcatggccatgtacttccgcttccacggcggccgggtcgacttccgcagccgctacgtgcgcacgccgcgctacgaggccgaggcgaaggccgggcgggcgctcttcggggcctaccgcaacccgctcaccgacgacccgtccgtcgccgggatctcccgcgggctgggtaacaccaacgtcttctaccatggcgggaagctgtacgcgtccaaggaggacagcgcgccgatcctgctcgacccgatgagcctggagacgatcggcgagtacacgttcgagggcacgctcacgtcccggacgtgcaccgcgcacccgaaggtggatccgcgcaccggcgagatggtgttcttcggctacgccgccaagggcgagaccaccccggacatcgcctactacgaggccgaccaggacggccggatcatccacgagacctggatccgcgcgccgtactcgagcatggtgcacgactgggccgtcacccagaacttcgtggtcttcccgatcatcccgatgcgcagcgagtacgcgtggctggaagcgcggcagccgcacttccagtgggacccggaccgagacgtctacctcggcgtcctgccccgcaagggcagcgccgaccaggtgcgctggctgcgcgggtccaaccggttcgcgagccacatcctcggcgcgtgggacgacgggcgccacatccacatcgagacgccggtcggccggagcaacttcttcccgttcttccccgacgtgaccggcgcgccgtacgacccggtccgcgcccgcgggtacctgtcccgctggacgatcgacatgggttccgacggcgggacgttcaccgagcagcggctgaccgacgtcgcgggcgagttcccgcggatggacgaccgcttcgagacgctgccctaccggcgcggcttcctcgcgctcaaccacgtgcccggcgagcagcggccgggcgcgggcttccgctggatcggcgcgttcgacaccaccggcgcggcgccgccgcagatctactaccccggcgacgactgctcggtggccgagccgctcttcgtccccggcggcgacgacgcgcccgagggccacggctacgtgttcgtcgtcgtagggcgccaccaggagatgcgcagcgacctggtgatcctcgacgcccagcggctcgacgccccgcccgtcgccacggtggcgctgcccatccgcatccgccgcggcctgcacggcaactgggtgagcagcacggagctcgccgcccgtaccgcctga

SEQ ID NO: 28 - protein, 484 aa, Pseudonocardia hierapolitana
MTRFPQREGASAFEAPMRVEADIAGLEVVQGEVPEHLSGTYYRVVTDRQWPPAVLPDVPTFNDDGMAMYFRFHGGRVDFRSRYVRTPRYEAEAKAGRALFGAYRNPLTDDPSVAGISRGLGNTNVFYHGGKLYASKEDSAPILLDPMSLETIGEYTFEGTLTSRTCTAHPKVDPRTGEMVFFGYAAKGETTPDIAYYEADQDGRIIHETWIRAPYSSMVHDWAVTQNFVVFPIIPMRSEYAWLEARQPHFQWDPDRDVYLGVLPRKGSADQVRWLRGSNRFASHILGAWDDGRHIHIETPVGRSNFFPFFPDVTGAPYDPVRARGYLSRWTIDMGSDGGTFTEQRLTDVAGEFPRMDDRFETLPYRRGFLALNHVPGEQRPGAGFRWIGAFDTTGAAPPQIYYPGDDCSVAEPLFVPGGDDAPEGHGYVFVVVGRHQEMRSDLVILDAQRLDAPPVATVALPIRIRRGLHGNWVSSTELAARTA

SEQ ID NO: 29 - DNA, 1485 bp, synthetic construct
  source 1..1485: mol_type = other DNA
atgagcctgttcaatcgcaatcatccggtgttcagcggtggcaatgcaccggttcgcattgaaaccgatgcaatctgtctggaaaccgatggcgcactgccggatgaactgaaaggcagctacttccgtctgaccgcagatccgcagttcccgccgcgcctgcgtcctgttctggtggaagcagatggtcatgttggtgcattccacttccgtgataatggtcaggtggattatgttggcaaatgggtgcgtaccgaacgcttcctgcgcgaacgtgccgcccgtcgtgccttattcggtacctatcgtaatcgcttcaccgatgaagaaagcgtgctggatagcgatcgtaccaccgcaaataccgccttcatgttccatcatcataaattcttcgccctgaaagaagatggtctgccgtatcagctggaccctgaaaccttagataccattggccgctatgacttcggtggcaccgttagtgcaaaaagcctgagtgcacatccgaaagtggatagccgtaccggtgaactgattacccatagtggtcaggccaaaggcgaaggtaccccggatattgcctattatgtgttcgataaaaacggcaccaaaaccgttgaacgttggttcgaagccccgtatagcagtattgtgcatgatattgccattacccaggattgggtggtgctgccgattatgccggccgttgtggaagaagaacgcctgcgcgcaggtggcgcaacctattggtggaaaccggaactgggcaatcatattgcagtgttccgccgtgatggtaccggtgatgttcgctggttccgtaccaaagccacctatgccttccatgttgttaatagctatcaggatggcgatcgtctgattattgatgttatggatgcagaagaattcccgatgtggtggccgcgcccggaacaggttgccgcactgcgtagtggcgaaattaaacgtgataaattcgtggcacagctgacccgctggaccattgatctggccggtgatagtgatgatattgaacgcgaactgctgcatccgtgggaagcagaaatgccgcgcattgatgatcgcttcgcaggtcagccgtatcgttatgccgtgtatggcgttgatgatccgagcttcccgctggcccatggcctggctgaactgggcgtgaatcataatagtgttggctggtgggatcatcagacccgtaccctgaccagctggtataccggcccgaatagtagtgtgggtgaaccggtgttcaccccgcgtagtccggatgcaccggaaggcgatggcttcattctggccgtggtgcagcgcctggccgaacagcgtagcgaactggttgttattgatacccgtgatgttgccgccggcccgattgccagcgttcatgccccgcatcgtctgaaaaatgccattcataatctgtggattgatcaggaacagctgaatgaaggcggtgaaaaagcataataa

SEQ ID NO: 30 - protein, 493 aa, Sphingomonadales bacterium
MSLFNRNHPVFSGGNAPVRIETDAICLETDGALPDELKGSYFRLTADPQFPPRLRPVLVEADGHVGAFHFRDNGQVDYVGKWVRTERFLRERAARRALFGTYRNRFTDEESVLDSDRTTANTAFMFHHHKFFALKEDGLPYQLDPETLDTIGRYDFGGTVSAKSLSAHPKVDSRTGELITHSGQAKGEGTPDIAYYVFDKNGTKTVERWFEAPYSSIVHDIAITQDWVVLPIMPAVVEEERLRAGGATYWWKPELGNHIAVFRRDGTGDVRWFRTKATYAFHVVNSYQDGDRLIIDVMDAEEFPMWWPRPEQVAALRSGEIKRDKFVAQLTRWTIDLAGDSDDIERELLHPWEAEMPRIDDRFAGQPYRYAVYGVDDPSFPLAHGLAELGVNHNSVGWWDHQTRTLTSWYTGPNSSVGEPVFTPRSPDAPEGDGFILAVVQRLAEQRSELVVIDTRDVAAGPIASVHAPHRLKNAIHNLWIDQEQLNEGGEKA

SEQ ID NO: 31 - DNA, 1506 bp, Rhodococcus sp.
  source 1..1506: mol_type = genomic DNA
atgattcacgtcgcgtcaccggacgaccgtgggtcaacggttcatccgaaggagacgcacgtgattcgctttcccgtcaccgacgatccgttcgacgccccgatgcgtgtcgaggccgatatcaccgatctcgaatgcacccagggtgccgtgcccgaacacctcgacggcacttactaccgcgttgtagtcgacagacactacccctccatggtcgacaacgatctcgcccttttcaactccgacggtatggcaatgtctttcaccttcagcaagggacgtgtggacttcaaaactcggtatgtgcgcacgccacgattcgtagccgaggaaaaagctggcaaatcactgttcggcgcctaccgtaatgcctcgaccgacgacccatcggtcgcagggatccgtcgtgggctggcgaacaccaatgtcttctttcacggtgggcgcctgtatgcctccaaggaggacagtccgcccatacttctggaccccttcagcctcgagacactcggagagtacgacttccaaggtgccctgacctccgaaacatctacagcccacccgaaagtcgatccacgcaccggagaaatggtgttcttcgggtacggagccaagggtgaagccacacctgatatcgcgtactacgaagccgacgcatccgggcagatcatccatgaggcctggatcactgctccctactcatgcatgatccatgactttgcagttactcagaactacgtcatcttcccaatcgtgccgctacgcagcagcactgagtggatcgcacaaggaaacaaccagtatttctgggatcccgatgaggacgtatatctgggagtactcccacgcaaaggaagcggaaaggaccttcgctggttccgtggcagcaaccgattcgccagtcacatcatgggtgccttcgacgacgggcgacacatctacatcgacacaccggtcgccgagacgagctactttccctggttccccgacatcagtggtgcctcttacgatcctgaccgcagcaaaacacatctgtcacgctggacgatcgacctcgccgattccaccggcggtttcaccgagcggcgactgatgaccgccgccggcgaattcccccgaatggacgaccgtcaggaaacgctcgcttacgactggggggtactggcgatcaatgatctacccggcagccccaagcccggcccgggatttcgctggatcggtgccgtcgacctaccctccggctcgaccaagttctacgacccgggtccgaacgctaccgttggagaaccgatcttcgtcccgggccaggcggatgcaaaaccgggccacgggtacgtcttcgtcgtcgttgctcgccgcgacctcatgcgcagcgacctggtcatactcgacgcgcagaacctcgacaaaccgccggttgccaccctgtcgctaccaatacggatcaagtcgggtctccacggtaactgggtcggggcagacgaactggctggcaggaccgcgtga

SEQ ID NO: 32 - protein, 501 aa, Rhodococcus sp.
MIHVASPDDRGSTVHPKETHVIRFPVTDDPFDAPMRVEADITDLECTQGAVPEHLDGTYYRVVVDRHYPSMVDNDLALFNSDGMAMSFTFSKGRVDFKTRYVRTPRFVAEEKAGKSLFGAYRNASTDDPSVAGIRRGLANTNVFFHGGRLYASKEDSPPILLDPFSLETLGEYDFQGALTSETSTAHPKVDPRTGEMVFFGYGAKGEATPDIAYYEADASGQIIHEAWITAPYSCMIHDFAVTQNYVIFPIVPLRSSTEWIAQGNNQYFWDPDEDVYLGVLPRKGSGKDLRWFRGSNRFASHIMGAFDDGRHIYIDTPVAETSYFPWFPDISGASYDPDRSKTHLSRWTIDLADSTGGFTERRLMTAAGEFPRMDDRQETLAYDWGVLAINDLPGSPKPGPGFRWIGAVDLPSGSTKFYDPGPNATVGEPIFVPGQADAKPGHGYVFVVVARRDLMRSDLVILDAQNLDKPPVATLSLPIRIKSGLHGNWVGADELAGRTA

SEQ ID NO: 33 - DNA, 1452 bp, Amycolatopsis alkalitolerans
  source 1..1452: mol_type = genomic DNA
atgaccccgcccacccgtttccccgacgacccgctgttcgccgggttcaacgcgccggtccgcgtcgaggccgacatctacgatctcgaggtctcgcaaggacaggtgcccgaacagctcgacggcacctactaccgggtggtcgcggaccggcagtggccgccgaaggtggaccacgacatcgggttcaacgccgacggcatggtgatgtcgttccggttcacccgcggccacgtggacttcaagagccggtacgtgcgcacgccgcggttcgaagccgagcgcacggcgcggcgggcgctgttcggcgcgtaccgcaacgcgttcaccgacgatccctcggtgctcggggtgaaccggaccctggccaacaccaacgtgttctggcacggcgggaagctattggcgtccaaagaggacggaccgccgatccggatcgacccggacacgctggaaaccgccggggtgcacaccttcgacggggcgctgagctcgccgacctcgaccgcgcacccgaagttcgacccccgcaccggcgaaatggtcttcttcggctacgccgcgaaaggcgaaggcacgcccgacatcgtctactacgaggccgaccgcgccgggaacctgattcatgaaacctggttccaggccccgtattcgagtatggtgcacgatttcgcggtgacccagaacttcgtggtgttcccgatcatcccgctcaccggcgacctcgaccgcatccgcagggggcagccgctgttcgcgtgggatccgggcaaggacgtctatctcggcgtgctgccgcgcaagggccgggtcgaggatctgcgctggtaccgcgggggtacgcggttcgccagccacatcctcggcgcgttcgacgacggcaggcacatccacctcgacaccccggtgtcggagacgaactacttcccgttcttcccggacctctccggcgcgcctgtcgatccggagaagaccaagggttacctgtcccggtggacgatcgacacccagggtgagtcgaccatgttcaccgaagagcggctcaccgggcacgcgggcgagttcccgaggatggacgaccggttcgagacactgccgtactcgtggggcgtgctgggcatgtacgacgtgccgggggagcgcaggcccggtcgcgggttccgctgggtgggcaccgtcgacctcgcgcgcaagcgcacggccacgcattacgtcggcgacgcctcgtcggccggggagcccctgttcgttccggcgcacgacgaggccgggcacggcgagggctatgtgatggtggtggtcgggcggcacgacgagatgcgcagcgacctgctcatcctcgacgccgcccgcatcggcgcgccaccggtcgcgacggtgaagctgccgatccggatcgcctacggcctgcacggcaactgggtgccacgcaccgagctcgagcgtgcctga

SEQ ID NO: 34 - protein, 483 aa, Amycolatopsis alkalitolerans
MTPPTRFPDDPLFAGFNAPVRVEADIYDLEVSQGQVPEQLDGTYYRVVADRQWPPKVDHDIGFNADGMVMSFRFTRGHVDFKSRYVRTPRFEAERTARRALFGAYRNAFTDDPSVLGVNRTLANTNVFWHGGKLLASKEDGPPIRIDPDTLETAGVHTFDGALSSPTSTAHPKFDPRTGEMVFFGYAAKGEGTPDIVYYEADRAGNLIHETWFQAPYSSMVHDFAVTQNFVVFPIIPLTGDLDRIRRGQPLFAWDPGKDVYLGVLPRKGRVEDLRWYRGGTRFASHILGAFDDGRHIHLDTPVSETNYFPFFPDLSGAPVDPEKTKGYLSRWTIDTQGESTMFTEERLTGHAGEFPRMDDRFETLPYSWGVLGMYDVPGERRPGRGFRWVGTVDLARKRTATHYVGDASSAGEPLFVPAHDEAGHGEGYVMVVVGRHDEMRSDLLILDAARIGAPPVATVKLPIRIAYGLHGNWVPRTELERA

SEQ ID NO: 35 - DNA, 1455 bp, Pseudonocardiaceae bacterium
  source 1..1455: mol_type = genomic DNA
atggttactcgtttccccgatcagccagggttcaccggtttcgacgcccccatgcgcgtcgaagcggatctgatggatgtcgaggtatcgcagggggcgatacccgaaaacctcgaggggacctactaccgcgtcgtcgccgatttccagtggccgccgaaggtggacggcgacttcttcttcaacggcgacgggatggtgatgtccttccgcttctccggcgggcacgccgattttaagagccggtacgtgcgcaccccccggttcgtcgcgcaagccaaggaacgcagggcgttgttcggcgcctaccgcaacccgtacaccgacgatccctcggtagccggcatgagccgcgggctcgccaacacgaatgtgttctggcacgggggaaagctcctgtcgtccaaagaggacagtccgccgatgatgatcgacccggataccctcgagacgatcggcgagcacaccttcgacggagcgctcacctcgcaaacctcgaccgcacacccgaaaatcgacccgcgtaccggggaaatggtcttcttcgggttcgcggcgaagggcgagaccacccctgatatcgcctactacgaggcggacagccgcggcgtggtgatccacgagacctggttgcgcgcaccgtattcaagcatggtgcacgacttcgcggtcacccagaacttcgtcgtgttcccgatcatcccgttgacaagtgatctcgaacggttgaaggcgggcggcccgcattacgcctgggatccgcacaaggacgtctatctcggtgtgctgccacgcaagggcaacggcgcggacatccgctggtaccgcggctcgaaccggttcgccagccacatcatgggcgcacacgacgacggccggcacatcttcatcgacaccccggtcagcgagtcgaacttcttcccgttcttcccggacctttccggtgcgggttacgaagcggagaaggccaaggggtatctgtcccgctggacgatcgacacgatgggcgagtcgaacggcttcaccgagacgaaaatcgcgcgctatccgggggaattcccgcggatggacgaccgcagggaaacgctgccgtacaactggggtgtgctggcactctccgacgtacccgagcaggaatcgccggggcgcggattccggtggatatccagcatcgacctgaacaccgggcacagccagatctactacccgggccacgactgctcggtggctgaaccgctgttcgtgcccgcgcacgacaacgccgaagagcgcgagggatacgttttcgtggtcattggccggcacgaggagatgcgcagcgatctggtgatcctggacgccgcgcatctcgacgcaccgcccgtcgcgaccctgaagatgccccttcgaattcgatccgggttgcacggtaactgggtgtcggcgaccgaactggcgagagagcgcacggcctga

SEQ ID NO: 36 - protein, 484 aa, Pseudonocardiaceae bacterium
MVTRFPDQPGFTGFDAPMRVEADLMDVEVSQGAIPENLEGTYYRVVADFQWPPKVDGDFFFNGDGMVMSFRFSGGHADFKSRYVRTPRFVAQAKERRALFGAYRNPYTDDPSVAGMSRGLANTNVFWHGGKLLSSKEDSPPMMIDPDTLETIGEHTFDGALTSQTSTAHPKIDPRTGEMVFFGFAAKGETTPDIAYYEADSRGVVIHETWLRAPYSSMVHDFAVTQNFVVFPIIPLTSDLERLKAGGPHYAWDPHKDVYLGVLPRKGNGADIRWYRGSNRFASHIMGAHDDGRHIFIDTPVSESNFFPFFPDLSGAGYEAEKAKGYLSRWTIDTMGESNGFTETKIARYPGEFPRMDDRRETLPYNWGVLALSDVPEQESPGRGFRWISSIDLNTGHSQIYYPGHDCSVAEPLFVPAHDNAEEREGYVFVVIGRHEEMRSDLVILDAAHLDAPPVATLKMPLRIRSGLHGNWVSATELARERTA

SEQ ID NO: 37 - DNA, 1473 bp, synthetic construct
  source 1..1473: mol_type = other DNA
atgaacatccgcttcccgaataatgacttcaccagcttcgaagccccgatgcgcgccgaaattgatctgaaaggcctggaagttattcagggcgaagttccgcgtcagctgagcggcggcttcttccgcctgattggtgatcgcaaatggccggcattcgttgaaaatgatatcttcatgctgggtgaagatggcatggcaagttgcttctacttcaaagatggtcaggttgacttcaaaatgcgttatgtgcgcaccccgcgcttcgttgccgaagaaaaagccggccgtgcactgttcggtcattatcgcaatccgttcagcgatgatccgagtgttgcaggcctgagtcgcggtaccgccaatgtgaccgtgctggttcatgccggcaaaatgctggcactgaaagaagatagcccggccattgaactggacccgttcaccctggaaaccattggcgaatatgattggggtggtgttgtgaccgccaaagcaattaccgcacatccgaaaattgatccgaaaaccggcgaactggtgttcttcggttattgtgccgcaggtccggcaacccgcgatattgcatattatgaagcagatgccgaaggtcgcattattcatgaaacctggttcgaagccccttatacctgtatggttcatgatattatggttaccgaaaattacgttgtgttcccgattaccccgctgcgccatgaactggaatggattgaacgcaatgaaccggccttcaaatgggaccctagtgaagatgtgtatctgggtgttctgccgcgtaaaggtaaaggtgaacaggtgcgctggttcaaaggtccgaatcagtgccatggtcataccgtgggtgcattcgatgatggccgcttcatctatgcagataataccttcagtaaacgtagcttcttccacttcttcccgaataccgatggcagtgaattcaatccggcagatgccatgccgttcattaaacgcctggttattgatatggaaagtaatgatgatagcttcaccgaacagcagatgttcggttatccgtgcgaactgccgcgtattgatacccgcttcgaaaccatgccgtataacttcgttaccatgaatctgctggatgttccggaacaggaacgcattggtatgggcttccagtgggtggcaaccatggatattaatggccagaaaccggcaaaaatctattatgcaggtcatgattgtagcattggtgaaccgcagttcgtgccgcgtagcgatgatgccgcagaaggtgatggctatattctgaccgtggttggtaaacatcgtgaaatgcgcagtgaactgctgattctggatgcaggcgatattaccgcaccgccgattgcaaccgtggcactgccgttccgcctgcgcagtaccggtcatggctattggtatggtacccgtcagctgcgtggcgaaaccaatattattggcgcctggtaataa

SEQ ID NO: 38 - protein, 489 aa, Novosphingobium sp.
MNIRFPNNDFTSFEAPMRAEIDLKGLEVIQGEVPRQLSGGFFRLIGDRKWPAFVENDIFMLGEDGMASCFYFKDGQVDFKMRYVRTPRFVAEEKAGRALFGHYRNPFSDDPSVAGLSRGTANVTVLVHAGKMLALKEDSPAIELDPFTLETIGEYDWGGVVTAKAITAHPKIDPKTGELVFFGYCAAGPATRDIAYYEADAEGRIIHETWFEAPYTCMVHDIMVTENYVVFPITPLRHELEWIERNEPAFKWDPSEDVYLGVLPRKGKGEQVRWFKGPNQCHGHTVGAFDDGRFIYADNTFSKRSFFHFFPNTDGSEFNPADAMPFIKRLVIDMESNDDSFTEQQMFGYPCELPRIDTRFETMPYNFVTMNLLDVPEQERIGMGFQWVATMDINGQKPAKIYYAGHDCSIGEPQFVPRSDDAAEGDGYILTVVGKHREMRSELLILDAGDITAPPIATVALPFRLRSTGHGYWYGTRQLRGETNIIGAW

SEQ ID NO: 39 - DNA, 1449 bp, synthetic construct
  source 1..1449: mol_type = other DNA
atggtgtggttcccgccgccgaaagatccgttcgatgcaccgatgcgcgttgaagccgatattcgtggtgttcaggtgacccagggtgaagttccggaacatctggatggtacctattatcgttgtgttagcgatcgccagtggccgagttatgttgaaaatgatctggaactgttcaatagtgatggtatgattattagcttccgcttccataaaggccgtgttgacttcaaaagccgttatgtgcgcaccccgcgcttcgttgccgaagaaaaagccggtcgcgccctgttcggcgcctatcgtaatccgttcgatgatgatccgagtgtggcaggtattagccgtggcctggccaataccaatgtgttcttccatggtggtaaactgtatgccagcaaagaagatagtccgccgattctgattgatccggataccctggaaaccattggtgaatatgacttcgaaggtgcactgaccagtcgcaccgcaaccgcccatccgaaattcgatccggtgaccggcgaaatggtgttcttcggctatgaagcaaaaggtccgggcaccccggatattgcatattatgaagcagatccgagtggccgtattattcatgaagcatggctggaagccccgtataccagtatggttcatgattgggccgtgacccagaacttcgttatcttcccgattattccgctgcgcagcgatattgaatggattaaaaaaggtcagagttacttccagtgggacccttatgaagatgtgtatctgggcgttatgccgcgtaaaggtaccgccaaagatctgcgttggtatcgtggcagtaatcgcttcgcaagtcatattatgggtgcatacgatgatggccgttatgtgtatattgataccccggttggccagaccaattacttcccgtggttcccggatattaccggtgcaccgtatgatccggaacgcagtaaaggctatctgagccgctggaccattgatacccagggtgagagcgatggcttcaccgaaaccagactgagcgatctgccgggtgaattcccgcgcatggatgatcgtcaggaaaccttaccgtatcattggggcattatgctgctgaccgatgtgccgggtcgtaatgcaccgggtggtggcttccgttggctgatgggtattgatctgaaaaccggccgcaaacagatctattatccgggcgatcattgcaccctgggtgaagccatcttcgttccggccgcaccggatgccccgcctggtgtgggttatgttctgaccgttgtggcccgtcgtaaagaaatgcgcagtgatctggttctgctggatgcacagcgcctggatggtgaaccggtgtgtaccctgagcctgccgatgcgcattcgcatgggtgttcatggtaattgggttagcgccgccgaactggcaggtcgtaccgattaataa

SEQ ID NO: 40 - protein, 481 aa, Thermopolyspora flexuosa
MVWFPPPKDPFDAPMRVEADIRGVQVTQGEVPEHLDGTYYRCVSDRQWPSYVENDLELFNSDGMIISFRFHKGRVDFKSRYVRTPRFVAEEKAGRALFGAYRNPFDDDPSVAGISRGLANTNVFFHGGKLYASKEDSPPILIDPDTLETIGEYDFEGALTSRTATAHPKFDPVTGEMVFFGYEAKGPGTPDIAYYEADPSGRIIHEAWLEAPYTSMVHDWAVTQNFVIFPIIPLRSDIEWIKKGQSYFQWDPYEDVYLGVMPRKGTAKDLRWYRGSNRFASHIMGAYDDGRYVYIDTPVGQTNYFPWFPDITGAPYDPERSKGYLSRWTIDTQGESDGFTETRLSDLPGEFPRMDDRQETLPYHWGIMLLTDVPGRNAPGGGFRWLMGIDLKTGRKQIYYPGDHCTLGEAIFVPAAPDAPPGVGYVLTVVARRKEMRSDLVLLDAQRLDGEPVCTLSLPMRIRMGVHGNWVSAAELAGRTD

SEQ ID NO: 41 - DNA, 1446 bp, Amycolatopsis acidiphila
  source 1..1446: mol_type = genomic DNA
atgaccacgcgttttccccaggacccgatgttcgcggggttcagcgccccggtccgcgtcgaggccgacatctacgatctcgaggtcgctcagggcgaggtgcccgggcagctcgacggcacctactaccgggtggtgtgcgaccggcagtggccgccgatggtggcgggcgacatcccgttcaacgccgacggcatggtgatgtcgttccgcttcgagcgcggtcacgtcgacttcaagagccgttacgtgcggacgccgaggttcgaggccgagcggaaggcccggcggtcgttgttcggcgcgtaccgcaacccgttcacggacgacccatcggtcgccgggatgaaccggacgctggccaacaccaacgtgttctggcacggcgggaagctgctcgcgtcgaaggaggacagcccgccgatccagatcgaccccgatacgctggacaccgtcggggtgcacaccttcgacggggcgctgacctcgcagacctccaccgcgcatccgaagttcgatcccaggaccggcgagatggtgttcttcgggttcgccgcgaagggcgagtgcacccccgacatcgccttctacgaggcggaccgcgacgggaacctcgtgcacgagacctggttccaggccccgtactcgagcatggtgcacgacttcgcggtgacccagaacttcgtggtgttcccgatcatcccgctggtcagcgacctgggccggatccgcgcgggcctgtcgcactacgcgtgggacccgggcagggacgtccacctcggcgtgctgccgcgcaagggccgggtcgaggacctgcgctggtaccggggcggcacccggttcgccagccacatcctcggggcctacgacgacggcaggcacatccacatcgacaccccggtgtcggagtcgaactacttcccgttcttccccgacctgtcgggcgcacccgccgaccccgggaagaccaaggggtacctgtcgcggtggacgatcgacacgatgggcgaacggggcagcttcacccaggaacggctcaccacgcacgcgggcgagttcccgcggatggacgaccggttcgagacgctgccgtactcgtggggcgtcatgggcatgtacgaggtgccgggggagccgaggccgggccggggcttccggtgggtcggcgcggtcgacctcgagcacaggcgaacgacgacgcactacgtcggcgactgctcgtccgtcggcgagcccctcttcgtcccggcacacgagaaggccgggcacgcggagggctacgtgctggcggtcgtgggccggcacgacgagatgcgcagcgacctgctgatcctcgacgccgggcggatcgacgcgccgcccgtggcgacggtgaagctgccgatccgggtgccgtacgggctgcacggcaactgggtcacccgcggcgagctgcagcggggctga

SEQ ID NO: 42 - protein, 481 aa, Amycolatopsis acidiphila
MTTRFPQDPMFAGFSAPVRVEADIYDLEVAQGEVPGQLDGTYYRVVCDRQWPPMVAGDIPFNADGMVMSFRFERGHVDFKSRYVRTPRFEAERKARRSLFGAYRNPFTDDPSVAGMNRTLANTNVFWHGGKLLASKEDSPPIQIDPDTLDTVGVHTFDGALTSQTSTAHPKFDPRTGEMVFFGFAAKGECTPDIAFYEADRDGNLVHETWFQAPYSSMVHDFAVTQNFVVFPIIPLVSDLGRIRAGLSHYAWDPGRDVHLGVLPRKGRVEDLRWYRGGTRFASHILGAYDDGRHIHIDTPVSESNYFPFFPDLSGAPADPGKTKGYLSRWTIDTMGERGSFTQERLTTHAGEFPRMDDRFETLPYSWGVMGMYEVPGEPRPGRGFRWVGAVDLEHRRTTTHYVGDCSSVGEPLFVPAHEKAGHAEGYVLAVVGRHDEMRSDLLILDAGRIDAPPVATVKLPIRVPYGLHGNWVTRGELQRG

SEQ ID NO: 43 - DNA, 1506 bp, Rhodococcus sp.
  source 1..1506: mol_type = genomic DNA
atgattcacgccgcgtcactggacgaccgtgggtcaccggttcatccgaaggagacgcacgtgattcgctttcccgtcgccgacgatccgttcgacgcaccgatgcgtgtcgaggccgatatcaccgatctcgagtgcacccagggcgccgtgcccgaacacctcgacggcacctactaccgcgtggtagtcgacagacactatccctcgatggtcgacaacgatcttgcccttttcaactctgacggtatggcaatgtctttcaccttcagcaaggggcgggtggacttcaaaacccggtatgtgcgcacgccacggttcgtagccgaggaaaaggctggcaaatcactgttcggcgcctaccgtaatgcctcgaccgacgacccatcggtcgcagggatccgtcgtgggctggcgaacaccaatgtcttctttcacggtgggcgtctgtacgcctccaaggaggacagtccacccatacttctggaccccttgagcctcgagacactcggcgaatacgacttccaaggcgcactgacctccgaaacatctacagcacacccgaaagtcgatccacgcaccggagaaatggtgttcttcgggtacggagccaagggcgaagccacacctgacatcgcgtactacgaggccgacgcatccgggcagatcattcatgaggcctggctcactgctccctactcgtgcatgatccatgactttgcagttactcagaactacgtcatcttccctatcgtgccgctacgaagcagtacggagtggatcgcacagggaaacaaccagtacttctgggatccggatgaggatgtgtatctgggagtactcccacgtaaggggagcggaaaggaccttcgctggttccgtggcagcaatcgattcgccagtcacatcatgggtgccttcgacgacgggcgacacatctacatcgacacaccggtcgccgagacgagctactttccctggttccccgacatcagtggcgccgcctacgatcctgacaggagcaaaacacatctgtcacgctggacgatcgacctcgccgattccaccggcggtttcaccgagcggcgactgatgaccgccgccggcgaattcccccgaatggacgaccgtcaggaaaccctcgcttacgactggggggtactggcgatcaatgatctacccggcagctccaagcccggcccgggatttcgctggatcggtgccgtcaacctatcctccggctcgaccaagttctacgacccaggtccgaacgccactgtcggagaaccgatcttcgtaccgggccaggcggatgctaaaccgggccacgggtacgtcttcgtcgtcgttgctcgccgcgacctcatgcgcagcgacctggtcatactcgacgcgcagaatctcgacaaacctccggttgccaccctgtcgctaccaatacggatcaagtcgggtcttcacggtaactgggtcggggcagacgaactggctggcaggaccgcgtga

SEQ ID NO: 44 - protein, 501 aa, Rhodococcus sp.
MIHAASLDDRGSPVHPKETHVIRFPVADDPFDAPMRVEADITDLECTQGAVPEHLDGTYYRVVVDRHYPSMVDNDLALFNSDGMAMSFTFSKGRVDFKTRYVRTPRFVAEEKAGKSLFGAYRNASTDDPSVAGIRRGLANTNVFFHGGRLYASKEDSPPILLDPLSLETLGEYDFQGALTSETSTAHPKVDPRTGEMVFFGYGAKGEATPDIAYYEADASGQIIHEAWLTAPYSCMIHDFAVTQNYVIFPIVPLRSSTEWIAQGNNQYFWDPDEDVYLGVLPRKGSGKDLRWFRGSNRFASHIMGAFDDGRHIYIDTPVAETSYFPWFPDISGAAYDPDRSKTHLSRWTIDLADSTGGFTERRLMTAAGEFPRMDDRQETLAYDWGVLAINDLPGSSKPGPGFRWIGAVNLSSGSTKFYDPGPNATVGEPIFVPGQADAKPGHGYVFVVVARRDLMRSDLVILDAQNLDKPPVATLSLPIRIKSGLHGNWVGADELAGRTA

SEQ ID NO: 45 - DNA, 1422 bp, Amycolatopsis sacchari
  source 1..1422: mol_type = genomic DNA
atgaacttcaacgcgccgatgcgggtcgaggccgacatctacgacctcgaagtggcacaaggcgaagtacccgaacaactcgacggcacgtactaccgcgtggtgtgcgaccggcagtacccgtcgttcgtgccgaatgacatccccttcaatgccgacggcatggtcacgtcgttccgcttctcgaagggacacgtcgacttcaaaagccgttacgtgcgcacgccgcggttccaggcggagcggagcgcacgccgcgcgttgttcggcgcgtaccgcaacgcctacaccgacgacccgtccgtcgcggggatgaaccggaccttggccaacaccaacgtgttctggcacggcgggcgactactggcgtccaaagaggacagtccgccgatcgagatcgacccgctgacgctggacacggtgggcgtccacaccttcgacggcgcactcacgtcgcagacctccaccgcgcacccgaaattcgacccgcgcacgggcgaaatggtgttcttcggttacgcggcgaaaggtgaaggcacgccggacatcgtgtactacgaggccgaccccgccgggaacctcgtgcacgaaacctggttccaggccccgtatccgagcatggtgcacgacttcgcggtgacgcagaacttcgtcgtgttcccgatcatcccgctcgtgtcggacctgggcaggctgcggcagggcctgccgttgttcgcgtgggacccggaaaaggacgtgttcctcggcgtgctgccgcgcaagggcggcgtggaggacctgcgctggtaccgcggcagcacgcggttcgccagccacatcctcggtgcttacgacgacgggcggcacatccacgtcgacaccccggtttcggagacgaactacttcccgttcttcccggatctgtcgggcgcgcccgtggaccccgagaagaccaagggctacctgtcccgctggacgatggacaccctgggcgactcggcgacgttcaccgaggaacggctgaccgcccatgccggggagttcccgcgcatggacgaccggttcgagacgctgccctactcctggggcgtgctgggcctgtacgaggtgccgggcgagccgcggccgggcggcgggttccggtggatcggcgccgtggacctcgcccgcaagcgcacgacgtcccactacgtcggtgacgcgtcgtcggtcggcgagccgctgttcgtgcccgcgcacgcggacgccgcacagggcaagggttacgtgctggccgtggtgggccgccacgacgagatgcgcagcgacctgctgatcctcgacgcgcagcggatcgacgccccaccggtggcgacggtgaagctgccgatccgcgtgccctacggcctccacggcaactgggtgacgacagccgaactccaggcggcggggaaggtctag

SEQ ID NO: 46 - protein, 473 aa, Amycolatopsis sacchari
MNFNAPMRVEADIYDLEVAQGEVPEQLDGTYYRVVCDRQYPSFVPNDIPFNADGMVTSFRFSKGHVDFKSRYVRTPRFQAERSARRALFGAYRNAYTDDPSVAGMNRTLANTNVFWHGGRLLASKEDSPPIEIDPLTLDTVGVHTFDGALTSQTSTAHPKFDPRTGEMVFFGYAAKGEGTPDIVYYEADPAGNLVHETWFQAPYPSMVHDFAVTQNFVVFPIIPLVSDLGRLRQGLPLFAWDPEKDVFLGVLPRKGGVEDLRWYRGSTRFASHILGAYDDGRHIHVDTPVSETNYFPFFPDLSGAPVDPEKTKGYLSRWTMDTLGDSATFTEERLTAHAGEFPRMDDRFETLPYSWGVLGLYEVPGEPRPGGGFRWIGAVDLARKRTTSHYVGDASSVGEPLFVPAHADAAQGKGYVLAVVGRHDEMRSDLLILDAQRIDAPPVATVKLPIRVPYGLHGNWVTTAELQAAGKV

SEQ ID NO: 47 - DNA, 1473 bp, Streptomyces sp.
  source 1..1473: mol_type = genomic DNA
atgcagcagatcaggtttccgcagaacgaattccagagtttcgaggcgcccatgcgcgcggaggtcgacatccgaggactggaagtagttcagggcgaggttccggagcatctcgacggcggcttctaccgactgctcggcgatcgccagtggccttccttcatcgaaaacgacatcttcctgctgaacgaagatggcatggcttcatgcctgcgcatctcgaacggccgcgtggacttccggacacgatatgtcaaaactccgcgattcgttgccgaggagcaggccggccgggcacttttcggcgcgtaccgcaacccgctcaccgacgacccgtcagtgcagggcgtccgccgcggaacagccaacgtgaccatgctgtcgcacgccggcaagttcttcgccctcaaggaggacagcccgccggtcgagatggatcccttcaccctggagacgatcggggagtacaccttcggcggagacttcacatccgaggccttcaccgcccatccgaagatcgacccgcgcaccggcgaactggtgttctacggctactgcgcgaaaggcccggccacgaaagatatcgcatactacgaggcggaccagaccgggaagatcatccacgagacctggttcgaaacgccatacacctgcatggtgcacgacatcatggtgaccgaaaactacgtggtgttccctattacgccgctgcgtcacgaatacgaatggctggagaagcgcgagccggcgtttaagtacgacaccagcgagcaggtgcaccttggtgtactgccgcgcaagggcaccgcagatcagattcgctggttcgtcggccccaaccagtgtcacgggcacaccgtgggcgcctatgacgacggtcgctacatctacgccgacaacacgctgagtgtccgtagcttcttcccgttcttccccaacgccgacggttcgcccttcgacccggaagacacgaaaccctacatgaagcgctgggtcatcgacatgggcagcgagggcgacactttcacggagcgtcagctgctgcagtatccctgcgaactcccgcgcatcgatacccgtttcgagacccagccatacagcttctgtgcgctgaaccttctcgatgtgcccggtcaggatcgggtcggcggcgggtggcagtggatcgcgaccatggatgtcaccggaaagaagccgacccggattcgctatgctggtgacaactgctcgatcagcgagccccagttcgttccggcgcacgacggggctgccgaaggcgaaggctacgtcctcgttgtcgtcggacgacacgacgaaatgcgcagcgagttcctgattctcgacgcgcagaatatcgacggcccgccgatcgcgaccgtcgccctgccgatgcgcgtgcgcagcacgggccacggattttggtacgggaagcgtcaactgatgggcgagacgcatctcctcggggcgaactga

SEQ ID NO: 48 - protein, 490 aa, Streptomyces sp.
MQQIRFPQNEFQSFEAPMRAEVDIRGLEVVQGEVPEHLDGGFYRLLGDRQWPSFIENDIFLLNEDGMASCLRISNGRVDFRTRYVKTPRFVAEEQAGRALFGAYRNPLTDDPSVQGVRRGTANVTMLSHAGKFFALKEDSPPVEMDPFTLETIGEYTFGGDFTSEAFTAHPKIDPRTGELVFYGYCAKGPATKDIAYYEADQTGKIIHETWFETPYTCMVHDIMVTENYVVFPITPLRHEYEWLEKREPAFKYDTSEQVHLGVLPRKGTADQIRWFVGPNQCHGHTVGAYDDGRYIYADNTLSVRSFFPFFPNADGSPFDPEDTKPYMKRWVIDMGSEGDTFTERQLLQYPCELPRIDTRFETQPYSFCALNLLDVPGQDRVGGGWQWIATMDVTGKKPTRIRYAGDNCSISEPQFVPAHDGAAEGEGYVLVVVGRHDEMRSEFLILDAQNIDGPPIATVALPMRVRSTGHGFWYGKRQLMGETHLLGAN

SEQ ID NO: 49 - DNA, 1455 bp, Pseudonocardia cypriaca
  source 1..1455: mol_type = genomic DNA
atgaccaggttcccgcagcgggacggcgcgagcgcgttcgaggcgccgatgcgcgtggaggccgacatcgcaggcctcgaggtggtgcagggcgaggtgcccgagcacctgtccggcacctactaccgcgtggtcaccgaccggcagtggccaccggccgtcctccccgacgtaccgacgttcaacgacgacggcatggccatgtacttccgcttccacggcggccgggtcgacttccgcagccgctacgtgcgcacaccgcgctacgaggccgaggcgaaggcgggccgggcgctcttcggcgcctaccgcaacccgctgaccgacgacccgtcggtcgccggcatctctcgcgggctgggcaacacgaacgtcttctaccacggcgggaggctgtacgcgtccaaagaggacagcgctccgatcctgctcgacccgatgacgctcgagacgatcggcgagtacaccttcgagggcacgctcacgtcgcagacgtgcaccgcgcaccccaagatggatccgcgcaccggcgagatggtgttcttcggctacgccgccaagggcgagaccacgccggacatcgcctactacgaggccgacccggacggccggatcgtccacgagacgtgggtccgcgcgccgtactcgagcatggtccacgactgggccgtcacgcagaacttcgtggtcttcccgatcatcccgatgcgcagcgagtacgcctggctccaggagcggcagccgcacttccagtgggacccggaccgggaggtgtacctcggtgtgctgccgcgcaagggcggcgccgaccaggtgcgctggctgcgcgggtccaaccggttcgcgagccacatcctcggcgcgtgggacgacgggcgccgcatccacatcgagaccccggtcgggcagagcaacttcttcccgttcttcccggacgtgacgggcgctccgtacgaccccgtgcgcgcccgcggccacctgtcccgctggacggtcgacatggggtccgacagcgggaccttcaccgaggaacggctcaccgacgtcgcaggcgagttcccgcgcatggacgaccgcttcgagacgttgccctaccggcgcgggttcctggcgctgaaccacgtgcccggcgatccgcggccgggcgccggcttccgctggatcggcgcgttcgacaccaccggtgccgcgccgccgcaggtcttctaccccggcgacgactgctcggtggccgagccgctcttcgtccccgggaacgacgacgcgccagagggccacggctacgtgttcgtggtggtcgggcggcaccaggagatgcgcagcgacctggtgatcctcgacgcccagcggctcgacggcccgcccgtcgccacggtggcgcttcccatccgcgtccgccgcggcctgcacggcaactgggtgagcagccgcgagctcgccgcccgtaccgcctga

SEQ ID NO: 50 - protein, 484 aa, Pseudonocardia cypriaca
MTRFPQRDGASAFEAPMRVEADIAGLEVVQGEVPEHLSGTYYRVVTDRQWPPAVLPDVPTFNDDGMAMYFRFHGGRVDFRSRYVRTPRYEAEAKAGRALFGAYRNPLTDDPSVAGISRGLGNTNVFYHGGRLYASKEDSAPILLDPMTLETIGEYTFEGTLTSQTCTAHPKMDPRTGEMVFFGYAAKGETTPDIAYYEADPDGRIVHETWVRAPYSSMVHDWAVTQNFVVFPIIPMRSEYAWLQERQPHFQWDPDREVYLGVLPRKGGADQVRWLRGSNRFASHILGAWDDGRRIHIETPVGQSNFFPFFPDVTGAPYDPVRARGHLSRWTVDMGSDSGTFTEERLTDVAGEFPRMDDRFETLPYRRGFLALNHVPGDPRPGAGFRWIGAFDTTGAAPPQVFYPGDDCSVAEPLFVPGNDDAPEGHGYVFVVVGRHQEMRSDLVILDAQRLDGPPVATVALPIRVRRGLHGNWVSSRELAARTA

SEQ ID NO: 51 - DNA, 1638 bp, Salinibacterium hongtaonis
  source 1..1638: mol_type = genomic DNA
atgcggacgcaggccgtggccacgacggagctggaccgacaggtccggaccgagggcgagacccgccacgattcactcgacggagagcacatgactcatcagaataatccagaagaagacgcgcgcgccgaggagcttgcgtgggtgcggacccaaccaaaggttcctatcgtcaccccgtctccctatccgatgatccgcttcgccgagcccggacatccttttgaccgtcccgtgcgcgtggaagcagacattgaggggctcgaatgcgtccagggcgagatcccggcgcacctcaacggcacctactacaaggtcgtgagcgaccgtcagttcccttcctttgtggagaatgatctgggctacttcaacgacgacggcatggcactgtctttccgcttccacgacggcaaggtggactacaagagccggttcatccgcaccccgaggttcgaggcggagaaggcggccggacgctcgctgttcggcaagtaccgcaaccagttcgacgacgacgcaatggtcgagggcatgctgcgaggcaacgccaatacgaacctcttctttcacggcggcaagctgtacgccgccaaggaggactctccccccatcatcctcgaccccatcacgctggagacgctcggggtctacgacttcgagggttcgctgacctccaaatcttcaacggcgcatcccaagatcgaccccatcaccggcgagatggtgttcttcggctatgcggccaagggcgacacgacgcgggacattgcctactacgaggccgacagcacgggtcagatcatccacgagacgtggttcaaggcgccgtattcggccatggtgcatgacttcgccgtgactcagaactacgtggtgttccccatcatccctctgcgcagcgagcgcgagtggatcgagaagggcgaatcgtccttccagtgggatgcctcggaggatgtctacctcggcgtcatgccgcgcaagggcgacggagcgaagatccggtggttccgtgggcgctcgcgcttcgcaagccacaccctgggcgcctacgacgacggccgttacatctacatcgacagtccggtgtcggagagcaagtactttccttggttcccgaaccgcgatgggtcgccgcacgacgcggtcaagggcaggaccacgctgacacggtggacgatcgatcttcacgacggctcggatgggttcaccgagagcagcatctcggatctcggtgtcgagttccctcgcatggatgatcgtttcgagacgctgcagcacacctggggggcgatggccgtcagttccgtgccggggatcgagatgcagggccatggtttccgctgggtgtcgacagtggactatgcaactggcgcccagaagctctattacccaggcgagggctcgaccgtgggtgagcccgtgttcgtgccgggggctcctggtgcgcccgaggggcacggctacgtgctcgtcctggtgtcacgccgcgacgccatgcacagcgacctcgtggtgctcgacgctcagaacgtcgataaggagccggtctgcacgctgcgcatgccccttcgcgtgccgaagggtctccacggtaactgggtgtccgacgaggagctcggacagcgcaccgactga

SEQ ID NO: 52 - protein, 545 aa, Salinibacterium hongtaonis
MRTQAVATTELDRQVRTEGETRHDSLDGEHMTHQNNPEEDARAEELAWVRTQPKVPIVTPSPYPMIRFAEPGHPFDRPVRVEADIEGLECVQGEIPAHLNGTYYKVVSDRQFPSFVENDLGYFNDDGMALSFRFHDGKVDYKSRFIRTPRFEAEKAAGRSLFGKYRNQFDDDAMVEGMLRGNANTNLFFHGGKLYAAKEDSPPIILDPITLETLGVYDFEGSLTSKSSTAHPKIDPITGEMVFFGYAAKGDTTRDIAYYEADSTGQIIHETWFKAPYSAMVHDFAVTQNYVVFPIIPLRSEREWIEKGESSFQWDASEDVYLGVMPRKGDGAKIRWFRGRSRFASHTLGAYDDGRYIYIDSPVSESKYFPWFPNRDGSPHDAVKGRTTLTRWTIDLHDGSDGFTESSISDLGVEFPRMDDRFETLQHTWGAMAVSSVPGIEMQGHGFRWVSTVDYATGAQKLYYPGEGSTVGEPVFVPGAPGAPEGHGYVLVLVSRRDAMHSDLVVLDAQNVDKEPVCTLRMPLRVPKGLHGNWVSDEELGQRTD

SEQ ID NO: 53 - DNA, 1473 bp, Streptomyces scabiei
  source 1..1473: mol_type = genomic DNA
atgcagcagatcaggtttccgcagaacgagttccagagcttcgaggcgcccatgcgcgcggaggtcgacatccgaggactggaagtagttcagggcgaggttccggagcatctcgacggcggcttctaccgactgctcggcgatcgccagtggccttccttcatcgaaaacgacatcttcctgctgaacgaagatggcatggcctcatgcctgcgcatctcgaacggccgcgtggacttccggacacggtatgtcaaaactccgcgattcgttgccgaggagcaggccggccgggcacttttcggcgcgtaccgcaacccgctcaccgacgacccgtcagtgcaaggcgtccgccgcggaacagccaacgtgaccatgctctcgcacgccggcaagttcttcgccctcaaggaggacagcccgccggtcgagatggatcccttcaccctggagacgatcggggagtacaccttcggcggagacttcacgtccgaagccttcaccgcccatccgaagatcgacccgcgcaccggcgaactggtgttctacggctactgcgcgaagggcccggccacgaaagatatcgcatactacgaggcggaccggaccgggaagatcatccacgagacctggttcgaagcaccatacacctgcatggtgcacgacatcatggtgaccgaaaactacgtggtgttccctattacgccgctgcgtcacgaatacgaatggctggagaagcgcgagccggcgttcaagtacgacaccggcgagcaggtgcaccttggtgtactgccgcgcaagggcaccgcagatcagattcgctggttcgtcggccccaaccagtgtcacgggcacaccgtgggcgcctatgacgacggtcgctacatctacgccgacaacacgctgagtgtccgcagcttcttcccgttcttccccaacgccgacggttcgccgttcgacccggaagacacgaaaccctacatgaagcgctgggtcatcgacatgggtagcgagggcgacactttcacggagcgtcagctgctgcagtatccctgcgaactcccgcgcatcgatacccgtttcgagacccagccctacagcttctgtgcgctgaaccttctcgatgtgcccggtcaggatcgggtcggcggcgggtggcagtggatcgcgaccatggatgtcaccggaaagaagccgacccggattcgctatgctggcgacaactgctcgatcagcgagccccagttcgttccggcgcacgacggggctgccgaaggcgaaggctatgtcctcgttgtcgtcggacgacacgaggaaatgcgcagcgagttcctgatcctcgacgcgcagaatatcgacggcccgccgatcgcgaccgtcgcgttgccgatgcgcgtgcgcagcacgggccacggattttggtatgggaagcgtcaactgatgggcgagacgcatcttctcggggcgaactga

SEQ ID NO: 54 - protein, 490 aa, Streptomyces scabiei
MQQIRFPQNEFQSFEAPMRAEVDIRGLEVVQGEVPEHLDGGFYRLLGDRQWPSFIENDIFLLNEDGMASCLRISNGRVDFRTRYVKTPRFVAEEQAGRALFGAYRNPLTDDPSVQGVRRGTANVTMLSHAGKFFALKEDSPPVEMDPFTLETIGEYTFGGDFTSEAFTAHPKIDPRTGELVFYGYCAKGPATKDIAYYEADRTGKIIHETWFEAPYTCMVHDIMVTENYVVFPITPLRHEYEWLEKREPAFKYDTGEQVHLGVLPRKGTADQIRWFVGPNQCHGHTVGAYDDGRYIYADNTLSVRSFFPFFPNADGSPFDPEDTKPYMKRWVIDMGSEGDTFTERQLLQYPCELPRIDTRFETQPYSFCALNLLDVPGQDRVGGGWQWIATMDVTGKKPTRIRYAGDNCSISEPQFVPAHDGAAEGEGYVLVVVGRHEEMRSEFLILDAQNIDGPPIATVALPMRVRSTGHGFWYGKRQLMGETHLLGAN

SEQ ID NO: 55 - DNA, 1470 bp, Trebonia kvetii
  source 1..1470: mol_type = genomic DNA
atgacggtgcggttccctgacgagcctggcttccagggccggttcgctccggtacggcttgaaggcgagatccgcggtgtcgaggtgacgcagggggagatccccgcgtcgctgagcggcacgttctaccggggcggcgcggaccctgcctggccgccgttcgtcgagcgcgacttctacttcaacgccgacggcatggtcgcgatgttccggttcgccaacgggtactgcgacttcaggtccaggtatgtgcgcacgccccgcttcgtggcggagcgtgcggcgcggcggtcgctgttcggcgcctaccgcaacccgttcaccgacgacccgtcagtcgcgggcctgtcccgcgggctcgccaacaccaacgtgtactggcacggcgggcggctgctcgcctccaaggaggacagcgcgccggtccagatcgacccggacacgctggacacggtcggcgagttcacctgggagggcgacctgtcctcgcagaccgcgaccgcgcacccgaagatcgacccgcgggacggttcgctggtgttcttcggctacatggccaagggcgaggcgacgcgggacatcgcctactacgaggcggacgcggccggccggatcgtgcacgaggcctggttcgaggcgccgtactcctcgatggtgcacgactgggcggtaacagaaaacttcgtgatcttcccgatcatcccgctcaccgcctcgctcgaacggctggcggccggcggtccgctgtacgtgtgggacggatccgaggacgtctacctaggcgtcgtgccgcgccgtggtaacagcgtgcgctggtaccgcggcagcaaccggttcgcctcgcacatcatgaacgcctacgacgacggccggcacatccacatcgacacgccggtcggcgagaagtccgcgttcccctggttcccggacatcgcgggcgcgccgttcgacccggagaaggcgagcgcgcagctgacccggtggacgatcgacaccgccgcgccggaagacctcggtgacggtgccacggcgttcgaacagcggcggctcaccggctgctcgggcgagttcccgcgcaccgacgaccggtgggcgacccgcggctaccggttcggcgtcatcaatctcaccgacgtgccgggcgaacggccagacgacgggctgccgggattccgctggctcggccagatcgacccggcgaccggggcgatgaagaccaggttcgccgggcggaactccacggtgcaggaggcgatcttcgtaccgcctggcagggacaccgccaacggcgagggatacgtgatgcagctggtcgaccggcacgagaccggcacgaccgacctgctcatcctcgacgcgcagcgcatcgatgagccgccggtcgccacgctgcggatcccgatcaggatgcctggcggcctgcacggcaactgggtgacagacgagcagctcgcgcagcgagccggctag

SEQ ID NO: 56 - protein, 489 aa, Trebonia kvetii
MTVRFPDEPGFQGRFAPVRLEGEIRGVEVTQGEIPASLSGTFYRGGADPAWPPFVERDFYFNADGMVAMFRFANGYCDFRSRYVRTPRFVAERAARRSLFGAYRNPFTDDPSVAGLSRGLANTNVYWHGGRLLASKEDSAPVQIDPDTLDTVGEFTWEGDLSSQTATAHPKIDPRDGSLVFFGYMAKGEATRDIAYYEADAAGRIVHEAWFEAPYSSMVHDWAVTENFVIFPIIPLTASLERLAAGGPLYVWDGSEDVYLGVVPRRGNSVRWYRGSNRFASHIMNAYDDGRHIHIDTPVGEKSAFPWFPDIAGAPFDPEKASAQLTRWTIDTAAPEDLGDGATAFEQRRLTGCSGEFPRTDDRWATRGYRFGVINLTDVPGERPDDGLPGFRWLGQIDPATGAMKTRFAGRNSTVQEAIFVPPGRDTANGEGYVMQLVDRHETGTTDLLILDAQRIDEPPVATLRIPIRMPGGLHGNWVTDEQLAQRAG

SEQ ID NO: 57 - DNA, 1446 bp, Rhodococcus jostii
  source 1..1446: mol_type = genomic DNA
gtggaccgcttccccatccccgaagatccgttcgatgcgccgatgcgcctggaggccgacatcaccgacctcgaatgcgtccagggcgaagtgccggcgcacctcgacggcacctactaccgcgtcgtggtcgaccgccactacccctcgttcgtcgacaacgatctcgcattgttcaactccgacggcatggcaatgtccttcaccttccacaagggacgggtcgacttcaagagcagatatgtacgtaccccccgcttcgttgctgaggagaaggctggcaagtcgctgttcggcgcctaccacaacgcgctcaccgacgacccgtctgtggccggagttcgacgcggcctcgcgaacaccaacatcttctttcatggcggccgcctgtatgcctccaaggaggacagtccgcccatcctgctcgacccggtgaccctcgagaccctcggtgaatacgactttcaaggcgcgctcacctcggaaacctccacggcgcacccgaagatcgacccccgcaccggcgaaatggtgttcttcggctacgccgccaagggagaggcgacacccgacatcgcatactacgaggctgatccgaacggtcagatcatccatgagacctggatccgggcgccctactcgtgcatgatccacgacttcgcggtaactcagaactacgtcgtcttccccatcgttccactccgcagcagcatcgaatggctcgagaagggcaacccccaattcttttgggatccaaacgaagacgtgcacctaggcgttcttccacgcaaggggacagacaaggacctgcgctggttccgtggctcgaatcgattcgcaagccacatcatgggcgcctacgacgacggccgctacctacacctcgactcaccggtcgccgaaacgaactacttcccgtggttccccgacctgtccggcgctccctacaacccggaccggagcaagacccgcctgacgcgatggacgatcgacctgtcgggggagtcggagaccttcaccgaacgccgactgatggacgccgcaggcgaattccctcgcatggacgatcggcaagaaaccctggactacaactggggagtcatggcgatcaacgaccatcccgaacagcacaagacgggcggcggcttccggtgggtcggcgccgtcgacttgtccagcggaagaaccaagtactacgacccgggcgagaactccaccgtcggcgagccgatcttcgttcccggcaacgacaccgccggccccggcgccggctacgtattcgtcgtggtggcgcggcgcgaccacatgcacagcgaattggtcatcctcgacgcacaacacatcgatcgtcctcccgtcgccacgctgaaactgcccatgcgactcaaatccgggctgcacggcaactgggtcggggccgacgagatcgccggccgaaccgcctga

SEQ ID NO: 58 - protein, 481 aa, Rhodococcus jostii
MDRFPIPEDPFDAPMRLEADITDLECVQGEVPAHLDGTYYRVVVDRHYPSFVDNDLALFNSDGMAMSFTFHKGRVDFKSRYVRTPRFVAEEKAGKSLFGAYHNALTDDPSVAGVRRGLANTNIFFHGGRLYASKEDSPPILLDPVTLETLGEYDFQGALTSETSTAHPKIDPRTGEMVFFGYAAKGEATPDIAYYEADPNGQIIHETWIRAPYSCMIHDFAVTQNYVVFPIVPLRSSIEWLEKGNPQFFWDPNEDVHLGVLPRKGTDKDLRWFRGSNRFASHIMGAYDDGRYLHLDSPVAETNYFPWFPDLSGAPYNPDRSKTRLTRWTIDLSGESETFTERRLMDAAGEFPRMDDRQETLDYNWGVMAINDHPEQHKTGGGFRWVGAVDLSSGRTKYYDPGENSTVGEPIFVPGNDTAGPGAGYVFVVVARRDHMHSELVILDAQHIDRPPVATLKLPMRLKSGLHGNWVGADEIAGRTA

SEQ ID NO: 59 - DNA, 1473 bp, Streptomyces sp.
  source 1..1473: mol_type = genomic DNA
atgcagcagatcaggtttccgcagaacgagttccagagcttcgaggcgcctatgcgcgcggagatcgacatccgaggactggaagtagttcagggcgaggttccggagcatctcgacggcggcttctaccgactgctcggcgatcgccagtggccttccttcatcgaagacgacatcttcctgctgaacgaagatggcatggcctcatgcctgcgcatctcgaacggccgcgtggacttccggacacggtatgtcaaaactccgcgattcgttgccgaggaacaggccggccgggcacttttcggcgcgtaccgcaacccgctcaccgacgacccgtcagtgcaaggcgtccgccgcggaacagccaacgtgaccatgctctcgcacgccggcaagttcttcgccctcaaggaggacagcccgccggtcgagatggatcccttcaccctggagacgatcggggagtacaacttcggcggagacttcacgtctgaagccttcaccgcccatccgaagatcgacccgcgcaccggcgaactggtgttctacggctactgcgcgaaaggcccggccacgaaagatatcgcatactacgaggcggaccggaccgggaagatcatccacgagacctggttcgaagcaccatacacctgcatggtgcacgacatcatggtgaccgaaaactacgtggtgttccctattacgccgctgcgtcacgaatacgaatggctggagaagcgcgagccggcgttcaagtacgacaccggcgagcaggtgcaccttggtgtactgccgcgcaagggcaccgcagatcagattcgctggttcgtcggccccaaccagtgtcacgggcacaccgtgggcgcctatgacgacggtcgctacatctacgccgacaacacgctgagtgtccgcagcttcttcccgttcttccccaacgccgacggttcgccgttcgacccggaagacacgaaaccctacatgaagcgctgggtcatcgacatgggtagcgagggcgacactttcacggagcgtcagctgctgcagtatccctgcgaactcccgcgcatcgacacccgtttcgagacccagccctacagcttctgtgcgctgaaccttctcgatgtgcccggtcaggatcgggtcggcggcgggtggcagtggatcgcgaccatggatgtcaccggaaagaagccgacccggattcgctatgctggcgacaactgctcgatcagcgagccccagttcgttccggcgcacgacggggctgcggaaggcgaaggctatgtcctcgttgtcgtcggacgacacgaggaaatgcgcagcgagttcctgatcctcgacgcgcagaatatcgacggcccgccgatcgcgaccgtcgccctaccgatgcgcgtgcgcagcacgggccacggattttggtacgggaagcgtcaactgatgggcgagacgcatcttctcggggcgaactga

SEQ ID NO: 60 - protein, 490 aa, Streptomyces sp.
MQQIRFPQNEFQSFEAPMRAEIDIRGLEVVQGEVPEHLDGGFYRLLGDRQWPSFIEDDIFLLNEDGMASCLRISNGRVDFRTRYVKTPRFVAEEQAGRALFGAYRNPLTDDPSVQGVRRGTANVTMLSHAGKFFALKEDSPPVEMDPFTLETIGEYNFGGDFTSEAFTAHPKIDPRTGELVFYGYCAKGPATKDIAYYEADRTGKIIHETWFEAPYTCMVHDIMVTENYVVFPITPLRHEYEWLEKREPAFKYDTGEQVHLGVLPRKGTADQIRWFVGPNQCHGHTVGAYDDGRYIYADNTLSVRSFFPFFPNADGSPFDPEDTKPYMKRWVIDMGSEGDTFTERQLLQYPCELPRIDTRFETQPYSFCALNLLDVPGQDRVGGGWQWIATMDVTGKKPTRIRYAGDNCSISEPQFVPAHDGAAEGEGYVLVVVGRHEEMRSEFLILDAQNIDGPPIATVALPMRVRSTGHGFWYGKRQLMGETHLLGAN

SEQ ID NO: 61 - DNA, 1473 bp, Streptomyces griseorubiginosus
  source 1..1473: mol_type = genomic DNA
atgcagcagatcaggtttccgcagaacgagttccagagtttcgaggcgcccatgcgcgcggaggtcgacatccgaggactggaagtagttcagggcaaggttccggagcatctcgacggcggcttctaccgactgctcggcgatcgccagtggccttccttcaccgaaaacgacatcttcctgctgaacgaagatggcatggcgtcatgcctgcgcatctcgaacggccgcgtggacttccggacacggtatgtcagaactccgcgattcgttgccgaggagcaggccggccgggcacttttcggcgcgtaccgcaacccgttcaccgacgacccgtcagtgcaaggcgtccgccgcggaacagccaacgtgaccatgctctcgcacgccggcaagttcttcgccctcaaggaggacagcccgccggtcgagatggatcccttcaccctggagacgatcggggagtacaccttcggcggagacttcacgtccgaagcctgcaccgcccatccgaagatcgacccgcgcaccggcgaactggtgttcttcggctactgcgccaaaggcccggccacgaaagatatcgcatactacgaggcggaccgcaacgggaagatcatccacgagacctggttcgaagcaccgtacacctgcatggtgcacgacatcatggtgacccaaaactacgtggtgttccctatcacgccgctgcgtcacgaatacgaatggctggagaagcgcgagccggcgttcaagtacgacaccagcgagcaggtgcaccttggtgtactgccgcgcaagggcaccgcagatcagattcgctggttcgtcggccccaaccagtgtcacgggcacaccgtgggcgcctatgacgacagtcgctacatctacgccgacaacacgctgagtgtccgcagcttcttcccgttcttccccaacgccgacggttcgccgttcgacccggaagacacgaaaccctacatgaagcgctgggtcatcgacatgggtagcgagggcgacactttcacggagcgtcagctgctgcagtatccctgcgaactcccgcgcatcgatacccgtttcgagacccagccctacagcttctgtgcactgaaccttcttgatgtgcccggtcagaatcgagtcggcggcgggtggcagtggatcgcgaccatggatgtcaccggaaagaagccgacccggattcgctacgctggcgacaactgctcgatcagcgagccccagttcgttccggcgcacgacggggctgccgaaggtgaaggctatgtcctcgttgtcgtcggacgacacgacgaaatgcgcagcgagttcctgatcctcgacgcgcagaatatcgacggcccgccgattgcgaccgtcgccctgccgatgcgcgtgcgcagcacgggccacggattctggtacgggaagcgtcaactgatgggcgagacgcatcttctcggggcgaactga

SEQ ID NO: 62 - protein, 490 aa, Streptomyces griseorubiginosus
MQQIRFPQNEFQSFEAPMRAEVDIRGLEVVQGKVPEHLDGGFYRLLGDRQWPSFTENDIFLLNEDGMASCLRISNGRVDFRTRYVRTPRFVAEEQAGRALFGAYRNPFTDDPSVQGVRRGTANVTMLSHAGKFFALKEDSPPVEMDPFTLETIGEYTFGGDFTSEACTAHPKIDPRTGELVFFGYCAKGPATKDIAYYEADRNGKIIHETWFEAPYTCMVHDIMVTQNYVVFPITPLRHEYEWLEKREPAFKYDTSEQVHLGVLPRKGTADQIRWFVGPNQCHGHTVGAYDDSRYIYADNTLSVRSFFPFFPNADGSPFDPEDTKPYMKRWVIDMGSEGDTFTERQLLQYPCELPRIDTRFETQPYSFCALNLLDVPGQNRVGGGWQWIATMDVTGKKPTRIRYAGDNCSISEPQFVPAHDGAAEGEGYVLVVVGRHDEMRSEFLILDAQNIDGPPIATVALPMRVRSTGHGFWYGKRQLMGETHLLGAN

SEQ ID NO: 63 - DNA, 1449 bp, synthetic construct
  source 1..1449: mol_type = other DNA
atggttcgcttcccgccgccgaaagatccgttcgatgcaccgatgcgtgtggaagcagatattcgcggtgtgcaggttacccagggcgaagttccggaacatctggatggtatctattatcgttgcgttagtgatcgtcagtggccgagctatgtggaaaatgatctggaactgttcaatagcgatggtatgattattagcttccgcttccataaaggccgtgtggacttcaaaagtcgttatgtgcgtaccccgcgcttcgtggccgaagaaaaagccggtcgtgcactgttcggtgcctatcgtaatccgttcgatgatgatccgagcgtggcaggcattagtcgcggcctggcaaataccaatgtgttctatcatggtggcaaactgtatgcaagtaaagaagatagtccgccgattctgctggaccctattaccctggaaaccattggcgaatatgacttcgaaggtgcactgaccagtcgcaccagtaccgcccatccgaaattcgatccgattaccggtgaaatggtgttcttcggctatgaagcaaaaggcccgggtaccccggatattgcctattatgaagcagatgccagcggtcgtattattcatgaagcctggctggaagccccgtataccagtatggttcatgattgggcagtgacccagaacttcgttgtgttcccgattattccgctgcgtagtgatattgaatggattaaacagggtcgtagttacttccagtgggacccttatgaagatgtgtatctgggcgtgatgccgcgcaaaggtaccgcaaaagatctgcgctggtatcgtggtagtaatcgcttcgcaagccatattatgggcgcatacgatgatggtcgctatatctatattgataccccggtgggccagaccaattacttcccgtggttcccggatattaccggcgccccgtatgatccggaacgtagcaaaggttatctgagccgttggaccattgatacccagggtgaaagcgatggcttcaccgaaaccagactgtgtgatctgccgggcgaattcccgcgtatggatgatcgccaggaaaccttaccgtatcattggggtattatgctgctgaccgatggcccgggtcgtgtggcaccgggtggtggcttccgctggctgatgggcattgatctgaaaaccggtcgtaaacagatctattatccgggtgataattgcaccctgggtgaagcaatcttcgttccggccgccccggatgccccgcctggtgtgggttatgtgttcaccgttgttgcacgccgcgccgaaatgcgtagtgatctggtgattctggatgcacagcatattgatggtgaaccggtgtgtaccctgagcctgccgatgcgcattcgtatgggcgtgcatggtaattgggttagtgccgaagaactggcacgtcgcaccgcataataa

SEQ ID NO: 64 - protein, 481 aa, Actinobacteria bacterium
MVRFPPPKDPFDAPMRVEADIRGVQVTQGEVPEHLDGIYYRCVSDRQWPSYVENDLELFNSDGMIISFRFHKGRVDFKSRYVRTPRFVAEEKAGRALFGAYRNPFDDDPSVAGISRGLANTNVFYHGGKLYASKEDSPPILLDPITLETIGEYDFEGALTSRTSTAHPKFDPITGEMVFFGYEAKGPGTPDIAYYEADASGRIIHEAWLEAPYTSMVHDWAVTQNFVVFPIIPLRSDIEWIKQGRSYFQWDPYEDVYLGVMPRKGTAKDLRWYRGSNRFASHIMGAYDDGRYIYIDTPVGQTNYFPWFPDITGAPYDPERSKGYLSRWTIDTQGESDGFTETRLCDLPGEFPRMDDRQETLPYHWGIMLLTDGPGRVAPGGGFRWLMGIDLKTGRKQIYYPGDNCTLGEAIFVPAAPDAPPGVGYVFTVVARRAEMRSDLVILDAQHIDGEPVCTLSLPMRIRMGVHGNWVSAEELARRTA

SEQ ID NO: 65 - DNA, 1473 bp, synthetic construct
  source 1..1473: mol_type = other DNA
atgggtattcgcttcccgaataatgacttcaaaaacttcgaagcaccgatgcgcgcagaaattgatctggccggcctggaagttgttcagggcgaagttccgaaagaactgagcggtggcttcttccgtctgattggtgatcgcaaatggccggccttcgttgaaaatgatatcttcatgctgggtgaagatggtatggccagctgcttctacttcaaagatggccgcgtggacttcaaaatgcgctatgttcgcaccccgcgcttcgttgccgaagaaaaagccggtcgcgccctgttcggcgaatatcgcaatccgttcaccgatgatccgagtgtggctggcctgagtcgtggtaccgccaatgttaccatgctggcacatgcaggcaaactgttcgcaatgaaagaagatagtccggttattgaaatggacccgttcaccctggaaaccattggcgaatatgattggggtggtgccgtgaccgcaaaagccattaccgcacatccgaaaattgatccgcgtaccggcgaactgatcttcttcggctattgcgccgccggcccggcaaccagagatgttgcatattatgaagccgatgccgaaggtcgtattattcatgaagcctggttcaaagccccgtatacctgcatggtgcatgatattatggtgaccgaacattatgttgtgttcccgattaccccgctgcgtcatgaactggaatggattgaacgccgtgaaccggcattcaaatgggaccctaatgaagaagtgtatctgggtgtgctgccgcgcaaaggcaatggtgatcagattcgttggttcaaaggtccgaatcagtgtcatggtcataccgtgggtgccttcgatgatggccgctatatctatgcagataataccttcagtaagcgtagcttcttccacttcttcccgaatgtggatggtagtgcattcagtccggccgataccatgccgtatatgaaacgtattgttattgatatggagagcgaaggtgatcacttcaccgaacagaaactgttcgaatatccgtgtgaactgccgcgtattgatacccgcttcgaaaccatggattataacttcgtgaccatgaatctgctggatgttccgggtcaggaacgcgttggccatggcttccagtgggttgcaaccatggatgtgaatggcaataaaccgagcaaaatctattatgcaggtgatgattgtagcattggtgaaccgcagttcgtgccgaaaagcaatgatgccccggaaggtgaaggctatattctgaccgttgttggtaaacatcaggaaatgcgtagtgaactgctgattctggatgccagcgatattaatgcaccgccggttgccaccgttgccctgccgttccgtctgcgtagcaccggtcatggttattggtatagcaccagtcagctgctgggtgaaaccaatgttctgggtgccagttaataa

SEQ ID NO: 66 - protein, 489 aa, Caulobacter segnis
MGIRFPNNDFKNFEAPMRAEIDLAGLEVVQGEVPKELSGGFFRLIGDRKWPAFVENDIFMLGEDGMASCFYFKDGRVDFKMRYVRTPRFVAEEKAGRALFGEYRNPFTDDPSVAGLSRGTANVTMLAHAGKLFAMKEDSPVIEMDPFTLETIGEYDWGGAVTAKAITAHPKIDPRTGELIFFGYCAAGPATRDVAYYEADAEGRIIHEAWFKAPYTCMVHDIMVTEHYVVFPITPLRHELEWIERREPAFKWDPNEEVYLGVLPRKGNGDQIRWFKGPNQCHGHTVGAFDDGRYIYADNTFSKRSFFHFFPNVDGSAFSPADTMPYMKRIVIDMESEGDHFTEQKLFEYPCELPRIDTRFETMDYNFVTMNLLDVPGQERVGHGFQWVATMDVNGNKPSKIYYAGDDCSIGEPQFVPKSNDAPEGEGYILTVVGKHQEMRSELLILDASDINAPPVATVALPFRLRSTGHGYWYSTSQLLGETNVLGAS

SEQ ID NO: 67 - DNA, 1473 bp, Streptomyces griseorubiginosus
  source 1..1473: mol_type = genomic DNA
atgcagcagatcaggtttccgcagaacgagttccagagtttcgaggcgcccatgcgcgcggaggtcgacatccgaggactggaagtagttcagggcaaggttccggagcatctcgacggcggcttctaccgactgctcggcgatcgccagtggccttccttcaccgaaaacgacatcttcctgctgaacgaagatggcatggcgtcatgcctgcgcatctcgaacggccgcgtggacttccggacacggtatgtcagaactccgcgattcgttgccgaggagcaggccgggcgggcacttttcggcgcgtaccgcaacccgttcaccgacaacccgtcagtgcaaggcgtccgccgcggaacagccaacgtgaccatgctctcgcacgccggcaagttcttcgccctcaaggaggacagcccgccggtcgagatggatcccttcaccctggagacgatcggggagtacaccttcggcggagacttcacgtccgaagcctgcaccgcccatccgaagatcgacccgcgcaccggcgaactggtgttcttcggctactgcgccaaaggcccggccacgaaagatatcgcatactacgaggcggaccgcaacgggaagatcatccacgagacctggttcgaagcaccgtacacctgcatggtgcacgacatcatggtgacccaaaactacgtggtgttccctatcacgccgctgcgtcacgaatacgaatggctggagaagcgcgagccggcgttcaagtacgacaccagcgagcaggtgcaccttggtgtactgccgcgcaagggcaccgcaaatcagattcgctggttcgtcggccccaaccagtgtcacgggcacaccgtgggcgcctatgacgacggtcgctacatctacgccgacaacacgctgagtgtccgcagcttcttcccgttcttccccaacgccgacggttcgccgttcgacccggaagacacgaaaccctacatgaagcgctgggtcatcgacatgggtagcgagggcgacactttcacggagcgtcagctgctgcagtatccctgcgaactcccgcgcatcgatacccgtttcgagacccagccctacagcttctgtgcgctgaaccttcttgatgtgcccggtcaggatcgagtcggcggcgggtggcagtggatcgcgaccatggatgtcaccggaaagaagccgacccggattcgctacgctggcgacaactgctcgatcagcgagccccagttcgttccggcgcacgacggggctgccgaaggtgaaggctatgtcctcgttgtcgtcggacgacacgacgaaatgcgcagcgagttcctgatcctcgacgcgcagaatatcgacggcccgccgattgcgaccgtcgccctgccgatgcgcgtgcgcagcacgggccacggattctggtacgggaagcgtcaactgatgggcgagacgcatcttctcggggcgaactga

SEQ ID NO: 68 - protein, 490 aa, Streptomyces griseorubiginosus
MQQIRFPQNEFQSFEAPMRAEVDIRGLEVVQGKVPEHLDGGFYRLLGDRQWPSFTENDIFLLNEDGMASCLRISNGRVDFRTRYVRTPRFVAEEQAGRALFGAYRNPFTDNPSVQGVRRGTANVTMLSHAGKFFALKEDSPPVEMDPFTLETIGEYTFGGDFTSEACTAHPKIDPRTGELVFFGYCAKGPATKDIAYYEADRNGKIIHETWFEAPYTCMVHDIMVTQNYVVFPITPLRHEYEWLEKREPAFKYDTSEQVHLGVLPRKGTANQIRWFVGPNQCHGHTVGAYDDGRYIYADNTLSVRSFFPFFPNADGSPFDPEDTKPYMKRWVIDMGSEGDTFTERQLLQYPCELPRIDTRFETQPYSFCALNLLDVPGQDRVGGGWQWIATMDVTGKKPTRIRYAGDNCSISEPQFVPAHDGAAEGEGYVLVVVGRHDEMRSEFLILDAQNIDGPPIATVALPMRVRSTGHGFWYGKRQLMGETHLLGAN

SEQ ID NO: 69 - DNA, 1548 bp, Salinibacterium sp.
  source 1..1548: mol_type = genomic DNA
atgtcccatcaaaacgacccctcggaggacgcccgagcggagcagctggcctgggtgcggaaggagccgaaggctcccatcccgacaccatcgccctaccccatgatccgctttgctgagccggcgcatccattcgacaggcccgttcgcgtcgaagctgacatcgacgggctcgagtgtgtgcagggtgccatcccggcgcaccttgacggcacgtattacaaggtcgtgagcgaccgccagttcccgtcgttcgtcgagaacgacctcggctacttcaacgacgacggcatggccctggcgtttcgattccacgacggcaaggtcacctacaagagccgattcatccggacgcctcggttccaggccgagcgcgcggccggtcgctcactgttcggcaagtatcgcaaccagttcgacgacgacgcgatggtcgaggggatgctgcgtgggaacgccaacaccaacctcttcttccacggcggcaagctgtacgcggccaaggaggactcgccgcccatcatcctggaccccgacacgctcgacacgctcggcgtgtacgactttgagggctcgctgacatccaagtcgtcgacggctcatcccaaggtcgatccactgaccggcgagatggtcttcttcggatacgccgcgaagggcgacacgacgcgggacatcgcgtactacgaggcggacagcacgggccagatcatccatgagacctggttcaaagctccctactcggcgatggtgcacgactttgccgtgacccagaactacgtcatctttccgatcatccccctgcggagcgagagggaatggatcgaggagggcaagtctgccttccaatgggatgcgagtgaggacgtctacctcggcgtgatcccacgaaagggtgacggtgcgggaatccgatggttccggggtcgctcacgcttctcgagccacatcctcggtgctttcgatgatggacggtacatctacattgacagcccggtctcggagagtaagtacttcccgtggttccccaacatggacgggacaccccacgacccggtcaaggggcagaccacgctcacgaggtggacaatcgacctccacgacggcgccgacggattcaccgagtcgcagatctcgaagctcggcgtcgagttccctcgcatggacgaccgcttcgagacgatgaagcacacctggggtgcgatggcggtcagcgctgttcccggaatcgaggtgcaggggcacggcttccgctggatctcgacaatcgactatgcgacgggcgagcagaagctgtattaccccggtgacggttcgacggtcggcgagcccgtgttcgtgccagggagcgccaacgcaccagagggacacggctacgtgttcgtgctggtatcacgccgcgatgcgatgcacagcgaactcgtcgtgctcgacgcgcagaacatcgacaaggagcccgtatgcacccttcgcatgccgctgcgcgttccgaaggggctccacggcaactgggtttcggctgaggagctcgcccagcgaacggactga

SEQ ID NO: 70 - protein, 515 aa, Salinibacterium sp.
MSHQNDPSEDARAEQLAWVRKEPKAPIPTPSPYPMIRFAEPAHPFDRPVRVEADIDGLECVQGAIPAHLDGTYYKVVSDRQFPSFVENDLGYFNDDGMALAFRFHDGKVTYKSRFIRTPRFQAERAAGRSLFGKYRNQFDDDAMVEGMLRGNANTNLFFHGGKLYAAKEDSPPIILDPDTLDTLGVYDFEGSLTSKSSTAHPKVDPLTGEMVFFGYAAKGDTTRDIAYYEADSTGQIIHETWFKAPYSAMVHDFAVTQNYVIFPIIPLRSEREWIEEGKSAFQWDASEDVYLGVIPRKGDGAGIRWFRGRSRFSSHILGAFDDGRYIYIDSPVSESKYFPWFPNMDGTPHDPVKGQTTLTRWTIDLHDGADGFTESQISKLGVEFPRMDDRFETMKHTWGAMAVSAVPGIEVQGHGFRWISTIDYATGEQKLYYPGDGSTVGEPVFVPGSANAPEGHGYVFVLVSRRDAMHSELVVLDAQNIDKEPVCTLRMPLRVPKGLHGNWVSAEELAQRTD

SEQ ID NO: 71 - DNA, 1473 bp, Streptomyces sp.
  source 1..1473: mol_type = genomic DNA
atgcagcagatcagatttccgcagaacgagttccagagcttcgaggcgcccatgcgcgcggaggtcgacatccgaggactggaagtagttcagggcgaggttccggagcatctcgacggcggcttctaccgactgctcggcgatcgacagtggccttccttcatcgaaaacgacatcttcttgctgaacgaagatggcatggcctcatgcctgcgcatttcgaacggccgcgtggacttccggacacggtatgtcaaaactccgcgattcgttgccgagcagcaggccggccgggcacttttcggcgcgtaccgcaacccgctcaccgacgacccgtcagtgcaaggcgtccgccgcggaacagccaacgtgaccatgctctcgcacgccggcaagttcttcgccctcaaggaggacagcccgccggtcgagatggatcccttcaccctggagacgatcggggagtacaccttcggcggagacttcacgtccgaagccttcaccgcccatccgaagatcgacccgcgcaccggcgaactggtgttctacggctactgcgcgaaaggcccggccacgaaagatatcgcatactacgaggcggaccggaccgggaagatcatccacgagacctggttcgaagcgccatacacctgcatggtgcacgacatcatggtgaccgaaaactacgtggtgttccctattacgccgctgcgtcacgaatacgaatggctggagaagcgcgagccggcgttcaagtacgacaccggcgagcaggtgcaccttggtgtactgccgcgcaagggcaccgcagatcagattcgctggttcgtcggccccaaccagtgtcacgggcacaccgtgggcgcctatgacgacggtcgctacatctacgccgacaacacgctgagtgtccgcagcttcttcccgttcttccccaacgccgacggttcgccgttcgacccggaagacacgaaaccctacatgaagcgctgggtcatcgacatgggtagcgagggcgacactttcatggagcgtcagctgctgcagtatccctgcgaactcccgcgcatcgatacccgtttcgagacccagccctacagcttctgtgcgctgaaccttctcgatgtgcccggtcaggatcgggtcggcggcgggtggcagtggatcgcgaccatggatgtcaccggaaagaagccgacccggattcgctatgctggcgacaactgctcgatcagcgagccccagttcgttccggcgcacgacggggctgccgaaggcgaaggctatgtcctcgttgtcgtcggacgacacgaggaaatgcgcagcgagttcctgatcctcgacgcgcagaatatcgacggcccgccgatcgcgaccgtcgccctgccgatgcgcgtgcgcagcacgggccacggattttggtacgggaagcgtcaactgatgggcgagacgcatcttctcggggcgaactga

SEQ ID NO: 72 - protein, 490 aa, Streptomyces sp.
MQQIRFPQNEFQSFEAPMRAEVDIRGLEVVQGEVPEHLDGGFYRLLGDRQWPSFIENDIFLLNEDGMASCLRISNGRVDFRTRYVKTPRFVAEQQAGRALFGAYRNPLTDDPSVQGVRRGTANVTMLSHAGKFFALKEDSPPVEMDPFTLETIGEYTFGGDFTSEAFTAHPKIDPRTGELVFYGYCAKGPATKDIAYYEADRTGKIIHETWFEAPYTCMVHDIMVTENYVVFPITPLRHEYEWLEKREPAFKYDTGEQVHLGVLPRKGTADQIRWFVGPNQCHGHTVGAYDDGRYIYADNTLSVRSFFPFFPNADGSPFDPEDTKPYMKRWVIDMGSEGDTFMERQLLQYPCELPRIDTRFETQPYSFCALNLLDVPGQDRVGGGWQWIATMDVTGKKPTRIRYAGDNCSISEPQFVPAHDGAAEGEGYVLVVVGRHEEMRSEFLILDAQNIDGPPIATVALPMRVRSTGHGFWYGKRQLMGETHLLGAN

SEQ ID NO: 73 - DNA, 1473 bp, synthetic construct
  source 1..1473: mol_type = other DNA
atgaacatccgcttcagcaataatgacttcaaatcattcgaagcaccgatgcgcgccgaaattgaactgaccggtctggaagtggtgcagggcaaagtgccgaaaaatctggaaggtggcttcttccgtctgattggtgatcgtaaatggccggcattcgtggataatgatgtgttcatgctgaatgaagatggtatggccagttgcttctacttcaaagatggcaaagtggacttccgtatgcgctatgttcgtaccccgcgcttcgttgccgaagaaaaagcaggtcgcgcactgttcggccattatcgtaatccgttcagtgatgatccgagtgtggccggcctgagtcgtggtaccgccaatgtgaccatgctgatgcatggtggtaaactgctggccctgaaagaagatagtccggccattgaactgaatccgttcaccctggaaaccattggtgaatatgattggaatggtaaagtgaccgcaaaagcaattaccgcacatccgaaaattgatgccaaaaccggtgaactggtgttcttcggctattgtgccgccggcccggcaacccgtgatattgcatattatgaagccgatagtgaaggtaatgtgacccatgaaacctggttccatgcaccgtatacctgcatggttcatgatattatggtgacccagaattatgttgtgttcccgattaccccgctgcgccatgaactggaatggattaaacgcagcgaaccggcattcaaatgggaccctcatgaaagtgtgtatctgggtgtgctgccgcgtaaaggccgcggtgatcagattcgttggttcaaaggtccgaatcagtgccatggtcataccgttggtgcatacgatgatggtcgttatatctatgcagataataccttcagtaagcgtagcttcttccacttcttcccgaatgtggatggtagccagttcaaaccgaccgatgccatgccgtatatgaaacgtgtggttattgatatggaacgcagtgataatagcttcgttgaacagccgctgttcgaatatccgtgtgaactgccgcgtattgatacccgcttcgaaaccttaccgtatagcttcgccaccatgaatctgctggatgtgccgggccaggaacgcgtgggccgtggcttccagtggattgcaaccatggatgttaatggtgcaaaaccgagcaaaattctgtatgccggcgataattgtagtattggcgaaccgcagttcgttccggcacatgataatgcagcagaaggtgatggctatattatgaccgtggtgggccgccatgaacagatgcgcagcgaactgctgattctggatgccagtgatattaatgcaccgccggtggccaccgttgccctgccgttccgtctgcgtagcaccggccatggctattggtatggtggtcgccagctgcgtggtgaaaccaacttcccgggtgcatggtaataa

SEQ ID NO: 74 - protein, 489 aa, Herbaspirillum sp.
MNIRFSNNDFKSFEAPMRAEIELTGLEVVQGKVPKNLEGGFFRLIGDRKWPAFVDNDVFMLNEDGMASCFYFKDGKVDFRMRYVRTPRFVAEEKAGRALFGHYRNPFSDDPSVAGLSRGTANVTMLMHGGKLLALKEDSPAIELNPFTLETIGEYDWNGKVTAKAITAHPKIDAKTGELVFFGYCAAGPATRDIAYYEADSEGNVTHETWFHAPYTCMVHDIMVTQNYVVFPITPLRHELEWIKRSEPAFKWDPHESVYLGVLPRKGRGDQIRWFKGPNQCHGHTVGAYDDGRYIYADNTFSKRSFFHFFPNVDGSQFKPTDAMPYMKRVVIDMERSDNSFVEQPLFEYPCELPRIDTRFETLPYSFATMNLLDVPGQERVGRGFQWIATMDVNGAKPSKILYAGDNCSIGEPQFVPAHDNAAEGDGYIMTVVGRHEQMRSELLILDASDINAPPVATVALPFRLRSTGHGYWYGGRQLRGETNFPGAW

SEQ ID NO: 75 - DNA, 1767 bp, synthetic construct
  source 1..1767: mol_type = other DNA
atggcacatatcttcgatattgccccggccgttaaaggctatgccagtggtcgcctggccaaaccgggcgatgcaaccaccttcccggataccgatgtgttccgtggtattaataaaccgagtcgcttcgaaggtgatatcttcgatctggaagttaccggcaccattccgaaagatattgatggtaccttctatcgtgttcagccggatcatcgcttcccgccgctgttcgaagatgatattcacttcaatggcgatggtagtgtgaccgcaattcgtattagcaatggccatgcagacttcaaacagcgttatgttcagaccgatcgctataaagcagaaaccgccgcccgtcgcagtctgttcggccgttatcgtaatccgtggaccgataatgaaagtgtgaaaggcgtgattcgcaccgcaagcaataccaatgttaccttctggcgcggcattctgctggcaagcaaagaagatggcccgccgttcgccatggaccctgttaccctggaaaccttaggtcgctatgacttcgaaggtcagattaaagccccgaccttcaccgcccatccgaaattcgatccggataccggcgaaatgctgtgcttcgcctatgaagcaggcggtgatggcaatgatggcagctgtgatgtggtggtgtggaccgtggatgccgatggtaaaaaaaccgaagaatgctggtataaagcaccgttcgccggtatgattcatgatatggccgtgagcaaaaacttcgtggtgctgccgctgaccccgctgaaagtgaatatggatcgtatgaaaaaaggtggtgaaaaattcgcctgggaccctaaagaagatcagtattatggcctggtgccgcgccgcaatgccaaaccggaagatattcgctggttccgcgccgataatggcatggccccgttcctgaaaaccttaagcacccattgtgccgataccccggcaagcagcgcattccagggtcatattgcaggttgttatgaaaatgaagaaggccatgttgttgtggatctgaccgttgcagatggtaatgtgttcttctggtggccgccggatgatggtaccgccattccgccgggcaaaaaaccgaatcagctgagcagcaccaccacccgctggattctggaccctaaagcagaaacaaatacccgtattaccccggcatacgtgtggccgaccaatggcgaattcagccgcattgatgatcgcttcgtgaccaaaaaatataaacacttctggcaggccaaagttgatccgacccgtccgtatgacttcgcaaaatgtggtccgccggcaggcggcctgttcaattgcctgggccattatacctggagtccggataatccgctggccaaaggtgaagaagatgtgttcttcttcggcccgaccgccaccgtgcaggaaccgagcttcattccgaaagacgaaggcggtgaaggcgatggctatctgattgcactggttaatcatctggatgttctgcgcaatgatattgcagtgttcgatgcacagcatctggccggcggcccgctggcagttctgcatctgccgctgaaactgaaactgggtctgcatggtaacttcgtggatcatcgcgatattgaagcatggcagcgccgtcgcgatgccaaaggtgatgtgggcccggttaaagttgcaaccgaaccgctgccgtggcagaaatcattctgggatcgccgccaggatggccagaatggtggcctgaatggtacccattaataa

SEQ ID NO: 76 - protein, 587 aa, Valsa mali
MAHIFDIAPAVKGYASGRLAKPGDATTFPDTDVFRGINKPSRFEGDIFDLEVTGTIPKDIDGTFYRVQPDHRFPPLFEDDIHFNGDGSVTAIRISNGHADFKQRYVQTDRYKAETAARRSLFGRYRNPWTDNESVKGVIRTASNTNVTFWRGILLASKEDGPPFAMDPVTLETLGRYDFEGQIKAPTFTAHPKFDPDTGEMLCFAYEAGGDGNDGSCDVVVWTVDADGKKTEECWYKAPFAGMIHDMAVSKNFVVLPLTPLKVNMDRMKKGGEKFAWDPKEDQYYGLVPRRNAKPEDIRWFRADNGMAPFLKTLSTHCADTPASSAFQGHIAGCYENEEGHVVVDLTVADGNVFFWWPPDDGTAIPPGKKPNQLSSTTTRWILDPKAETNTRITPAYVWPTNGEFSRIDDRFVTKKYKHFWQAKVDPTRPYDFAKCGPPAGGLFNCLGHYTWSPDNPLAKGEEDVFFFGPTATVQEPSFIPKDEGGEGDGYLIALVNHLDVLRNDIAVFDAQHLAGGPLAVLHLPLKLKLGLHGNFVDHRDIEAWQRRRDAKGDVGPVKVATEPLPWQKSFWDRRQDGQNGGLNGTH

SEQ ID NO: 77 - DNA, 1449 bp, Pseudomonas sp.
  source 1..1449: mol_type = genomic DNA
atgactactccatttcctcaaacatctgaattttccggcgcactctatacgcccagtcgcgtagaagcagaactgttcgatctcgaggttgaaggcgcccttcccgctgatgtccgaggagccttttatcaagtcgcaccagacccccagtacccaccgatgctgggcaccgatatctttttcaacggtgacggcatggtcactggcttctactttgccaatggcaaggtctcgatgcgacgacgctacgtgaaaacggaccgtctgctggcccagcgtcgtgaaggtcgttcgctcaacggcgtctatcgaaacgtctacaccaatgaccccctcgcggcgaaaaacaacaccaccgccaacaccaccgttatcccgcacaacggcgttctgttggcgctcaaggaagatgccctgccttgggcgctggatcttgaaaccctggagaccctcggcgaatggagctttgatgggcagatcaaagccgcgacgttcaccgcccaccccaagctcgatccagcgacaggcaatctcttagccttcagctatgaagccaaaggcgacggtacgcccgacattgcctatttcgaaatctcgccggacggaaaactgctacatgagatctggttccaggcgccttatgcggcgatggtgcacgatttcgcggtgaccgaacgctacgtcgtgttcccgctgattccattgacggttgacgtcgagcgcatgaaaaacggcgggccgcatttccagtggcaacccgacctgccccagctgttcgccatcgtgccgcgcaacgggtgtgggcaagatgtgcgttggttcaaggggcccaaggacggtttccaggggcatacgctcaatgcgttcgatgaggacggcaaggtgtatgtggacatgccggtcaccggcgggaacatcttctacttcttcccccaggccgacggctacgtaccaccgcctgaaaccctgaccgccagcctgatgcgctggaccttcgacctgaatggcgcgcaggaggatgttcaaccgcagccactgacagaatatccctgcgagtttccacggtgtgatgaccgctacatcggtcgaaagtaccagcatggtttcctgctcgccttcgatcctgagcgcccctacaacccagcgaatgggccaatacccttccaattcttcaacctgctggcgcacctggacctgcagacaggccgcaccgacgcctggtttcccggcgacagcggctgcttccaggagcccatcttcataccgcgctctgccgatgccgaggaagccgatggctacgtggttgccctgctcaacctcattgccgaaggacgcagcgaactggtcgtgctggacacccgtgacatggccagcgggcccattgcccggatcaggattcccttccggatgcgcatgtcgctgcatggctgctgggcaccgagcgattga

SEQ ID NO: 78 - protein, 482 aa, Pseudomonas sp.
MTTPFPQTSEFSGALYTPSRVEAELFDLEVEGALPADVRGAFYQVAPDPQYPPMLGTDIFFNGDGMVTGFYFANGKVSMRRRYVKTDRLLAQRREGRSLNGVYRNVYTNDPLAAKNNTTANTTVIPHNGVLLALKEDALPWALDLETLETLGEWSFDGQIKAATFTAHPKLDPATGNLLAFSYEAKGDGTPDIAYFEISPDGKLLHEIWFQAPYAAMVHDFAVTERYVVFPLIPLTVDVERMKNGGPHFQWQPDLPQLFAIVPRNGCGQDVRWFKGPKDGFQGHTLNAFDEDGKVYVDMPVTGGNIFYFFPQADGYVPPPETLTASLMRWTFDLNGAQEDVQPQPLTEYPCEFPRCDDRYIGRKYQHGFLLAFDPERPYNPANGPIPFQFFNLLAHLDLQTGRTDAWFPGDSGCFQEPIFIPRSADAEEADGYVVALLNLIAEGRSELVVLDTRDMASGPIARIRIPFRMRMSLHGCWAPSD

SEQ ID NO: 79 - DNA, 1449 bp, Pseudomonas brassicacearum
  source 1..1449: mol_type = genomic DNA
atgactattccatttcctcaaacatctgaattttccggcgcactctatacgcccagtcgcgtagaagcagaactgttcgaccttgaggttgaaggcacccttcccgctgagatccgaggagccttttatcaagtcgcaccagacccacagtacccaccgatgctgggcaccgatatctttttcaacggtgacggcatggtcactggtttctactttgccaatggcaaagtctcgatgcgacgccgctacgtgaaaacggaccgtctgatggcccagcgtcgtgaagggcgttcgctcaacggcgtctatcgaaacctctacaccaatgaccccctcgcggcgaaaaacaacaccaccgccaacaccaccgttatccctcacaacggcgtcctgctggcgctcaaggaagatgccctgccttgggcgatggatctcgaaaccctggagaccctcggcgaatggacctttgatgggcagatcaaagccgcgacgttcaccgcccaccccaagcgcgatccagcgacaggcaatctcttggccttcagctatgaagccaaaggcgacggtacgcccgacattgtctacttcgaaatctcgccagacggaaaactgctgcacgagatctggttccaggcgccttatgcggccatggtgcatgattttgcggtgaccgaacgctacgtcgtgttcccgttgattccgttgacggtggatgtcgaacgcatgaaaaacggcgggccgcatttccaatggcaaccggacctgcctcagctgttcgccgtcgtgccgcgcaacggtaacgcgcaagatgtgcggtggttcaagggccccaaggacagttttcaggggcatacgctcaatgcgttcgatgaggacggcaaggtgtatgtcgacatgccggtcaccggcgggaatgttttttacttctttccccaggcggacggatacgttccaccgcctgaaaccctggctgccagcctgatgcgctggacctttgacctgagcagcccccaggacgaggtcgaaccgcaaccactgaccgagtacccctgcgagtttccacggtgtgatgatcgctacatcggtcgaaagtaccagcatggtttcctgctcgcctttgatcctgagcgcccttacaacccggcgaatgggcccataccctttcaattcttcaacctgctggtccatttgaacctgaagacgggcagcaccgacgcctggtttcccggcgacagcggatgcttccaggaaccgatcttcattccgcgctcggcggatgcagaggaagccgatggctacgtggttgccctgctcaacctcatcgcagaggggcacagtgagctggtcgtgctggattcccgtgacatcgccagcggccccatcgcccgcatcaggattcccttccggatgcgcatgtcgctgcatggctgctgggcgccgagcgattga

SEQ ID NO: 80 - protein, 482 aa, Pseudomonas brassicacearum
MTIPFPQTSEFSGALYTPSRVEAELFDLEVEGTLPAEIRGAFYQVAPDPQYPPMLGTDIFFNGDGMVTGFYFANGKVSMRRRYVKTDRLMAQRREGRSLNGVYRNLYTNDPLAAKNNTTANTTVIPHNGVLLALKEDALPWAMDLETLETLGEWTFDGQIKAATFTAHPKRDPATGNLLAFSYEAKGDGTPDIVYFEISPDGKLLHEIWFQAPYAAMVHDFAVTERYVVFPLIPLTVDVERMKNGGPHFQWQPDLPQLFAVVPRNGNAQDVRWFKGPKDSFQGHTLNAFDEDGKVYVDMPVTGGNVFYFFPQADGYVPPPETLAASLMRWTFDLSSPQDEVEPQPLTEYPCEFPRCDDRYIGRKYQHGFLLAFDPERPYNPANGPIPFQFFNLLVHLNLKTGSTDAWFPGDSGCFQEPIFIPRSADAEEADGYVVALLNLIAEGHSELVVLDSRDIASGPIARIRIPFRMRMSLHGCWAPSD

SEQ ID NO: 81 - DNA, 1761 bp, synthetic construct
  source 1..1761: mol_type = other DNA
atggcccatatcttcgatctggccccggaagttagtctgccggatgaaccgatctataaagatggcaaactgcatcgtccgaatcatgtgcagttcccgcagaccccggtgttcgcaagtatgaataaaccgagccgcttcgaaggtaccattctgagcctggaacataccggtattattccgccggaaattaatggtaccttcttccgcgttcagccggatcatcgcttcccgccgatgttcgaagatgatattcacttcaatggcgatggcagcgttaccgccattcgtatcttcgatggcaaagttgacttccgccagcgttatgttcataccgaacgttataaagccgaaaccaaagcacgccgtagcctgttcggtcgctatcgtaatccgtggaccgataatgaaagcgttaaaggcgttattcgtaccgccagtaataccaatgtgttcttctggcgcggtatgctgctggccaccaaagaagatggcccgccgtatgccatggaccctgtgaccctggaaacctatggtcgttatgacttcgaaggtcagattctgagtccgaccttcaccgcccatccgaaattcgatccgcgtaccggcgaaatggtgtgcttcgcatacgaaaccggtggcgatggtgccgattgtagccgcgaagttatggtgtggaccctggataaagatggtaaaaaagttagtgaacgttggttcgaagcaccgttcgccggcatgattcatgattgcggtctgagcgaaaattggctggtgctgccgctgaccccgattaaaatggatctggaacgcatgaaacgcggtggcaataaattcgcctgggaccctaaagaagatcaggtgtatggtctggtgccgcgccgcggcgatggcgaggtgaaatggttccgtggtgaaaatgcattccatggccatgtggccggttgctatgaaaatgcacagggtcatgttgtgattgatctgaccgttgccgatggtaatgtgttcttctggttcccgccggatggtgaagaacagggtcagttcgcaaaacgtaataaactgagtagtccgacccatcgttggattctggaccctagtctgccgaataatgcccgtattaccccggcactggtgtggccgaccaatggtgaattcagtcgtattgatgatcgttggaccacccgtaaatataaacacttctggctggccaaagtggaccctagccgtccgtatgacttcgcaaaatgcggcccgccggcaggtggtctgttcaattgtctgggccattatacctgggatctggataatgaactggccaccggtcaggaagatgtgtacttcgcaggcccgacctgtaccttccaggaaccgaccttcattccgaaaggtgataaagaaggtgaaggctggctgattgcactggttaatcatctggatgtgctgcgtaatgatgttgtgattctggatgcacagaatctggcaaaaggcccggtgtgtaccattcatctgccgctgaaactgaaactgggcctgcatggcaattgggtggattggcgtgatattgaagattggaccaaacgccgccaggaagatggcgaagtgggtccggttcaggttgccaccgaaatgctgccgtggcagaaagccttctgggaaaaagaaaaagaaaagaatggcaatggtgtggaaggtccgaatattaatggtacaaatggcgccaatggcaccaatggcgttaatggcagcagccattaataa

SEQ ID NO: 82 - protein, 585 aa, Coniochaeta pulveracea
MAHIFDLAPEVSLPDEPIYKDGKLHRPNHVQFPQTPVFASMNKPSRFEGTILSLEHTGIIPPEINGTFFRVQPDHRFPPMFEDDIHFNGDGSVTAIRIFDGKVDFRQRYVHTERYKAETKARRSLFGRYRNPWTDNESVKGVIRTASNTNVFFWRGMLLATKEDGPPYAMDPVTLETYGRYDFEGQILSPTFTAHPKFDPRTGEMVCFAYETGGDGADCSREVMVWTLDKDGKKVSERWFEAPFAGMIHDCGLSENWLVLPLTPIKMDLERMKRGGNKFAWDPKEDQVYGLVPRRGDGEVKWFRGENAFHGHVAGCYENAQGHVVIDLTVADGNVFFWFPPDGEEQGQFAKRNKLSSPTHRWILDPSLPNNARITPALVWPTNGEFSRIDDRWTTRKYKHFWLAKVDPSRPYDFAKCGPPAGGLFNCLGHYTWDLDNELATGQEDVYFAGPTCTFQEPTFIPKGDKEGEGWLIALVNHLDVLRNDVVILDAQNLAKGPVCTIHLPLKLKLGLHGNWVDWRDIEDWTKRRQEDGEVGPVQVATEMLPWQKAFWEKEKEKNGNGVEGPNINGTNGANGTNGVNGSSH

SEQ ID NO: 83 - DNA, 1449 bp, Pseudomonas ogarae
  source 1..1449: mol_type = genomic DNA
atgagtattccattccctcaaacccctgaattttccggcgctctctacacgcccagccgcgtggaagcagaggtgttcgatcttgagatcgaaggcatcctgcccgcttcgatctgtgggactttttatcaggtggcgccagacccgcagtacccccccatgctgggtaacgacatttttttcaacggcgacggcgtggtcagccgcttcaactttgccaatggcaaagtctcgatgcgacgccgctacgtgaaaaccgatcgcctgctggcccagcgccgtgaagggcgttcgctcaacggcgtctatcgaaacgtctacaccaatgaccccctcgcggcgaaaaacaacaccaccgccaacaccaccgttatcccgcacaacggcgtcctgttggcgctcaaggaagatgccctgccttgggcgctggatcttgaaaccctggagaccctcggcgaatggacctttgatgggcagatcaaagccgcgacgttcaccgcccaccccaagctcgatccggtgacaggcaacctgctggcctgcagttatgaggccaaaggcgatggcacgcccgacctggcctatttcgaaatctcgcccgacggaaagctgctgcacgaaatctggttccaggccccttatgcggcgatggtgcatgatttcgcggtgaccgaacggtacgtggtgttcccgttgattccgttgacggtggacgtcgagcgcatgaaaaacggcgggccgcatttccagtggcaacccgacctgccccagctgttcgccatcgtgccgcgcaacgggtgtgggcaagatgtgcgttggttcaaggggcccaaggacggtttccaggggcatacgctcaatgcgttcgatgaggacggcaaggtgtatgtggacatgccggtcaccggcgggaacatcttctacttcttcccccaggccgacggctacgtaccaccgcctgaaaccctggccgccagcctgatgcgctggaccttcgacctcaatggcgcgcaggaggacgttcaaccgcagccactgacggaatatccctgcgaatttccacggtgtgatgatcgctacatcggtcgaaaataccagcatggtttcctgctcgcctttgatcctgagcgtccttacaacccggcgaatgggccgatacccttccagtttttcaacctgctggcgcacctggacctgcagacaggccgcaccgacgcctggtttcccggcgacagcggctgcttccaggagcccatcttcattccgcgctcggcggatgcagaggaagccgatggctatgtagttgccctgctcaacctcattgccgaaggacgcagtgaactggtcgtgctggacacccgtgacatggccagcggtcccgttgcccggatcaggatccccttccggatgcgcatgtcgctgcatggctgctgggcaccgaacgattga

SEQ ID NO: 84 - protein, 482 aa, Pseudomonas ogarae
MSIPFPQTPEFSGALYTPSRVEAEVFDLEIEGILPASICGTFYQVAPDPQYPPMLGNDIFFNGDGVVSRFNFANGKVSMRRRYVKTDRLLAQRREGRSLNGVYRNVYTNDPLAAKNNTTANTTVIPHNGVLLALKEDALPWALDLETLETLGEWTFDGQIKAATFTAHPKLDPVTGNLLACSYEAKGDGTPDLAYFEISPDGKLLHEIWFQAPYAAMVHDFAVTERYVVFPLIPLTVDVERMKNGGPHFQWQPDLPQLFAIVPRNGCGQDVRWFKGPKDGFQGHTLNAFDEDGKVYVDMPVTGGNIFYFFPQADGYVPPPETLAASLMRWTFDLNGAQEDVQPQPLTEYPCEFPRCDDRYIGRKYQHGFLLAFDPERPYNPANGPIPFQFFNLLAHLDLQTGRTDAWFPGDSGCFQEPIFIPRSADAEEADGYVVALLNLIAEGRSELVVLDTRDMASGPVARIRIPFRMRMSLHGCWAPND

SEQ ID NO: 85 - DNA, 1449 bp, Pseudomonas fluorescens
  source 1..1449: mol_type = genomic DNA
atgagtattctattccctcaaacccctgaattttccggtgttctctacacgcccagccgcgtggaagcagaggtgttcgatcttgagatcgaaggcaccctgcccgcttcgatctgtgggactttttatcaggtggcgccagacccgcagtacccccccatgctgggtaacgacatttttttcaacggcgacggcgtggtcagccgcttcaactttgccaatggcaaagtctcgatgcgacgccgctacgtgaagaccgatcgcctgctggcccagcgccgtgaagggcgttcgctcaacggagtctatcgaaacgtctacactaatgaccccctcgcggcgaaaaacaacaccaccgccaacaccaccgttatccctcacaacggcgtcctgttggcgctcaaggaagatgccctgccttgggcgctggatctcgaaaccctggagaccctcggcgaatggacctttgatgggcagatcaaagccgcgacgttcaccgcccaccccaagctcgatccggtgacaggcaacctgctggcctgcagttatgaagccaaaggcgacggcacgccagacctggcctacttcgaactctctccggacggaaagctgctgcacgagatctggttccaggcgccttatgcggcgatggtgcacgatttcgcggtgaccgaacggtacgtggtgttcccgttgattccattgacggtagacgtcgagcgcatgaaaaacggcggtccgcatttccagtggcaacccgatctgcctcaactgttcgccatcgtgccgcgctacggtagcgcgcaagacgtgcgttggttcaaggggcccaaggacggcttccaggggcacacgctcaatgcgttcgacgaggacggcaaggtgtatgtcgacatgccggtcaccggcggcaacatcttctacttttttccccaggccgacggctacgtcccaccgcctgaaaccctggccgccagcctgatgcgctggaccttcgacctgaatggcgcgcaggaggacgtccaaccgcagccactgacgaaatatccctgcgagtttccacggtgcgatgatcgctacatcggtcgaaagtaccagcatggtttcctgctcgcctttgatcctgagcgtccttacaacccggcgaatgggccgatacccttccagtttttcaacctgctggcgcacctggacctgcagacaggcctcaccgacgcctggtttcccggcgacagcggatgcttccaggaaccgatcttcattccgcgttcggcggatgcagaggaagccgatggctacgtggttgccctgctcaacctaattgccgaagggcgcaatgaactggtcgtgctggacacccgtgacatggccagcggtcccattgcccggatcagaatccccttccggatgcgcatgtcgctgcatggctgctgggcaccgagcgattga

SEQ ID NO: 86 - protein, 482 aa, Pseudomonas fluorescens
MSILFPQTPEFSGVLYTPSRVEAEVFDLEIEGTLPASICGTFYQVAPDPQYPPMLGNDIFFNGDGVVSRFNFANGKVSMRRRYVKTDRLLAQRREGRSLNGVYRNVYTNDPLAAKNNTTANTTVIPHNGVLLALKEDALPWALDLETLETLGEWTFDGQIKAATFTAHPKLDPVTGNLLACSYEAKGDGTPDLAYFELSPDGKLLHEIWFQAPYAAMVHDFAVTERYVVFPLIPLTVDVERMKNGGPHFQWQPDLPQLFAIVPRYGSAQDVRWFKGPKDGFQGHTLNAFDEDGKVYVDMPVTGGNIFYFFPQADGYVPPPETLAASLMRWTFDLNGAQEDVQPQPLTKYPCEFPRCDDRYIGRKYQHGFLLAFDPERPYNPANGPIPFQFFNLLAHLDLQTGLTDAWFPGDSGCFQEPIFIPRSADAEEADGYVVALLNLIAEGRNELVVLDTRDMASGPIARIRIPFRMRMSLHGCWAPSD

SEQ ID NO: 87 - DNA, 1446 bp, Pseudomonas syringae
  source 1..1446: mol_type = genomic DNA
atgagtattccattcccgcaaaccgctgagttttccggcgcgctctacacgcccagccgcgtagaagcagaggtgttcgaccttgaaattgaaggcgccctccccgactcgatccacgggactttttatcaggtcgcgccagacccgcaatatccacccatgctgggcaccgatatatttttcaacggtgacggaatggtcagcggcttctactttgcgcacggcaaggtatcggtgcgacgccgctacgtgaagaccgatcgcttgctggctcagcgccgtgaaggtcgctcgctcaacggcgtctatcggaacgtctttaccaatgatccgctcgcggcgaagaacaacaccaccgccaatacgaccgtgatcccacacaatggcgtcctgcttgcgctcaaggaagatgccatgccttgggcgatggatctcgaaacactggagaccctcggcgaatacaactttgacgggcagatcaagtcggcaaccttcaccgcccaccccaagctcgacccggtaacaggaaacctcttggcattcagctacgaggccaagggagacggcacgcctgacatggcgtatttcgaaatttcgcccgacggcaaactgctgcatgaaatctggtttcaggcaccttatgcggccatggtgcatgatttcgcggtaaccgaacggtatgtcgtgttcccgctcattccgttgacggtggacatcgagcgcatgaaaaacggggggccgcactttcagtggcaacccgatctgcatcagcttttcgccgttgtgccccgcaacggccttactgaagacgttcgctggttcaagggccccaaggacggtttccaggggcatacgctcaatgccttcgatgagggcggcaagatttacctcgacatgccggtcactggcgggaatatcttctacttcttcccccaggcggacggttatgtcccaccgcctgaaacgctggctgcaagcctgatgcgctggacctttgacctgaccagcgcacagaacgacattcaaccgcagccactgaccgactacccctgcgagtttccaaggtgtgatgaccgctacatcggtcgaaagtaccagcacggcttcctgctcgcctttgatccacagcgcccttacaacccggcgaacggaccgctgccctttcagtttttcaacctgctggtacatatcaacctgaagacaggaatcaccgacgcctggtttcccggtgatagcggatgcttccaggaacccattttcattccgcgctcgccagaggcggaggaagcagatggctatgtcgttgccctgctcaatctcatcgcacaagcccgtagtgaactcgttgttctggattctcgcgatatggcgggtgggcctgtcgcacgaatcaaagtcccgttccggatgcgcatgtcgctgcatggctgctgggtaccgagctga

SEQ ID NO: 88 - protein, 481 aa, Pseudomonas syringae
MSIPFPQTAEFSGALYTPSRVEAEVFDLEIEGALPDSIHGTFYQVAPDPQYPPMLGTDIFFNGDGMVSGFYFAHGKVSVRRRYVKTDRLLAQRREGRSLNGVYRNVFTNDPLAAKNNTTANTTVIPHNGVLLALKEDAMPWAMDLETLETLGEYNFDGQIKSATFTAHPKLDPVTGNLLAFSYEAKGDGTPDMAYFEISPDGKLLHEIWFQAPYAAMVHDFAVTERYVVFPLIPLTVDIERMKNGGPHFQWQPDLHQLFAVVPRNGLTEDVRWFKGPKDGFQGHTLNAFDEGGKIYLDMPVTGGNIFYFFPQADGYVPPPETLAASLMRWTFDLTSAQNDIQPQPLTDYPCEFPRCDDRYIGRKYQHGFLLAFDPQRPYNPANGPLPFQFFNLLVHINLKTGITDAWFPGDSGCFQEPIFIPRSPEAEEADGYVVALLNLIAQARSELVVLDSRDMAGGPVARIKVPFRMRMSLHGCWVPS

SEQ ID NO: 89 - DNA, 1449 bp, Pseudomonas sp.
  source 1..1449: mol_type = genomic DNA
atgagtattccattccctcaaacccctgaattttccggtgctctctacacgcccagccgcgtggaagcagaggtgttcgatcttgagatcgaaggcatcctgcctgtttcgatctgtgggactttttatcaggtggcgccagacccgcagtacccccccatgctgggtaacgacatttttttcaacggcgacggcgtggttagccgcttcaacttcgccaatggcaaagtctcgatgcgacgccgctacgtgaaaaccgatcgcctgctggcccagcgccgtgaagggcgttcactcaacggcgtctatcgaaacgtctacaccaatgaccccctcgcggcgaaaaacaacaccaccgccaacaccaccgttatccctcacaacggcgtcctgttggcgctcaaggaagatgccctgccttgggcgatggacctcgaaaccctggagaccctcggtgaatggagctttgacgggcagatcaaagccgcgacgttcactgcccaccccaagctcgatccggtgacaggcaacctgctggcctgcagttatgaggccaaaggcgacggcacgcccgacctggcctatttcgaaatctcgccggacggaaagctgctgcacgagatctggttccaggcgccttatgcggcgatggtgcatgatttcgcggtgaccgaacgctacgtcgtgttcccgttgattccgttgacggtggacgtcgaacgcatgaaaaacggcgggccacatttccagtggcaacccgacctgccccagctgttcgccatcgtgccgcgcaacgggcgtgggcaagatgtgcgctggttcaaggggcccaaggacggtttccagggacatacgctcaatgcgttcgatgaggacggcaaggtgtacgtggacatgccggtcaccggcgggaatatcttctacttcttcccccaggccgacggctacgtaccaccgcctgaaaccctggccgccagcctgatgcgctggaccttcgacctgaatggcgcgcaggaggacgttcgaccgcagccactgacggcatatccttgcgagtttccacggtgtgatgatcgctacatcggtcggaaataccggcatgggttcctgctcgcctttgaccctgagcggccttacagctcggcgaatggaccgatgcccttccagtttttcaacctgctggcgcatctggacctgcagacaggccgcaccgacgcctggtttcccggcgacagcggatgcttccaggaacccatcttcataccgcgctcggcggatgcagaggaagccgatggctacgtggttgccctgctcaacctcattgccgaagggcgcagtgaactggtggtgctggacacccgtgacatggccagcggccccattgcccggatcaagattcccttccggatgcgcatgtcgctgcatggctgctgggcaccgagcgattga

SEQ ID NO: 90 - protein, 482 aa, Pseudomonas sp.
MSIPFPQTPEFSGALYTPSRVEAEVFDLEIEGILPVSICGTFYQVAPDPQYPPMLGNDIFFNGDGVVSRFNFANGKVSMRRRYVKTDRLLAQRREGRSLNGVYRNVYTNDPLAAKNNTTANTTVIPHNGVLLALKEDALPWAMDLETLETLGEWSFDGQIKAATFTAHPKLDPVTGNLLACSYEAKGDGTPDLAYFEISPDGKLLHEIWFQAPYAAMVHDFAVTERYVVFPLIPLTVDVERMKNGGPHFQWQPDLPQLFAIVPRNGRGQDVRWFKGPKDGFQGHTLNAFDEDGKVYVDMPVTGGNIFYFFPQADGYVPPPETLAASLMRWTFDLNGAQEDVRPQPLTAYPCEFPRCDDRYIGRKYRHGFLLAFDPERPYSSANGPMPFQFFNLLAHLDLQTGRTDAWFPGDSGCFQEPIFIPRSADAEEADGYVVALLNLIAEGRSELVVLDTRDMASGPIARIKIPFRMRMSLHGCWAPSD

SEQ ID NO: 91 - DNA, 1449 bp, Pseudomonas fluorescens
  source 1..1449: mol_type = genomic DNA
atgagtattccattccctcaaacccctgaattttccggcgctctctacacgcccagccgcgtggaagcagaggtgttcgatcttgagatcgaaggcatcctgcccgcgtcgatctgtgggactttttttcaggtggcgccagacccgcagtacccccccatgctgggtaacgacatttttttcaacggcgacggcgtggttagccgcttcaactttgccaatggcaaagtctcgatgcgacgccgctacgtgaaaaccgatcgcctgctggcccagcgccgtgaagggcgttcgctcaacggcgtctatcgaaacgtctacaccaatgaccctctcgcggcgaaaaacaacaccaccgccaacaccaccgttatccctcacaacggcgtcctgttggcgctcaaggaagatgccctgccttgggcgatggacctcgaaaccctggagaccctcggcgaatggagctttgacgggcagatcaaagccgcgacgttcactgcccaccccaagctcgatccggtgacaggcaacctgctggcctgcagttatgaggccaaaggcgacggcacgcccgacctggcctatttcgaaatctcgccggacggaaagctgctgcacgagatctggttccaggcgccttatgcggcgatggtgcatgatttcgcggtgaccgaacgctacgtcgtgttcccgttgattccgttgacggtggacgtcgaacgcatgaaaaacggcgggccacattttcagtggcaacccgacctgccccagctgttcgccatcgtgccgcgcaacggccgtgggcaagatgtgcgttggttcaaggggcccaaggacggtttccaggggcatacgctcaatgcgttcgatgaggacggcaaggtgtatgtggacatgccggtcaccggcggaaatatcttctacttctttccccaggccgacggctacgtaccaccgcctgaaaccctggccgccagcctgatgcgctggaccttcgacctgaatggcgcgcaggaggacgttcaaccgcagccactgacggaatatccctgcgagtttccacggtgtgatgatcgctacatcggtcggaaataccggcatgggttcctgctcgccttcgatcctgagcgcccctacaacccggcgaatgggccaatacccttccagttcttcaacctgctggcgcacctggacctgcagacaggccgcaccgacgcctggtttcccggcgacagcggatgcttccaggaacccatcttcataccgcgctcggcggatgcggaggaagccgatggctacgtggttgccctgctcaacctcattgccgaagggcgcagtgaactggtagtgctggacacccgtgacatggctagcggccccattgcccggatcaggattcccttccggatgcgcatgtcgctgcatggctgctgggcaccaagcgattga

SEQ ID NO: 92 - protein, 482 aa, Pseudomonas fluorescens
MSIPFPQTPEFSGALYTPSRVEAEVFDLEIEGILPASICGTFFQVAPDPQYPPMLGNDIFFNGDGVVSRFNFANGKVSMRRRYVKTDRLLAQRREGRSLNGVYRNVYTNDPLAAKNNTTANTTVIPHNGVLLALKEDALPWAMDLETLETLGEWSFDGQIKAATFTAHPKLDPVTGNLLACSYEAKGDGTPDLAYFEISPDGKLLHEIWFQAPYAAMVHDFAVTERYVVFPLIPLTVDVERMKNGGPHFQWQPDLPQLFAIVPRNGRGQDVRWFKGPKDGFQGHTLNAFDEDGKVYVDMPVTGGNIFYFFPQADGYVPPPETLAASLMRWTFDLNGAQEDVQPQPLTEYPCEFPRCDDRYIGRKYRHGFLLAFDPERPYNPANGPIPFQFFNLLAHLDLQTGRTDAWFPGDSGCFQEPIFIPRSADAEEADGYVVALLNLIAEGRSELVVLDTRDMASGPIARIRIPFRMRMSLHGCWAPSD

SEQ ID NO: 93 - DNA, 1449 bp, Pseudomonas fluorescens
  source 1..1449: mol_type = genomic DNA
atgagtattccattccctcaaacccctgaattttccggtgctctctacacgcccagccgcgtggaagcagaggtgttcgatcttgagatcgaaggcatcctgcctgcttcgatctgtgggacgttttatcaggtggcgccagacccgcagtacccacccatgctgggtaacgacatttttttcaacggcgacggcatggttagccgcttcaactttgccaatggcaaagtctcgatgcgacgccgctacgtgaagaccgatcgcctgctggcccagcgccgtgaaggtcgttcgctcaacggcgtctatcgaaacgtctacaccaatgaccccttagcggcagaaaacaacaccaccgccaacaccaccgttatccctcacaacggcgtcctgttggcactcaaggaagatgccctgccttgggcgatggatctcgaaaccctggagaccctcggcgaatggacctttgatgggcagatcaaagccgcgacgttcaccgcccaccccaagctcgatccggtgacaggcaacctgctggcctgcagttatgaggccaaaggcgacggtacgcccgacctggcctacttcgaaatctcgccggacggaaagctgctgcacgagatctggttccaggccccttatgcggcgatggtgcatgatttcgcggtgaccgaacggtacgtggtgttcccattgattccgttgacggttgacgtcgagcgcatgaaaaacggcgggccgcatttccagtggcaacccgacctgcctcagctgttcgccatcgtgccgcgcaacgggcgcgggcaagatgtgcgttggttcaaggggcccaaggacggtttccaggggcatacgctcaatgcgttcgatgagggcggcaaggtgtacgtggacatgccggtcaccggcgggaatatcttctacttcttcccccaggccgacggctacgtaccaccgcctgaaaccctggccgccagcctgatgcgctggaccttcgacctgaatggcacgcaggaggacgttcaaccgcagccactgacggaatatccctgcgagtttccacggtgtgatgatcgctacatcggtcgaaaataccggcatgggttcctgctcgcctttgatcctgagcggccttacaacccggcgaatgggccgatgcccttccagtttttcaacctgctggcgcacctggacctgcagacaggccgcaccgacgcctggtttcccggcgacagcggatgcttccaggaacccatcttcataccgcgctcggcggatgcagaggaagccgatggctacgtggttgccatgctcaacctcattgccgaagggcgcagtgaactggtggtgctggacacccgtgacatggccagcggccccattgcccggatcaggattcccttccggatgcgcatgtcgctgcatggctgctgggcaccaagcgattga

SEQ ID NO: 94 - protein, 482 aa, Pseudomonas fluorescens
MSIPFPQTPEFSGALYTPSRVEAEVFDLEIEGILPASICGTFYQVAPDPQYPPMLGNDIFFNGDGMVSRFNFANGKVSMRRRYVKTDRLLAQRREGRSLNGVYRNVYTNDPLAAENNTTANTTVIPHNGVLLALKEDALPWAMDLETLETLGEWTFDGQIKAATFTAHPKLDPVTGNLLACSYEAKGDGTPDLAYFEISPDGKLLHEIWFQAPYAAMVHDFAVTERYVVFPLIPLTVDVERMKNGGPHFQWQPDLPQLFAIVPRNGRGQDVRWFKGPKDGFQGHTLNAFDEGGKVYVDMPVTGGNIFYFFPQADGYVPPPETLAASLMRWTFDLNGTQEDVQPQPLTEYPCEFPRCDDRYIGRKYRHGFLLAFDPERPYNPANGPMPFQFFNLLAHLDLQTGRTDAWFPGDSGCFQEPIFIPRSADAEEADGYVVAMLNLIAEGRSELVVLDTRDMASGPIARIRIPFRMRMSLHGCWAPSD

SEQ ID NO: 95 - DNA, 1449 bp, Pseudomonas fluorescens
  source 1..1449: mol_type = genomic DNA
atgagtattccattccctcaaacccctgaattttccggcgctctctatacgcccagccgcgtggaagcagaggtgttcgatcttgagatcgaaggcatcctgcccgcttcgatctgtgggactttttatcaggtggcgccagacccgcagtaccctcccatgctgggtaacgacattttcttcaacggcgacggcatggttagccgcttcaactttgccaatggcaaagtctcgatgcgacgccgctacgtgaagaccgatcgcctgctggcccagcgccgtgaagggcgttcgctcaacggcgtctatcgaaacgtctacaccaatgaccccctcgcggcgaaaaacaacaccaccgccaacaccaccgtcatcccccacaacggcgtcctgttggcgctcaaggaagatgccctgccttgggcgatggatctcgaaaccctggagaccctcggcgaatggagctttgatgggcagatcaaagctgcgacgttcaccgcccaccccaaactcgatccggtgacaggcaacctgctggcctgcagttatgaggccaaaggcgacggcacgcccgacctggcctatttcgaaatctcgccggacggaaagctgctgcacgagatctggttccaggcgccttatgcggccatggtgcacgatttcgcggtgaccgaacgctacgtcgtgttcccgttgattccgttgacggttgacgtcgatcgcatgaaaaacggcgggccgcatttccagtggcaacccgacctgccccagctgttcgccatcgtgccgcgcaacgggcgtgggcaagatgtgcgttggttcaaggggcccaaggacggtttccaggggcatacgctcaatgcgttcgatgaggacgacaaggtgtatgtggacatgccggtcaccggcgggaatatcttctacttcttcccccaggccgacggctacgtaccaccgcctgaaaccctggccgccagcctgatgcgctggaccttcgacctgcatggcgcgcaggaggacgttcaaccgcagccactgacggaatatccctgcgagttcccacggtgtgatgatcgctacatcggtcgaaagtaccagcatggtttcctgctcgccttcgatcctgagcgcccctacaacccggcgaatgggccaatccccttccagtttttcaacctgctggcgcacctggacctgcagacaggccgcaccgacgcctggtttcccggcgacagcggatgcttccaggaacccatcttcataccgcgctcggcggatgcagaggaagccgatggctacgtcgtagccctgctcaacctcatcgcagagggacgcagtgagctggttgtgctggacacccgtgacatggccagcgggcccattgcccgcatcaggattcccttccggatgcgcatgtcgctgcatggctgctgggcaccaatcgattga

SEQ ID NO: 96 - protein, 482 aa, Pseudomonas fluorescens
MSIPFPQTPEFSGALYTPSRVEAEVFDLEIEGILPASICGTFYQVAPDPQYPPMLGNDIFFNGDGMVSRFNFANGKVSMRRRYVKTDRLLAQRREGRSLNGVYRNVYTNDPLAAKNNTTANTTVIPHNGVLLALKEDALPWAMDLETLETLGEWSFDGQIKAATFTAHPKLDPVTGNLLACSYEAKGDGTPDLAYFEISPDGKLLHEIWFQAPYAAMVHDFAVTERYVVFPLIPLTVDVDRMKNGGPHFQWQPDLPQLFAIVPRNGRGQDVRWFKGPKDGFQGHTLNAFDEDDKVYVDMPVTGGNIFYFFPQADGYVPPPETLAASLMRWTFDLHGAQEDVQPQPLTEYPCEFPRCDDRYIGRKYQHGFLLAFDPERPYNPANGPIPFQFFNLLAHLDLQTGRTDAWFPGDSGCFQEPIFIPRSADAEEADGYVVALLNLIAEGRSELVVLDTRDMASGPIARIRIPFRMRMSLHGCWAPID

SEQ ID NO: 97 - DNA, 1443 bp, Pseudomonas brassicacearum
  source 1..1443: mol_type = genomic DNA
atgagtattccttttccacaaacccctgagttttccggcgcgctctacaaacccagccgcatagaagcagaggtgttcgacctcgagattgaaggcgttcttcccgcctcgatccatggcactttttatcaggtcgcaccggacccgcagtacccgcccatgctgggcaccgatatcttcttcaacggtgacggcatggtcagcggcttccactttgccaacggcaaggtctcgctgcgacgccgctacgtgcagacggatcgcttgctggcccaacgccgggagggtcgctcgctcaatggcgtctatcgaaatgccttcaccaacgactcgctcgcggcgaagaacaacaccaccgccaacacctccgtcatccctcacaacggcgtcctgctggcactcaaggaagatgccctgccctgggcgatggacctcgaaaccctggagaccctcggcgaatggacatttgacgggcagatcaaatcagcgacgttcaccgctcaccccaagcttgacccggcaacgggcaacctcttggccttcagctatgaggccaagggcgacggcacgcctgacctggtgtatttcgagctctcgccggacggaaagctgctgcacgagatctggttccaggccccttatgcggccatggtgcatgatttcgcggcgaccgaacggtacgtcgtattcccgctgattccgctgacggttgacgtcgagcgcatgaaaaacggcgggccgcatttccaatggcaacccgatctgcctcagcttttcgccgttgtgccccgtaacgggcgggcgcaagacgtgcgttggttcaaagggcccatggacggtttccaggggcacacgctcaatgcgttcgatgaggacggcaaggtttacgtcgacatgccggttaccggtgggaacatcttctatttcttcccccaggcggatggtcatgtaccaccgccggaaaccctggcggcctgcctgatgcgctggaccttcgacctgaacagtggcagggacgaggtcgaaccgcagccactgacggactacccctgcgagtttccaaggtgcgatgaccgctacatcggtcgacagtacgcgcacggtttcctgctcgcctttgacccggagcgtccctataacccggcgaatgggccgataccctttcagtttttcaacctgctggtccacttgaacctgaagacaggcctctcggacgcctggtttccgggcgacagcggctgcttccaggaacctatcttcattccgcgctctgctgacgccgaggaagccgatggctatgtcgttgcactgctcaacctcatcgcagaggagcgcagtgaactcgtagtgctggactctcgggacatggcgagtggtcccattgcccgaatcaggattcccttccggatgcgcatgtcgctgcatggatgctgggcgccgggt

SEQ ID NO: 98 - protein, 481 aa, Pseudomonas brassicacearum
MSIPFPQTPEFSGALYKPSRIEAEVFDLEIEGVLPASIHGTFYQVAPDPQYPPMLGTDIFFNGDGMVSGFHFANGKVSLRRRYVQTDRLLAQRREGRSLNGVYRNAFTNDSLAAKNNTTANTSVIPHNGVLLALKEDALPWAMDLETLETLGEWTFDGQIKSATFTAHPKLDPATGNLLAFSYEAKGDGTPDLVYFELSPDGKLLHEIWFQAPYAAMVHDFAATERYVVFPLIPLTVDVERMKNGGPHFQWQPDLPQLFAVVPRNGRAQDVRWFKGPMDGFQGHTLNAFDEDGKVYVDMPVTGGNIFYFFPQADGHVPPPETLAACLMRWTFDLNSGRDEVEPQPLTDYPCEFPRCDDRYIGRQYAHGFLLAFDPERPYNPANGPIPFQFFNLLVHLNLKTGLSDAWFPGDSGCFQEPIFIPRSADAEEADGYVVALLNLIAEERSELVVLDSRDMASGPIARIRIPFRMRMSLHGCWAPG

SEQ ID NO: 99 - DNA, 1689 bp, Diaporthe helianthi
  source 1..1689: mol_type = genomic DNA
atggcccacatattttctcttgcacccgccgtcaaggggtacaaggacggccggctcacaagaccaggcgaggcgacaaccttcccgcccaccccggtcttcagcggaatcaacaagccctccaggttcgaaggggacgtctttgacctcgaagtcacagggacgatccccaaggagataaacgcgaccttctaccgcatccagccggaccagcggttcccgcccttgttcgaggacgacgtccacttcaacggcgacggctccgtgacggcgatccgaatctccgatgggcacgctgactttaagcagcggtatgtgcagacggaccgttacaaggccgagacggcggcacggcggagtctgtttgggcggtacaggaacccctttacggataatgaggctgtcaagggggtgattaggacagcctcgaatactaatatcactttctggcgggggatgctgcttgccagcaaggaggacggcccgcccttcgccatggaccctgttactcttgagactctcggtcgatacgactttgagggccaaatcacgtcgccaacctttaccgcccaccccaagtttgaccccgatacgggcgagatgatctgtttcgcctatgaggccggtggaaacggcaatgacgggagccttgatattatcatgtggacgattgatgcggacggtaagaagacggaggaggcgttttacaaggcgccgtttgctggcatgatccatgatatcggcgtgagcaagaattacgtggtcctgccgctcacgccgataaaggtcaatgtggacaggatgaaacggggcggtgagaagttcgcgtgggatccttacgaggaccagtggtatggtcttgtgccgaggagaaatgggaagagcgaggacatcatttggttcagggcggataatgcttttcagggccacatagcaggttgctacgagaacgaagacggccatgtcgttgtcgacctcaccgtcgccgacgggaacgtgttcttttggtggcctccggatggttcgtacgattttcctccatttacgacctcggaactcacgcccatccagccagacaagataacagcgcgagacaagctcaacagccagacaacacgctggatcctcgacccgaaagccaagaccgagacacgaatcaagcccgcctacgtgtgggacatcaacggcgagttcagccgcatcgacgaccggtgggtgaccaagaaatacaagcacttctggcaggcgcggatcgacccttcgaaaccgtacgacttccaaaagtgcgggcccccagcgggaggactgttcaacagcctggggcactacacgtggaacccggacgacgccctgtgtcacggggaggaggacacgtacttcttcggccccacgtcgacggtgcaggagccgagcttcatcccgcgcggggaggacgcggctgagggagaggggtatttgattgcgctggtgaatcggcttgatgagctgcggaacgacgtcgccgtgtttgatgcgcagaatgttgcccaggggcccctggccgtgattcacctgcctctcaagcttaagctggggctacatggcaactttgtcgaccaccgagatattgaggcatggcagaggagacgagatgcgggtggtgatattgggcctgtcaagattgcaactgagccgttgccttggcagtag

SEQ ID NO: 100 - protein, 562 aa, Diaporthe helianthi
MAHIFSLAPAVKGYKDGRLTRPGEATTFPPTPVFSGINKPSRFEGDVFDLEVTGTIPKEINATFYRIQPDQRFPPLFEDDVHFNGDGSVTAIRISDGHADFKQRYVQTDRYKAETAARRSLFGRYRNPFTDNEAVKGVIRTASNTNITFWRGMLLASKEDGPPFAMDPVTLETLGRYDFEGQITSPTFTAHPKFDPDTGEMICFAYEAGGNGNDGSLDIIMWTIDADGKKTEEAFYKAPFAGMIHDIGVSKNYVVLPLTPIKVNVDRMKRGGEKFAWDPYEDQWYGLVPRRNGKSEDIIWFRADNAFQGHIAGCYENEDGHVVVDLTVADGNVFFWWPPDGSYDFPPFTTSELTPIQPDKITARDKLNSQTTRWILDPKAKTETRIKPAYVWDINGEFSRIDDRWVTKKYKHFWQARIDPSKPYDFQKCGPPAGGLFNSLGHYTWNPDDALCHGEEDTYFFGPTSTVQEPSFIPRGEDAAEGEGYLIALVNRLDELRNDVAVFDAQNVAQGPLAVIHLPLKLKLGLHGNFVDHRDIEAWQRRRDAGGDIGPVKIATEPLPWQ

SEQ ID NO: 101 - DNA, 1452 bp, Pseudomonas sp.
  source 1..1452: mol_type = genomic DNA
atgatcagccaatttccccaaacccctgaattttccggcgctctttatacgcccagccgcgtggaagcagaggtgttcgacctcgagattgaaggcgcccttcccgcctcgatccgtgggactttttatcaggtcgcgccagacccgcagtacccgcccttgctgggcaacgatatctttttcaacggtgacgggatggtcagcggcttcaacttcgccgacggcagggtctcgatgcgacgccgctacgtgaagactgatcgtttgatggctcagcgccgtgaaggtcgttcgctcaatggtatctatcgaaacgtctacaccaacgacccgctcgcggcgaagaacaacacgaccgcgaacaccaccgtggtgccgcacaatggcgtcctgcttgcgctcaaagaagatgccatgccctgggcaatggatctcgaaacgctggagacccttggcgaatggaattttgccgaccaggtcaaatcagcgacattcacagcccatcccaagctcgacccggcaacaggaaacctcttggcattcagctatgaagccaaaggggacggcacgcctgacctggcctatttcgaaatctcgccagacgggaaactactgcatgagatctggttccaggcaccttatgcggccatggttcatgattttgcggtgaccgaacgctacgtagtgttcccgctgattccgttgacggtggacgtcgaacgcatgaaaaacggggggccgcattttcaatggcaacccgatctaccccagcttttcgccgtggtgccacgcaatggacgcgcggaagatgttcgttggttcaacggcccgaaggacggcttccaaggccatacgctcaatgccttcgatgacaatggcaaggtttatgtagacatgccggttaccggcgggaacatcttctacttctttccacaggcggacggtcatgtgccgccacccgaaacgctggcggcctgcctgatgcgctggaccttcgacctgaacggtacgcaggacgacattcaaccgcagccattgactgactacccctgcgagtttccaaggtgtgatgaccgctacatcggtagaaagtacgaacacggtttcatactcgcctttgatccagagcgcccatacaaccctgcgaatggaccgatgccgtttcaattcttcaacctgctggctcatgtgaacctgaaaacaggaaccaccgacgcctggttccccggtgacagcggatgcttccaggagcccattttcattccgcgcgcccctgatgcacaggaagcagatggctatgtcgttgcccttctcaatctcatcgcagaaggtcgtagtgagctcgtggttctggattctcgtgacatggagagcggtcccattgcacgaatcaaaatcccattccggatgcgcatgtcgttgcacggttgctgggcaccgagcaaaaaatga

SEQ ID NO: 102 - protein, 483 aa, Pseudomonas sp.
MISQFPQTPEFSGALYTPSRVEAEVFDLEIEGALPASIRGTFYQVAPDPQYPPLLGNDIFFNGDGMVSGFNFADGRVSMRRRYVKTDRLMAQRREGRSLNGIYRNVYTNDPLAAKNNTTANTTVVPHNGVLLALKEDAMPWAMDLETLETLGEWNFADQVKSATFTAHPKLDPATGNLLAFSYEAKGDGTPDLAYFEISPDGKLLHEIWFQAPYAAMVHDFAVTERYVVFPLIPLTVDVERMKNGGPHFQWQPDLPQLFAVVPRNGRAEDVRWFNGPKDGFQGHTLNAFDDNGKVYVDMPVTGGNIFYFFPQADGHVPPPETLAACLMRWTFDLNGTQDDIQPQPLTDYPCEFPRCDDRYIGRKYEHGFILAFDPERPYNPANGPMPFQFFNLLAHVNLKTGTTDAWFPGDSGCFQEPIFIPRAPDAQEADGYVVALLNLIAEGRSELVVLDSRDMESGPIARIKIPFRMRMSLHGCWAPSKK

SEQ ID NO: 103 - DNA, 1449 bp, Pseudomonas sp.
  source 1..1449: mol_type = genomic DNA
atgagtattccattccctcaaacccctgagttttccggcgctctctacacgcccagccgggtggaagcagacgtgttcgacctggaaattgaaggcagccttcccgcttcgatccacgggacgttttatcaagtcgcaccagacccgcagtatcccccgatgctgggcaccgatatctttttcaacggtgacggcatggtcagccgcttccactttgccaacggcaaggcatcgctgcgccgtcgctacgtgaagaccgatcgcttgctggcccagcgccgtgaaggtcgctcgctcaatggcgtctatcgcaacgccttcaccaacgacccgctcgcggcgaagaacaacaccacggccaacaccaccgtcatcccacacaacggcgtgctgctggcactcaaggaagatgccctgccctgggcgatggatcgcgagaccttggaaaccctgggcgaatggagttttgacgggcagatcaaatccgcgacattcaccgcccaccccaagctcgacccgatgacgggcaatctcctggccttcagttatgaagccaaaggcgacggtacgcctgacctggcctacttcgaaatttcaccggacggaaaactgctgcacgagatctggttccaggccccttatgcagcaatggtgcacgatttcgcggtgaccgaacggtacgttgtgttcccgttgattccgttgacagtggacgtcgagcgcatgaaaaacggcggagcgcatttccagtggcaacccgacctgcctcagttgttcgccgtcgtgccgcgcaatgggcatgcgcaagatgtgcgttggttcaaggggcccaaggacggtttccaggggcatacgctcaatgcattcgatgagggcgacaaggtctatgtcgatatgccggtcaccggcgggaacatcttctatttctttccccaggccaatggttatgtcccgccgccggaaaccctggctgccagcctgatgcgctggacctttgacctgaacggcgccggggaggacgttgaaccgcaaccgctgacggattacccctgcgagtttccaaggtgtgatgaccgctacatcagtcgcaagtaccagcatggtttcctgctcgccttcgatccgcagcgcccctacaacccggcgaacggcccgataccctttcaattcttcaacctgctggtccatttgaacctgaagacaggcagcaccgacgcctggtttcctggcgacagcggctgcttccaggagcccatcttcattccccgctcggcggatgcagaggaagccgatggctacgtcgttgccctgctcaacctcatcgcagaggggcgcagtgagctggtggtgctggactcccgtgacatggccagcggtcccattgcccggatcaggattccgttccggatgcgcatgtcgctgcatggctgctgggcaccaatcgattaa

SEQ ID NO: 104 - protein, 482 aa, Pseudomonas sp.
MSIPFPQTPEFSGALYTPSRVEADVFDLEIEGSLPASIHGTFYQVAPDPQYPPMLGTDIFFNGDGMVSRFHFANGKASLRRRYVKTDRLLAQRREGRSLNGVYRNAFTNDPLAAKNNTTANTTVIPHNGVLLALKEDALPWAMDRETLETLGEWSFDGQIKSATFTAHPKLDPMTGNLLAFSYEAKGDGTPDLAYFEISPDGKLLHEIWFQAPYAAMVHDFAVTERYVVFPLIPLTVDVERMKNGGAHFQWQPDLPQLFAVVPRNGHAQDVRWFKGPKDGFQGHTLNAFDEGDKVYVDMPVTGGNIFYFFPQANGYVPPPETLAASLMRWTFDLNGAGEDVEPQPLTDYPCEFPRCDDRYISRKYQHGFLLAFDPQRPYNPANGPIPFQFFNLLVHLNLKTGSTDAWFPGDSGCFQEPIFIPRSADAEEADGYVVALLNLIAEGRSELVVLDSRDMASGPIARIRIPFRMRMSLHGCWAPID

SEQ ID NO: 105 - DNA, 1449 bp, Pseudomonas marginalis
  source 1..1449: mol_type = genomic DNA
atgaccattcaatacccccagacacctgagtttagcggcgcactctacacacccagccgggtggaagccgacgtgtttgacctggagatcgaaggcattcttcccgcttcgatccacgggactttttatcaagtcgcgccagacccgcagtatccccccatgctgggtaccgatatcttttttaacggtgacggcatggttagccgctttcattttgccaacggtaaggcctcgctgcgacgccgctacgtgaaaaccgatcgcctgctggcccagcgccgggaaggtcgctcactcaatggtgtttatcgcaacgctttcaccaatgaccccctcgcggcgaaaaacaacaccaccgccaacaccaccgtcatccctcacaacggcgtcctgttggcgctcaaggaagatgccctgccttgggcgatggatctcgaaaccctggagaccctcggcgaatggacctttgatgggcagatcaaagccgcgacgttcaccgcccaccccaagctcgatccggtgacaggcaacctgctggcctgcagttatgaagccaaaggcgacggcacgccagacctggcctacttcgaactctctccggacggaaaactgctgcacgagatctggttccaggcgccttatgcggcgatggtgcatgatttcgcggtgaccgaacggtacgtggtgttcccgttgattccgttgacagtggacgtcgagcgcatgaaaaacggcggcccgcatttccagtggcaacccgatctgcctcagctgttcgcggtcgtaccgcgtaacgggtgtgcgcaagatgtacgttggttcaaggggcccaaggacggcttccaggggcacacactgaatgcgttcgatgaggacggcaaggtgtatgtcgacatgcccgtcaccggcggcaacatcttctacttcttcccccaggccgacggctacgtaccaccgcctgaaaccctggccgccagcctgatgcgctggaccttcgacctgaatggcgcgcaggagaacgttcaaccgcagccactgacggaatatccctgcgagtttccacggtgtgatgatcgctacatcggtcgaaagtaccagcatggttttctgctcgcctttgatcctgagcgtccttacaacccggcgaatgggccgattcccttccagtttttcaacctgctggcgcacctggacctgcagacaggtcgcaccgacgcctggtttcccggcgacagcggatgcttccaggaacccatcttcataccgcgctcggcggatgcagaggaagccgatggctacgtggttgccatgctcaacctcattgccgaagggcgcagcgaactggtcgtgctggacacccgtgacatggccagcggccccattgcccgcatcaggattcccttccggatgcgcatgtcgctgcatggctgctgggcaccaagcgattga

SEQ ID NO: 106 - protein, 482 aa, Pseudomonas marginalis
MTIQYPQTPEFSGALYTPSRVEADVFDLEIEGILPASIHGTFYQVAPDPQYPPMLGTDIFFNGDGMVSRFHFANGKASLRRRYVKTDRLLAQRREGRSLNGVYRNAFTNDPLAAKNNTTANTTVIPHNGVLLALKEDALPWAMDLETLETLGEWTFDGQIKAATFTAHPKLDPVTGNLLACSYEAKGDGTPDLAYFELSPDGKLLHEIWFQAPYAAMVHDFAVTERYVVFPLIPLTVDVERMKNGGPHFQWQPDLPQLFAVVPRNGCAQDVRWFKGPKDGFQGHTLNAFDEDGKVYVDMPVTGGNIFYFFPQADGYVPPPETLAASLMRWTFDLNGAQENVQPQPLTEYPCEFPRCDDRYIGRKYQHGFLLAFDPERPYNPANGPIPFQFFNLLAHLDLQTGRTDAWFPGDSGCFQEPIFIPRSADAEEADGYVVAMLNLIAEGRSELVVLDTRDMASGPIARIRIPFRMRMSLHGCWAPSD

SEQ ID NO: 107 - DNA, 1488 bp, Pseudomonas sp.
  source 1..1488: mol_type = genomic DNA
atgaacaatcgatacccatcttcagaggttatgccgatgaccaccccgtttccccaaacccctgaattttccggcgcgctctatgcacccagtcggatagaagccgaggtgttcgacctggagattgaaggcacccttcccgcttcgatccgtggcgctttttatcaggttgcgccagacccgcaatacccgccgatgctgggctcggatattttcttcaatggcgacggaatggtcagcgcgttctactttgccgatggcaaggtgtcgatacgacgtcgctacgtaataaccgaccggttgatggctcagcgccgcgaaggtcgctcgctcaatggaatctatcgcaacgtctacaccaatgatccactggcggcgaagaacaacaccaccgccaataccagcgttgtcgctcataacggtgtgctgcttgcactcaaagaagatgccatgccctgggcaatggatcttgaaacacttgaaaccctcggcgagtggcattttgacgggcagatcgaatcagcgacgttcaccgcccaccccaagctcgacccggtaacaggaaacctcttggccttcagctacgaagccaaaggcgacggcacacctgacctggcctatttcgaactgtcgcccgacggaaagctgttgcaccaaatctggttccaggcgccgtacgcggccatggttcatgatttcgcggtgactgaacactacgtggtgttcccgctgattccgttgacgaccgacctcgagcgtatgaagaaaggtggccagcatttccaatggcaacccgatctgcctcagctcttcgccgtcgtaccacgcaatgggcgcgcggaggacgttcgatggttcaagggccccaaggacggtttccagggccatacgctcaacgcgttcgatgagggcgacaaggtttacgtagacatgccggtcaccggcgggaatatcttttacttctttccccaggccgacggtcatgtcccgccacccgaaacactggcggccagcttgatgcgctggaccttcgacctgaccagcacgcaggaggacattcaaccgcagccattgacggattacccctgcgaattcccaaggtgcgatgaccgctacatcggtcgaaaatatgagcacggctttgtgctcgcgttcgatccagagcgcccgtacaaccccgcgaatgggccgattccgtttcaattcttcaatctgctggcccatgtgaacctgaaaacaggcaccaccgatgcctggtttcccggtgacagcggatgcttccaggagcccattttcattccgcgctcccctgatgcgcaggaggcggatggctacgtcgtttccgttctcaatcacatcgcggacggtcgcagcgagcttgtagttctggattctcgtgacatggagaatggccccattgcacgaatcaaagtgccgttccggatgcgcatgtcgctgcatggttgctgggtgccgagcaaacaatga

SEQ ID NO: 108 - protein, 495 aa, Pseudomonas sp.
MNNRYPSSEVMPMTTPFPQTPEFSGALYAPSRIEAEVFDLEIEGTLPASIRGAFYQVAPDPQYPPMLGSDIFFNGDGMVSAFYFADGKVSIRRRYVITDRLMAQRREGRSLNGIYRNVYTNDPLAAKNNTTANTSVVAHNGVLLALKEDAMPWAMDLETLETLGEWHFDGQIESATFTAHPKLDPVTGNLLAFSYEAKGDGTPDLAYFELSPDGKLLHQIWFQAPYAAMVHDFAVTEHYVVFPLIPLTTDLERMKKGGQHFQWQPDLPQLFAVVPRNGRAEDVRWFKGPKDGFQGHTLNAFDEGDKVYVDMPVTGGNIFYFFPQADGHVPPPETLAASLMRWTFDLTSTQEDIQPQPLTDYPCEFPRCDDRYIGRKYEHGFVLAFDPERPYNPANGPIPFQFFNLLAHVNLKTGTTDAWFPGDSGCFQEPIFIPRSPDAQEADGYVVSVLNHIADGRSELVVLDSRDMENGPIARIKVPFRMRMSLHGCWVPSKQ

SEQ ID NO: 109 - DNA, 1452 bp, Pseudomonas sp.
  source 1..1452: mol_type = genomic DNA
atgaccaccccgtttccccaaacccctgaattttccggcgcgctctatgcacccagtcgggtagaagccgaggtgttcgacctggagattgaaggcacccttcccgcttctatccgtggcgttttttatcaggttgcgccagacccgcaatacccgccgatgctgggctcggatattttcttcaatggcgacggaatggtcagcgcgttctactttgccgatggcaaggtgtcgatgcgacgtcgctacgtaaaaaccgaccggttgatggctcagcgccgcgaaggtcgctcgctcaatggaatctatcgcaacgtctacacaaatgatccactggcggcgaagaacaacaccaccgccaataccagcgttgtcgctcataacagtgtgctgcttgcactcaaagaagatgccatgccctgggcaatggatcttgaaacactggaaaccctcggcgagtggcattttgacgggcagatcgaatcagcgacgttcaccgcccaccccaagctcgacccggtaacaggaaacctcttggccttcagctacgaagccaaaggtgacggcacacctgacctggcctatttcgaactgtcgcccgacggaaagctgttgcaccaaatctggttccaggcgccgtacgcggccatggttcatgatttcgcggtgactgaacactacgtggtgttcccgctgattccgttgacgaccgacctcgagcgtatgaagaaaggtggccagcatttccaatggcaacccgatctgcctcagctcttcgccgtcgtaccacgcaatgggcgcgcggaggacgttcgatggttcaagggccccaaggacggtttccagggccatacgctcaacgcgttcgatgagggcgacaaggtttacgtagacatgccggtcaccggcgggaatatcttttacttctttccccaggccgacggtcatgtcccgccacccgaaacactggctgccagcctgatgcgctggaccttcgacctgaccagcacgcaggaggacatgcaaccgcagccattgacggattacccctgcgaattcccaaggtgcgatgaccgctacatcggtcgaaaatatgagcacggctttgtgctcgcgttcgatcctgagcgcccgtacaacccggcgaatgggccgattccgtttcaattcttcaatctgctggcccatgtgaacctgaaaacaggcaccaccgatacctggtttcccggtgacagcggatgcttccaggagcccattttcattccgcgctcccctgatgcgcaggaggcggatggctacgtcgtttccgttctcaatcacatcgccgacggtcgcagcgagcttgtagttctggattctcgtgacatggagaatggccccattgcacgaatcaaagtgccgttccggatgcgcatgtcgctacatggttgctgggtgccgagcaaacaatga

SEQ ID NO: 110 - protein, 483 aa, Pseudomonas sp.
MTTPFPQTPEFSGALYAPSRVEAEVFDLEIEGTLPASIRGVFYQVAPDPQYPPMLGSDIFFNGDGMVSAFYFADGKVSMRRRYVKTDRLMAQRREGRSLNGIYRNVYTNDPLAAKNNTTANTSVVAHNSVLLALKEDAMPWAMDLETLETLGEWHFDGQIESATFTAHPKLDPVTGNLLAFSYEAKGDGTPDLAYFELSPDGKLLHQIWFQAPYAAMVHDFAVTEHYVVFPLIPLTTDLERMKKGGQHFQWQPDLPQLFAVVPRNGRAEDVRWFKGPKDGFQGHTLNAFDEGDKVYVDMPVTGGNIFYFFPQADGHVPPPETLAASLMRWTFDLTSTQEDMQPQPLTDYPCEFPRCDDRYIGRKYEHGFVLAFDPERPYNPANGPIPFQFFNLLAHVNLKTGTTDTWFPGDSGCFQEPIFIPRSPDAQEADGYVVSVLNHIADGRSELVVLDSRDMENGPIARIKVPFRMRMSLHGCWVPSKQ

SEQ ID NO: 111 - DNA, 1449 bp, Pseudomonas brassicacearum
  source 1..1449: mol_type = genomic DNA
atgagtattccattccctcaaacccctgaattttccggcgctctctatacgcccagccgcgtggaagcagaggtgttcgatcttgagatcgaaggcatcctgcccgcttcgatctgtgggactttttatcaggtggcgccagacccgcagtaccctcccatgctgggtaacgacattttcttcaacggcgacggcatggttagccgcttcaactttgccaatggcaaagtctcgatgcgacgccgctacgtgaagaccgatcgcctgctggcccagcgccgtgaagggcgttcgctcaacggcgtctatcgaaacgtctacaccaatgaccccctcgcggcgaaaaacaacaccaccgccaacaccaccgtcatcccccacaacggcgtcctgttggcgctcaaggaagatgccctgccttgggcgatggatctcgaaaccctggagaccctcggcgaatggagctttgatgggcagatcaaagctgcgacgttcaccgcccaccccaaactcgatccggtgacaggcaacctgctggcctgcagttatgaggccaaaggcgacggcacgcccgacctggcctatttcgaaatctcgccggacggaaagctgctgcacgagatctggttccaggcgccttatgcggccatggtgcacgatttcgcggtgaccgaacgctacgtcgtgttcccgttgattccgttgacggttgacgtcgatcgcatgaaaaacggcgggccgcatttccagtggcaacccgacctgccccagctgttcgccatcgtgccgcgcaacgggcgtgggcaagatgtgcgttggttcaaggggcccaaggacggtttccaggggcatacgctcaatgcgttcgatgaggacgacaaggtgtatgtggacatgccggtcaccggcgggaatatcttctacttcttcccccaggccgacggctacgtaccaccgcctgaaaccctggccgccagcctgatgcgctggaccttcgacctgaatggcgcgcaggaggacgttcaaccgcagccactgacggaatatccctgcgagttcccacggtgtgatgatcgctacatcggtcgaaagtaccagcatggtttcctgctcgccttcgatcctgagcgcccctacaacccggcgaatgggccaatccccttccagtttttcaacctgctggcgcacctggacctgcagacaggccgcaccgacgcctggtttcccggcgacagcggatgcttccaggaacccatcttcataccgcgctcggcggatgcagaggaagccgatggctacgtcgtagccctgctcaacctcatcgcagagggacgcagtgagctggttgtgctggacacccgtgacatggccagcgggcccattgcccgcatcaggattcccttccggatgcgcatgtcgctgcatggctgctgggcaccaatcgattga

SEQ ID NO: 112 - protein, 482 aa, Pseudomonas brassicacearum
MSIPFPQTPEFSGALYTPSRVEAEVFDLEIEGILPASICGTFYQVAPDPQYPPMLGNDIFFNGDGMVSRFNFANGKVSMRRRYVKTDRLLAQRREGRSLNGVYRNVYTNDPLAAKNNTTANTTVIPHNGVLLALKEDALPWAMDLETLETLGEWSFDGQIKAATFTAHPKLDPVTGNLLACSYEAKGDGTPDLAYFEISPDGKLLHEIWFQAPYAAMVHDFAVTERYVVFPLIPLTVDVDRMKNGGPHFQWQPDLPQLFAIVPRNGRGQDVRWFKGPKDGFQGHTLNAFDEDDKVYVDMPVTGGNIFYFFPQADGYVPPPETLAASLMRWTFDLNGAQEDVQPQPLTEYPCEFPRCDDRYIGRKYQHGFLLAFDPERPYNPANGPIPFQFFNLLAHLDLQTGRTDAWFPGDSGCFQEPIFIPRSADAEEADGYVVALLNLIAEGRSELVVLDTRDMASGPIARIRIPFRMRMSLHGCWAPID

SEQ ID NO: 113 - DNA, 1608 bp, Friedmanniomyces simplex
  source 1..1608: mol_type = genomic DNA
atggctcacatccacgacctcgctcctcagatcaaccagtactacctcggcgggcgcaagcaagacaatcagctcaagttcccggacactgatgtcttcaaaggcttcaacgcgcccagccggatcgaaggcgagatcatcaacctggaagtcgacggcaccatcccagcagatatcgatgggaccttctaccgcatccaaccggatcaccgataccctcccatgttcgaggatgacatccagttcaacggcgatggcgcggtgactgccatccggatccagaagggacatgcggatttcaagcagcgctttgtcaagaccgaccggtatgaagccgagacggctgagcgtagggcattgttcggacggtatcgtaacctctacaccgacgatgagctcgtcaaaggtgtcatcaggaccgcatcgaacaccaacgtggtcttctggcgtggcatgctgttagcgatgaaagaagacggtccaccgttcgccatggatcccgtgacgctcgagacgatcggacggtacgacttcgaaggccaggtccagtcgccgacctttacagcccacccgaagatcgatccagtgacgggcgagatggtctgctttgggtacgaggccggcgagaacggcaacgatgccagcaacgacatcgtggtgtacaccattaacgctgacggcgtgaaaacggacgagtgctggtacaagtcgccgttctgcgggatgatccacgactgtggcatctccgcgaactatctcgtgatgccactcacgccgctgaagtgctctttagacaggctgaagaaaggcggcaatcattgggcatgggatcctaatgaagatcagtggtatggtgttgttccacgaagaggcggtaggcccaacgacatcaagtggttcagatcagccaacgccttccacggccacacagcgggctgctacgagaccgaggacggtcgcatcatctacgacctcacagtcgccgacggcaacgtcttcttcttcttcccgcccgatgatatgccgccgggcagcgtgctgaagcgcaacaagctgaaaagcgagacctgccgctggatcctcgatcccaaagcgccgagcgggacttacgtgacgcctgcacaaagttggaacaccagtggcgaattctcgcggatcgacgaccgcctggtcacgaaacgatacgatcatttctggcaatgcaagatcgatccgactcgcgagtacgactttgccagctgtggaccgcccgcaggcggcctcttcaactgccttggtcactacacgtggagcggtcagacggaagatctcatcgcgctcgtgaaccagctggacgtgctgaggaacgacatcatgatcttcgatgcattgcatttgagtgctggacccgtcgcgaccatccgcttgcccttcaagctgaagctcggcttgcacggcaatttcgtcgatcatgctgatatcgcggaatgggaggcactgcggcggcgagcgggcggtatcggtccggtgaagcctgctgagaggccgctgccgtggcagctggcgatgaatgggactgcttctaacggccagtcagggcggaattgtgtcaatgggctgagccactga

SEQ ID NO: 114 - protein, 535 aa, Friedmanniomyces simplex
MAHIHDLAPQINQYYLGGRKQDNQLKFPDTDVFKGFNAPSRIEGEIINLEVDGTIPADIDGTFYRIQPDHRYPPMFEDDIQFNGDGAVTAIRIQKGHADFKQRFVKTDRYEAETAERRALFGRYRNLYTDDELVKGVIRTASNTNVVFWRGMLLAMKEDGPPFAMDPVTLETIGRYDFEGQVQSPTFTAHPKIDPVTGEMVCFGYEAGENGNDASNDIVVYTINADGVKTDECWYKSPFCGMIHDCGISANYLVMPLTPLKCSLDRLKKGGNHWAWDPNEDQWYGVVPRRGGRPNDIKWFRSANAFHGHTAGCYETEDGRIIYDLTVADGNVFFFFPPDDMPPGSVLKRNKLKSETCRWILDPKAPSGTYVTPAQSWNTSGEFSRIDDRLVTKRYDHFWQCKIDPTREYDFASCGPPAGGLFNCLGHYTWSGQTEDLIALVNQLDVLRNDIMIFDALHLSAGPVATIRLPFKLKLGLHGNFVDHADIAEWEALRRRAGGIGPVKPAERPLPWQLAMNGTASNGQSGRNCVNGLSH

SEQ ID NO: 115 - DNA, 1449 bp, Pseudomonas brassicacearum
  source 1..1449: mol_type = genomic DNA
atgagtattccttttccacaaacccctgagttttccggcgcgctctacaaacccagccgcatagaagcagaggtgttcgacctcgagattgaaggcgttcttcccgcctcgatccatggcactttttatcaggtcgcaccggacccgcagtacccgcccatgctgggcaccgatatcttcttcaacggtgacggcatggtcagcggcttccactttgccaacggcaaggtctcgctgcgacgccgctacgtgcagacggatcgcttgctggcccaacgccgggagggtcgctcgctcaatggcgtctatcgaaatgccttcaccaacgactcgctcgcggcgaagaacaacaccaccgccaacacctccgtcatccctcacaacggcgtcctgctggcactcaaggaagatgccctgccctgggcgatggacctcgaaaccctggagaccctcggcgaatggacatttgacgggcagatcaaatcagcgacgttcaccgctcaccccaagcttgacccggcaacgggcaacctcttggccttcagctatgaggccaagggcgacggcacgcctgacctggtgtatttcgagctctcgccggacggaaagctgctgcacgagatctggttccaggccccttatgcggccatggtgcatgatttcgcggcgaccgaacggtacgtcgtattcccgctgattccgctgacggttgacgtcgagcgcatgaaaaacggcgggccgcatttccaatggcaacccgatctgcctcagcttttcgccgttgtgccccgtaacgggcgggcgcaagacgtgcgttggttcaaagggcccatggacggtttccaggggcacacgctcaatgcgttcgatgaggacggcaaggtttacgtcgacatgccggttaccggtgggaacatcttctatttcttcccccaggcggatggtcatgtaccaccgccggaaaccctggcggcctgcctgatgcgctggaccttcgacctgaacagtggcagggacgaggtcgaaccgcagccactgacggactacccctgcgagtttccaaggtgcgatgaccgctacatcggtcgacagtacgcgcacggtttcctgctcgcctttgacccggagcgtccctataacccggcgaatgggccgataccctttcagtttttcaacctgctggtccacttgaacctgaagacaggcctctcggacgcctggtttccgggcgacagcggctgcttccaggaacctatcttcattccgcgctctgctgacgccgaggaagccgatggctatgtcgttgcactgctcaacctcatcgcagaggagcgcagtgaactcgtagtgctggactctcgggacatggcgagtggtcccattgcccgaatcaggattcccttccggatgcgcatgtcgctgcatggatgctgggcgccgggttcctga

SEQ ID NO: 116 - protein, 482 aa, Pseudomonas brassicacearum
MSIPFPQTPEFSGALYKPSRIEAEVFDLEIEGVLPASIHGTFYQVAPDPQYPPMLGTDIFFNGDGMVSGFHFANGKVSLRRRYVQTDRLLAQRREGRSLNGVYRNAFTNDSLAAKNNTTANTSVIPHNGVLLALKEDALPWAMDLETLETLGEWTFDGQIKSATFTAHPKLDPATGNLLAFSYEAKGDGTPDLVYFELSPDGKLLHEIWFQAPYAAMVHDFAATERYVVFPLIPLTVDVERMKNGGPHFQWQPDLPQLFAVVPRNGRAQDVRWFKGPMDGFQGHTLNAFDEDGKVYVDMPVTGGNIFYFFPQADGHVPPPETLAACLMRWTFDLNSGRDEVEPQPLTDYPCEFPRCDDRYIGRQYAHGFLLAFDPERPYNPANGPIPFQFFNLLVHLNLKTGLSDAWFPGDSGCFQEPIFIPRSADAEEADGYVVALLNLIAEERSELVVLDSRDMASGPIARIRIPFRMRMSLHGCWAPGS

SEQ ID NO: 117 - DNA, 1449 bp, Pseudomonas sp.
  source 1..1449: mol_type = genomic DNA
atgagtattccattccctcaaacccctgaattttccggtgctctctacacgcccagccgcgtggaagcagaggtgttcgatcttgagatcgaaggcatcctgcctgtttcgatctgtgggactttttatcaggtggcgccagacccgcagtacccccccatgctgggtaacgacatttttttcaacggcgacggcgtggttagccgcttcaactttgccaatggcaaagtctcgatgcgacgccgctacgtgaaaaccgatcgcctgctggcccagcgccgtgaagggcgttcgctcaacggcgtctatcgaaacgtctacaccaatgaccccctcgcggcgaaaaacaacaccaccgccaacaccaccgttatccctcacaacggcgtcctgttggcgctcaaggaagatgccctgccttgggcgatggacctcgaaaccctggagaccctcggcgaatggagctttgacgggcagatcaaagccgcgacgttcactgcccaccccaagctcgatccggtgacaggcaacctgctggcctgcagttatgaggccaaaggcgacggcacgcccgacctggcctatttcgaaatctcgccggacggaaaactgctgcacgagatctggttccaggcgccttatgcggcgatggtgcatgatttcgcggtgaccgaacgctacgtcgtgttcccgttgattccgttgacggtggacgtcgaacgcatgaaaaacggcgggccacatttccagtggcaacccgacctgccccagctgttcgccatcgtgccgcgcaacgggcgtgggcaagatgtgcgttggttcaaggggcccaaggacggtttccaggggcatacgctcaatgcgttcgatgaggacggcaaggtgtatgtggacatgccggtcaccggcgggaatatcttctacttcttccctcaggccgacggctacgtaccaccgcctgaaaccctggccgccagcctgatgcgctggaccttcgacctgaatggcgcgcaggaggacgttcaaccgcagccactgacggcatatccctgcgagtttccacggtgtaatgatcgctacatcggtcggaaataccggcatgggttcctgctcgcctttgaccctgagcggccttacagcccggcgaatggaccgatgcccttccagtttttcaacctgctggcccatctggacctgcagacaggccgcaccgacgcctggtttcccggcgacagcggatgcttccaggaacccatcttcataccgcgctcggcggatgcagaggaagccgatggctacgtggttgccctgctcaacctcattgccgaagggcgcagtgaactggtggtgctggacacccgtgacatggccagcggccccattgcccggatcaggattcccttccggatgcgcatgtcgctgcatggctgctgggcaccgagcgattaa

SEQ ID NO: 118 - protein, 482 aa, Pseudomonas sp.
MSIPFPQTPEFSGALYTPSRVEAEVFDLEIEGILPVSICGTFYQVAPDPQYPPMLGNDIFFNGDGVVSRFNFANGKVSMRRRYVKTDRLLAQRREGRSLNGVYRNVYTNDPLAAKNNTTANTTVIPHNGVLLALKEDALPWAMDLETLETLGEWSFDGQIKAATFTAHPKLDPVTGNLLACSYEAKGDGTPDLAYFEISPDGKLLHEIWFQAPYAAMVHDFAVTERYVVFPLIPLTVDVERMKNGGPHFQWQPDLPQLFAIVPRNGRGQDVRWFKGPKDGFQGHTLNAFDEDGKVYVDMPVTGGNIFYFFPQADGYVPPPETLAASLMRWTFDLNGAQEDVQPQPLTAYPCEFPRCNDRYIGRKYRHGFLLAFDPERPYSPANGPMPFQFFNLLAHLDLQTGRTDAWFPGDSGCFQEPIFIPRSADAEEADGYVVALLNLIAEGRSELVVLDTRDMASGPIARIRIPFRMRMSLHGCWAPSD

SEQ ID NO: 119 - DNA, 1455 bp, synthetic construct
  source 1..1455: mol_type = other DNA
atgacccatccgttcccgcagaccccggaattcagtggcgcactgtatgcaccgagccgcgttgaagcagaagtgttcgatctggaagtggaaggtaccctgccggatagcattcgtggtgccttctatcaggttgcaccggaccctcagtatccgccgatgctgggtaatgatatcttcttcaatggtgatggtatggttagcgccttcaacttcgccgatggcaaagttagtatgcgtcgtcgctatgttaaaaccgatcgcctgatggcacagcgccgtgaaggccgcagtctgaatggtgtgtatcgcaatgtgttcaccaatgatccgctggcagcaaaaaataataccaccgccaataccagcgtggtggcacataatggtgtgctgctggcactgaaagaagatgcactgccgtgggcaatggatctggaaaccttagaaaccttaggcgaatggagcttcgatggtcagattgaaagtgcaaccttcaccgcacatccgaaactggaccctgtgaccggtaatctgctggcattcagttatgaagcaaaaggcgatggcaccccggatctggcctacttcgaactgagtccggatggcaaactgctgcatcagatctggttccaggcaccgtatgcagcaatggtgcatgacttcgcagtgaccgaacattatgttgtgttcccgctgattccgctgaccgttgatgttgaacgtatgaaaaatggcggtcagcacttccagtggcagccggatctgccgcagctgttcgcagttgtgccgcgtaatggccatgccgaagatgttcgctggttcaaaggcccgaaagatggcttccagggtcataccctgaatgcattcgatgaagatgataaagtgtatgtggatatgccggttaccggtggtaatatcttctacttcttcccgcaggcagatggttatgttccgccgccggaaaccttagcagcaagtctgatgcgctggaccttcgatctgaccagcacccaggatgatattcagccgcagccgctgaccgattatccgtgcgaattcccgcgttgcgatgatcgctatattggccgtaaatatgaacatggcttcgttctggccttcgatccggaacgcccgtataatccggccaatggcccgattccgttccagttcttcaatctgctggcccgtgtgaatctgaaaaccggcgcaaccgatgcatggttcccgggtgatagcggttgcttccaggaaccgatcttcattccgcgtgcaccggatgcacaggaagcagatggctatgttgttgcactgctgaatctgattgccgaaggtcgtagtgaactggtggtgctggatagtcgcgatatggccagtggcccgattgcccgtattaaagttccgttccgtctgcgtatgagtctgcatggctgctgggttccgagtaaaaaataataa

SEQ ID NO: 120 - protein, 483 aa, Pseudomonas frederiksbergensis
MTHPFPQTPEFSGALYAPSRVEAEVFDLEVEGTLPDSIRGAFYQVAPDPQYPPMLGNDIFFNGDGMVSAFNFADGKVSMRRRYVKTDRLMAQRREGRSLNGVYRNVFTNDPLAAKNNTTANTSVVAHNGVLLALKEDALPWAMDLETLETLGEWSFDGQIESATFTAHPKLDPVTGNLLAFSYEAKGDGTPDLAYFELSPDGKLLHQIWFQAPYAAMVHDFAVTEHYVVFPLIPLTVDVERMKNGGQHFQWQPDLPQLFAVVPRNGHAEDVRWFKGPKDGFQGHTLNAFDEDDKVYVDMPVTGGNIFYFFPQADGYVPPPETLAASLMRWTFDLTSTQDDIQPQPLTDYPCEFPRCDDRYIGRKYEHGFVLAFDPERPYNPANGPIPFQFFNLLARVNLKTGATDAWFPGDSGCFQEPIFIPRAPDAQEADGYVVALLNLIAEGRSELVVLDSRDMASGPIARIKVPFRLRMSLHGCWVPSKK

SEQ ID NO: 121 - DNA, 1452 bp, Pseudomonas sp.
  source 1..1452: mol_type = genomic DNA
atgaccagccaatttccccaaacccctgaattttccggcgctctttatacgcccagccgcgtggaagcagaggtgttcgacctcgagattgaaggcacccttcccgcctcgatccgtgggactttttatcaggtcgcgccagacccgcagtacccgcccttgctgggcagcgatatctttttcaacggtgacgggatggtcagcggcttcaacttcgccgacggcagggtctcgatgcgacgccgctacgtgaagactgatcgtttgctggctcagcgccgtgaaggtcgttcgctcaatggcatctatcgaaacgtctacaccaatgacccgctcgcggcgaagaacaacacgaccgcgaacaccaccgtggtgccgcacaatggcgtcctgcttgcgctcaaagaagatgccatgccctgggcaatggatctcgaaacgctggagacccttggcgaatggaattttgacgaccaggtcaaatcagcgacattcacagcccatcccaagctcgacccggcaacaggaaacctcttggcattcagctatgaagccaaaggggacggcacgcctgacctggcctatttcgaaatctcgccggacgggaaactactgcatgagatctggttccaggcaccttattcggccatggttcatgattttgcggtgaccgaacgctacgtagtgttcccgctgattccgttgacggtggacgtcgaacgcatgaaaaacggggggccgcattttcaatggcaacccgatctacctcagcttttcgccgtggtgccacgggatggccacgcggaagatgttcgttggttcaagggcccgaaggacggcttccaaggccatacgctcaatgccttcgatgacaatggcaaggtttatgtagacatgccggttaccggcgggaacatcttctacttctttccacaggcggacggtcatgtgccgccacccgaaacgctggcggcctgcctgatgcgctggaccttcgacctgagcggtacgcaggatgacattcaaccgcagccattgactgactatccctgcgagtttccaaggtgtgatgaccgctacatcggtcgaaagtacgaacacggcttcatactcgcctttgatccagagcgcccatacaaccctgcgaatggaccgatgccgtttcaattcttcaacctgctggctcatgtgaacctgaaaacaggaaccaccgacgcctggttccccggtgacagcggatgcttccaggagcccatttttattccgcgctcccctgatgcggcggaagcagatggctatgtcgttgcccttctcaatctcatcgcagaaggtcgtagtgagctcgtggttctggattctcgtgacatggagagcggccccattgcacgaatcaaaatcccgttccgaatgcgcatgtcgttgcacggttgctgggcaccgagcaaaaaatga

SEQ ID NO: 122 - protein, 483 aa, Pseudomonas sp.
MTSQFPQTPEFSGALYTPSRVEAEVFDLEIEGTLPASIRGTFYQVAPDPQYPPLLGSDIFFNGDGMVSGFNFADGRVSMRRRYVKTDRLLAQRREGRSLNGIYRNVYTNDPLAAKNNTTANTTVVPHNGVLLALKEDAMPWAMDLETLETLGEWNFDDQVKSATFTAHPKLDPATGNLLAFSYEAKGDGTPDLAYFEISPDGKLLHEIWFQAPYSAMVHDFAVTERYVVFPLIPLTVDVERMKNGGPHFQWQPDLPQLFAVVPRDGHAEDVRWFKGPKDGFQGHTLNAFDDNGKVYVDMPVTGGNIFYFFPQADGHVPPPETLAACLMRWTFDLSGTQDDIQPQPLTDYPCEFPRCDDRYIGRKYEHGFILAFDPERPYNPANGPMPFQFFNLLAHVNLKTGTTDAWFPGDSGCFQEPIFIPRSPDAAEADGYVVALLNLIAEGRSELVVLDSRDMESGPIARIKIPFRMRMSLHGCWAPSKK

SEQ ID NO: 123 - DNA, 1449 bp, Pseudomonas kilonensis
  source 1..1449: mol_type = genomic DNA
atgagtattccatttcctcaaacccctgaattttccggcgctctctacacgcccagccgcgtggaagcagaggtgttcgatcttgagatcgaaggcatcctgcccgcttcgatctgtgggactttttatcaggtggcgccagacccgcagtacccccccatgctgggtaacgacatttttttcaacggcgacggcgtggtcagccgcttcaactttgccaatggcaaagtctcgatgcgacgccgctacgtgaaaaccgatcgcctgctggcccagcgccgtgaagggcgttcgctcaacggcgtctatcgaaacgtctacaccaatgaccccctcgcggcgaaaaacaacaccaccgccaacaccaccgttatcccgcacaacggcgtcctgttggcgctcaaggaagatgccctgccttgggcgctggatcttgaaaccctggagaccctcggcgaatggacctttgatgggcagatcaaagccgcgacgttcaccgcccaccccaagctcgatccggtgacaggcaacctgctggcctgcagttatgaggccaaaggcgatggcacgcccgacctggcctatttcgaaatctcgcccgacggaaagctgctgcacgaaatctggttccaggccccttatgcggcgatggtgcatgatttcgcggtgaccgaacggtacgtggtgttcccgttgattccgttgacggtggacgtcgagcgcatgaaaaacggcgggccgcatttccagtggcaacccgacctgccccagctgttcgccatcgtgccgcgcaacgggtgtgggcaagatgtgcgttggttcaaggggcccaaggacggtttccaggggcatacgctcaatgcgttcgatgaggacggcaaggtgtatgtggacatgccggtcaccggcgggaacatcttctacttcttcccccaggccgacggctacgtaccaccgcctgaaaccctggccgccagcctgatgcgctggaccttcgacctcaatggcgcgcaggaggacgttcaaccgcagccactgacggaatatccctgcgaatttccacggtgtgatgatcgctacatcggtcgaaaataccagcatggtttcctgctcgcctttgatcctgagcgtccttacaacccggcgaatgggccgatacccttccagtttttcaacctgctggcgcacctggacctgcagacaggccgcaccgacgcctggtttcccggcgacagcggctgcttccaggagcccatcttcattccgcgctcggcggatgcagaggaagccgatggctacgtggttgccctgctcaacctcattgccgaagggcgcagtgaactggtcgtgctggacacccgtgacatggccagcggtcccattgcccggatcaggatccccttccggatgcgcatgtcgctgcatggctgctgggcaccgaacgattga

SEQ ID NO: 124 - protein, 482 aa, Pseudomonas kilonensis
MSIPFPQTPEFSGALYTPSRVEAEVFDLEIEGILPASICGTFYQVAPDPQYPPMLGNDIFFNGDGVVSRFNFANGKVSMRRRYVKTDRLLAQRREGRSLNGVYRNVYTNDPLAAKNNTTANTTVIPHNGVLLALKEDALPWALDLETLETLGEWTFDGQIKAATFTAHPKLDPVTGNLLACSYEAKGDGTPDLAYFEISPDGKLLHEIWFQAPYAAMVHDFAVTERYVVFPLIPLTVDVERMKNGGPHFQWQPDLPQLFAIVPRNGCGQDVRWFKGPKDGFQGHTLNAFDEDGKVYVDMPVTGGNIFYFFPQADGYVPPPETLAASLMRWTFDLNGAQEDVQPQPLTEYPCEFPRCDDRYIGRKYQHGFLLAFDPERPYNPANGPIPFQFFNLLAHLDLQTGRTDAWFPGDSGCFQEPIFIPRSADAEEADGYVVALLNLIAEGRSELVVLDTRDMASGPIARIRIPFRMRMSLHGCWAPND

SEQ ID NO: 125 - DNA, 1446 bp, Pseudomonas syringae
  source 1..1446: mol_type = genomic DNA
atgactattccattccctcaaacccctgagttttccggtgcgctctatacgcccagccgcgtagaagcagaagtgttcgatctcgagattgaaggcgcccttcccgcctcgatccgtgggactttttatcaggtcgcgccagacccgcagtacccaccgatgctgggcaccgatatctttttcaacggtgatggaatggtcagcgggttctacttcgcaaacggcaaggtttcgctgcgacgccgctacgtgaagacggaccggttgctggcccagcgtcgtgaaggtcgttcgctcaatggtatctatcgaaacgtctacaccaatgatccactcgcggcgaagaacaacaccaccgcgaacaccaccgtcgtgccacacaacggtgtcttgctcgcactcaaggaagatgccatgccttgggcaatggatctcgaaacgctggagaccctcggcgaatggaattttgacgggcagatcaaatcagcgacgttcaccgcacaccccaagctcgacccggcaacaggaaacctcttggcattcagctatgaagccaaaggagaaggcacgcctgacctggcctatttcgaactctcgccagacggaaagctgctgcatgagatctggttccaggcaccttatgcagccatggttcatgatttcgcggtgaccgaacactacgtcgtgttcccgctcattccgttaacagtggacatcgaacgcatgaaaaacggggggccgcacttccaatggcaacccgatttacctcagcttttcgctgtcgtgccacgcaacggatgtgcgcaagacgttcgctggttcaagggccccaaggaaggtttccagggtcatacactcaatgcgttcgatgaggacggcaaggtttacgtcgatatgccggtcaccggagggaacatcttctactttttcccccaggcggacggttatgtcccaccgcctgaaacgctagccgccagcctgatgcgctggacctttgacctgaatggttcgcaggacgaaatccaaccgcagccactgactgactacccctgtgagtttccaaggtgtgatgaccgctacatcggtcgaaagtacgagcacggcttcctgctcgcctttgatccgcagcgcccctacaaccccgagaatggaccgataccgtttcagtttttcaacttgctggctcacgtgaacctgaaaacaggaaccaccgacgcctggtttcccggtgacagcggatgcttccaggagcccattttcattccgcgctccccaaatgcggaggaagccgatggctatgtcgtttccctactcaatctcatcgcagagggccgtagtgagctcgtcgttcttgactctcgtgacatggagagtggcccgattgcgcgaatcaaagtcccgttccggatgcgcatgtcgctgcacgggtgctgggcacccagctga

SEQ ID NO: 126 - protein, 481 aa, Pseudomonas syringae
MTIPFPQTPEFSGALYTPSRVEAEVFDLEIEGALPASIRGTFYQVAPDPQYPPMLGTDIFFNGDGMVSGFYFANGKVSLRRRYVKTDRLLAQRREGRSLNGIYRNVYTNDPLAAKNNTTANTTVVPHNGVLLALKEDAMPWAMDLETLETLGEWNFDGQIKSATFTAHPKLDPATGNLLAFSYEAKGEGTPDLAYFELSPDGKLLHEIWFQAPYAAMVHDFAVTEHYVVFPLIPLTVDIERMKNGGPHFQWQPDLPQLFAVVPRNGCAQDVRWFKGPKEGFQGHTLNAFDEDGKVYVDMPVTGGNIFYFFPQADGYVPPPETLAASLMRWTFDLNGSQDEIQPQPLTDYPCEFPRCDDRYIGRKYEHGFLLAFDPQRPYNPENGPIPFQFFNLLAHVNLKTGTTDAWFPGDSGCFQEPIFIPRSPNAEEADGYVVSLLNLIAEGRSELVVLDSRDMESGPIARIKVPFRMRMSLHGCWAPS

SEQ ID NO: 127 - DNA, 1452 bp, synthetic construct
  source 1..1452: mol_type = other DNA
atgaccattccgttcccgcagaccagcgaattcagtggcgcactgtataccccgagtcgtgtggaagccgaactgttcgatctggaagtggaaggcaccctgccggcagaaattcgcggtgccttctatcaggtggcaccggaccctcagtatccgccgatgctgggtaccgatatcttcttcaatggtgatggtatggtgaccggcttctacttcgcaaatggcaaagtgagcatgcgtcgtcgctatgttaaaaccgatcgtctgatggcacagcgtcgtgaaggtcgcagtctgaatggtgtgtatcgcaatctgtataccaatgatccgctggccgcaaaaaataataccaccgccaataccaccgttattccgcataatggtgttctgctggccctgaaagaagatgccctgccgtgggcaatggatctggaaaccttagaaaccttaggtgaatggaccttcgatggtcagattaaagcagcaaccttcaccgcacatccgaaacgcgatccggcaaccggcaatctgctggcattcagttatgaagcaaaaggcgatggcaccccggatattgcctacttcgaaattagtccggatggcaaactgctgcatgaaatctggttccaggcaccgtatgccgccatggttcatgacttcgccgtgaccgaacgttatgtggtgttcccgctgattccgctgaccgttgatgtggaacgtatgaaaaatggcggcccgcacttccagtggcagccggatctgccgcagctgttcgcagtggtgccgcgtaatggcaatgcacaggatgtgcgctggttcaaaggtccgaaagatagcttccagggtcataccctgaatgcattcgatgaagatggcaaagtgtatgttgatatgccggttaccggcggtaatgtgttctacttcttcccgcaggccgatggctatgtgccgccgcctgaaaccttagcagcaagtctgatgcgctggaccttcgacctgagcagtccgcaggatgaagtggaaccgcagccgctgaccgaatatccgtgcgaattcccgcgttgcgatgatcgctatattggccgcaaatatcagcatggcttcctgctggccttcgatccggaacgcccgtataatccggccaatggtccgattccgttccagttcttcaatctgctggtgcatctgaatctgaaaaccggcagcaccgatgcatggttcccgggtgatagcggttgcttccaggaaccgatcttcattccgcgcagtgcagatgcagaagaagcagatggttatgttgttgccctgctgaatctgattgccgaaggccgtagtgaactggttgtgctggatagccgcgatatggccggtggcccgattgcccgtattcgcattccgttccgtatgcgcatgagtctgcatggttgttgggcaccgagcgattaataa

SEQ ID NO: 128 - protein, 482 aa, Pseudomonas kilonensis
MTIPFPQTSEFSGALYTPSRVEAELFDLEVEGTLPAEIRGAFYQVAPDPQYPPMLGTDIFFNGDGMVTGFYFANGKVSMRRRYVKTDRLMAQRREGRSLNGVYRNLYTNDPLAAKNNTTANTTVIPHNGVLLALKEDALPWAMDLETLETLGEWTFDGQIKAATFTAHPKRDPATGNLLAFSYEAKGDGTPDIAYFEISPDGKLLHEIWFQAPYAAMVHDFAVTERYVVFPLIPLTVDVERMKNGGPHFQWQPDLPQLFAVVPRNGNAQDVRWFKGPKDSFQGHTLNAFDEDGKVYVDMPVTGGNVFYFFPQADGYVPPPETLAASLMRWTFDLSSPQDEVEPQPLTEYPCEFPRCDDRYIGRKYQHGFLLAFDPERPYNPANGPIPFQFFNLLVHLNLKTGSTDAWFPGDSGCFQEPIFIPRSADAEEADGYVVALLNLIAEGRSELVVLDSRDMAGGPIARIRIPFRMRMSLHGCWAPSD

SEQ ID NO: 129 - DNA, 1455 bp, synthetic construct
  source 1..1455: mol_type = other DNA
atgaccattccgttcccgcagaccccggaattcagcggtgccctgtataccccgagccgtgtggaagcagaagtgttcgatctggaaattgaaggcgttctgccggcaagcattggcggcaccttctatcaggttgcaccggaccctcagtatccgccgatgctgggtaatgatatcttcttcaatggtgatggtgtggttagtcgcttcaacttcgcaaatggtaaagttagtatgcgccgccgctatgtgaaaaccgatcgtctgctggcacagcgccgtgaaggtcgtagcctgaatggcgtgtatcgcaatgtgtataccaatgatcgcctggcagcaaaaaataataccaccgccaataccaccgtgattccgcataatggtgttctgctggccctgaaagaagatgccctgccgtgggcaatggatctggataccctggaaaccttaggcgaatggaccttcgatggccagattaaaagcgcaaccttcaccgcacatccgaaactggaccctgtgaccggtaatctgctggcatgcagctatgaagccaaaggcgatggtaccccggatctggcctacttcgaaattagcccggatggcaaactgctgcatgaaatctggttccaggcaccgtatgccgccatggttcatgacttcgccgttaccgaacgttatgtggtgttcccgctgattccgctgaccgtggatgtggaacgtatgaaaaatggcggcccgcacttccagtggcagccggatctgccgcagctgttcgccgtggtgccgcgtaatggtaatgcccaggatgttcgttggttcaaaggtccgaaagatggcttccagggtcataccctgaatgccttcgatgaagatggcaaagtgtatgtggatatgccggttaccggcggcaatatcttctacttcttcccgcaggcagatggttatgttccgccgccggaaaccttagccgcaagtctgatgcgttggaccttcgacctgaatagcgcactggatgaagtggaaccgcagccgctgaccgattatccgtgcgaattcccgcgctgtgatgatcgttatattggtcgtaaatatcagcatggcttcctgctggcattcgatccggaacgtccgtataatccggcaaatggcccgattccgttccagttcttcaatctgctggtgcatctgaatctgaaaaccggcattaccgatgcctggttcccgggcgatagtggctgcttccaggaaccgatcttcattccgcgtagcgcagatgcagaagaagcagatggttacgtggttgccctgctgaatctgattgcagaaggtcgttcagaactggtggttctggatagtcgcgatatggcaagtggtccgattgcacgcattcgcattccgttccgcatgcgcatgagtctgcatggctgttggaccgccgatccgaattaataa

SEQ ID NO: 130 - protein, 483 aa, Pseudomonas viciae
MTIPFPQTPEFSGALYTPSRVEAEVFDLEIEGVLPASIGGTFYQVAPDPQYPPMLGNDIFFNGDGVVSRFNFANGKVSMRRRYVKTDRLLAQRREGRSLNGVYRNVYTNDRLAAKNNTTANTTVIPHNGVLLALKEDALPWAMDLDTLETLGEWTFDGQIKSATFTAHPKLDPVTGNLLACSYEAKGDGTPDLAYFEISPDGKLLHEIWFQAPYAAMVHDFAVTERYVVFPLIPLTVDVERMKNGGPHFQWQPDLPQLFAVVPRNGNAQDVRWFKGPKDGFQGHTLNAFDEDGKVYVDMPVTGGNIFYFFPQADGYVPPPETLAASLMRWTFDLNSALDEVEPQPLTDYPCEFPRCDDRYIGRKYQHGFLLAFDPERPYNPANGPIPFQFFNLLVHLNLKTGITDAWFPGDSGCFQEPIFIPRSADAEEADGYVVALLNLIAEGRSELVVLDSRDMASGPIARIRIPFRMRMSLHGCWTADPN

SEQ ID NO: 131 - DNA, 1452 bp, synthetic construct
  source 1..1452: mol_type = other DNA
atgagcattccgttcccgcagaccccggaattcagtggtgtgctgtataccccgagccgtgtggaagccgaagtgttcgatctggaaattgaaggcattctgccggcaagcatctgtggtaccttctatcaggtggccccggaccctcagtatccgccgatgctgggcaatgatatcttcttcaatggcgatggtgtggtgagtcgcttcaacttcgcaaatggtaaagtgagtatgcgccgccgttatgtgaaaaccgatcgcctgctggcccagcgtcgtgaaggtcgtagtctgaatggtgtgtatcgtaatgtgtataccaatgatccgctggccgccaaaaataataccaccgcaaataccaccgtgattccgcataatggtgtgctgctggcactgaaagaagatgcactgccgtgggcactggatctggaaaccttagaaaccttaggtgaatggaccttcgatggtcagattaaagcagccaccttcaccgcccatccgaaactggaccctgtgaccggtaatctgctggcctgtagttatgaagccaaaggtgatggcaccccggatctggcatacttcgaaattagcccggatggcaaactgctgcatgaaatctggttccaggccccgtatgcagccatggtgcatgacttcgccgttaccgaacgttatgtggtgttcccgctgattccgctgaccgtggatgtggaacgcatgaaaaatggtggtccgcacttccagtggcagccggatctgccgcagctgttcgccattgttccgcgcaatggctgcggtcaggatgttcgctggttcaaaggtccgaaagatggcttccagggccataccctgaatgcattcgatgaagatggtaaagtgtatgtggatatgccggtgaccggtggtaatatcttctacttcttcccgcaggcagatggctatgttccgccgccggaaaccttagccgcaagtctgatgcgttggaccttcgacctgaatggcgcacaggaagatgttcagccgcagccgctgaccgaatatccgtgcgaattcccgcgttgcgatgatcgttatattggccgtaaatatcagcatggcttcctgctggcattcgatccggaacgcccgtataatccggcaaatggcccgattccgttccagttcttcaatctgctggcacatctggatctgcagaccggtcgtaccgatgcctggttcccgggtgatagtggctgcttccaggaaccgatcttcattccgcgtagcgcagatgcagaagaagccgatggttatgttgtggccctgctgaatctgattgcagaaggccgtagtgaactggtggtgctggatacccgtgatatggccagtggcccgattgcccgtattcgcattccgttccgtatgcgtatgagcctgcatggctgctgggcaccgaatgattaataa

SEQ ID NO: 132 - protein, 482 aa, Pseudomonas ogarae
MSIPFPQTPEFSGVLYTPSRVEAEVFDLEIEGILPASICGTFYQVAPDPQYPPMLGNDIFFNGDGVVSRFNFANGKVSMRRRYVKTDRLLAQRREGRSLNGVYRNVYTNDPLAAKNNTTANTTVIPHNGVLLALKEDALPWALDLETLETLGEWTFDGQIKAATFTAHPKLDPVTGNLLACSYEAKGDGTPDLAYFEISPDGKLLHEIWFQAPYAAMVHDFAVTERYVVFPLIPLTVDVERMKNGGPHFQWQPDLPQLFAIVPRNGCGQDVRWFKGPKDGFQGHTLNAFDEDGKVYVDMPVTGGNIFYFFPQADGYVPPPETLAASLMRWTFDLNGAQEDVQPQPLTEYPCEFPRCDDRYIGRKYQHGFLLAFDPERPYNPANGPIPFQFFNLLAHLDLQTGRTDAWFPGDSGCFQEPIFIPRSADAEEADGYVVALLNLIAEGRSELVVLDTRDMASGPIARIRIPFRMRMSLHGCWAPND

SEQ ID NO: 133 - DNA, 1485 bp, Novosphingobium aromaticivorans
  source 1..1485: mol_type = unassigned DNA
atggcccaatttccgaacacccccagcttcacgggattcaacacgccgtcgcggatcgaggcggatatcgccgatctggcccacgaaggcacgattccgcaagggttaaacggcgcattctaccgcgtccagcccgacccgcagtttcctccccgcctcgacgacgacatcgccttcaacggcgacggcatgatcacccgcttccacatccacgacggccaggtcgacttccgccagcgctgggcgaagaccgacaagtggaagctggagaacgccgccggaaaggccctgttcggcgcctaccgcaacccgctgaccgacgacgaggcggtcaagggcgagatccgttcgaccgccaacaccaacgccttcgtgttcggcggcaagctgtgggcgatgaaggaggacagtcccgccctcgtcatggacccggcgacgatggaaaccttcgggttcgagaagttcggcggcaagatgaccggccagacctttaccgcccaccccaaggtcgatccgaagaccggcaacatggtcgccatcggctatgccgcaagcgggctgtgcaccgacgatgtgacctacatggaagtgagcccggagggcgagcttgtccgcgaagtgtggttcaaggtgccgtactactgcatgatgcacgacttcggcatcaccgaggattacctcgtgctgcacatcgtgccttccatcggaagctgggaaaggctggaacagggcaagccgcacttcggcttcgacacgaccatgccggtgcacctcggcatcatcccgcgccgcgacggcgtgcgccaggaagacatccgctggttcacgcgggacaactgctttgccagccatgtcctgaacgcctggcaagaggggaccaagatccacttcgtgacctgcgaggcgaagaacaacatgttcccgttcttccccgacgtccacggcgcgcccttcaacggcatggaggccatgagccatccgaccgactgggtggtcgacatggccagcaacggcgaggactttgccgggatcgtgaagctttccgacacagccgccgagttcccgcgcatcgacgaccgctttaccggccagaagacccgccatggctggttcctcgaaatggacatgaagcgcccggtggaattgcgcggcggcagcgccggcggcctgctgatgaactgcctgttccacaaggacttcgaaacgggtcgcgagcagcactggtggtgcggcccggtgtcgagccttcaggagccgtgcttcgtgccgcgcgccaaggatgcccccgaaggcgacggctggatcgtgcaggtttgcaaccggctggaagagcagcgcagcgacttgctgatcttcgacgcgctcgacatcgagaaaggcccggtggccacggtcaacatccccatccgcctgcgcttcggccttcacggcaactgggcgaatgccgacgaaatcggccttgccgagaaggtcctggccgcatga

SEQ ID NO: 134 - protein, 494 aa, Novosphingobium aromaticivorans
MAQFPNTPSFTGFNTPSRIEADIADLAHEGTIPQGLNGAFYRVQPDPQFPPRLDDDIAFNGDGMITRFHIHDGQVDFRQRWAKTDKWKLENAAGKALFGAYRNPLTDDEAVKGEIRSTANTNAFVFGGKLWAMKEDSPALVMDPATMETFGFEKFGGKMTGQTFTAHPKVDPKTGNMVAIGYAASGLCTDDVTYMEVSPEGELVREVWFKVPYYCMMHDFGITEDYLVLHIVPSIGSWERLEQGKPHFGFDTTMPVHLGIIPRRDGVRQEDIRWFTRDNCFASHVLNAWQEGTKIHFVTCEAKNNMFPFFPDVHGAPFNGMEAMSHPTDWVVDMASNGEDFAGIVKLSDTAAEFPRIDDRFTGQKTRHGWFLEMDMKRPVELRGGSAGGLLMNCLFHKDFETGREQHWWCGPVSSLQEPCFVPRAKDAPEGDGWIVQVCNRLEEQRSDLLIFDALDIEKGPVATVNIPIRLRFGLHGNWANADEIGLAEKVLAA

SEQ ID NO: 135 - protein, 494 aa, synthetic construct
MAQFPNTPSFTGFNTPSRIEADIADLAHEGTIPQGLNGAFYRVQPDPQFPPRLDDDIAFNGDGMITRFHIHDGQVDFRQRWAKTDKWKLENAAGKALFGAYRNPLTDDEAVKGEIRSTANTNAFVFGGKLWAMKEDSPALVMDPATMETFGFEKFGGKMTGQTFTAHPKVDPKTGNMVAIGYAASGLCTDDVTYMEVSPEGELVREVWFKVPYYCMMHDFGITEDYLVLHIVPSIGSWERLEQGKPHFGFDTTMPVHLGIIPRRDGVRQEDIRWFTRDNCFAFHVLNAWQEGTKIHFVTCEAKNNMFPFFPDVHGAPFNGMEAMSHPTDWVVDMASNGEDFAGIVKLSDTAAEFPRIDDRFTGQKTRHGWFLEMDMKRPVELRGGSAGGLLMNCLFHKDFETGREQHWWCGPVSSLQEPCFVPRAKDAPEGDGWIVQVCNRLEEQRSDLLIFDALDIEKGPVATVNIPIRLRFGLHGNWANADEIGLAEKVLAA